(19) United States
(12) Patent Application Publication
(10) Pub. No.: US 2026/0097183 A1
(43) Pub. Date: Apr. 9, 2026

(54) NEUROCATHETERS, SYSTEMS, AND METHODS OF USE

(71) Applicant: Route 92 Medical, Inc., San Mateo, CA (US)

(72) Inventors: Paul De Lipski, San Mateo, CA (US); John Miller, San Mateo, CA (US); Tony M. Chou, San Mateo, CA (US); Stephanie Sharpe, San Mateo, CA (US); Christina Van, San Mateo, CA (US)

(21) Appl. No.: 19/349,736

(22) Filed: Oct. 3, 2025

Related U.S. Application Data
(60) Provisional application No. 63/703,784, filed on Oct. 4, 2024.

(51) Int. Cl. A61M 25/00

(52) U.S. Cl. CPC A61M 25/0053; A61M 25/0045; A61M 25/0051; A61M 2025/0059

(57) ABSTRACT

A catheter comprising a catheter body defining an inner lumen having a working length between a proximal end and a distal end of the catheter body, wherein the catheter body comprises a proximal portion and a distal portion, wherein the proximal portion is stiffer than the distal portion by at least 10×, wherein the catheter is configured to be advanced through a patient's vasculature without a guide sheath so the distal portion reaches an intracranial vessel without prolapse of the proximal portion without aid of a guide sheath. Related devices, systems, kits, and methods are provided.

### RELATED APPLICATIONS

This application claims the benefit of priority under 35 U.S.C. § 119(e) to U.S. Provisional Patent Application Ser. No. 63/703,784, filed Oct. 4, 2024. The disclosure of the patent application is incorporated by reference herein in its entirety.

### FIELD

The present technology relates generally to medical devices and methods, and more particularly, to neurocatheters, systems, and their methods of use.

### BACKGROUND

Acute ischemic stroke (AIS) usually occurs when an artery to the brain is occluded, preventing delivery of fresh oxygenated blood from the heart and lungs to the brain. These occlusions are typically caused by a thrombus or an embolus lodging in the artery and blocking the artery that feeds a territory of brain tissue. If an artery is blocked, ischemia injury follows, and brain cells may stop working. Furthermore, if the artery remains blocked for more than a few minutes, the brain cells may die, leading to permanent neurological deficit or death. Therefore, immediate treatment is critical.

Two principal therapies are employed for treating ischemic stroke: thrombolytic therapy and endovascular treatment. The most common treatment used to reestablish flow or re-perfuse the stroke territory is the use of intravenous (IV) thrombolytic therapy. The timeframe to enact thrombolytic therapy is within 3 hours of symptom onset for IV infusion (4.5 hours in selected patients) or within 6 hours for site-directed intra-arterial infusion. Instituting therapy at later times has no proven benefit and may expose the patient to greater risk of bleeding due to the thrombolytic effect. Endovascular treatment most commonly uses a set of tools to mechanically remove the embolus, without the use of thrombolytic therapy.

The gamut of endovascular treatments include mechanical embolectomy, which utilizes a retrievable structure, e.g., a coil-tipped retrievable stent (also known as a “stent retriever” or a STENTRIEVER), a woven wire stent, or a laser cut stent with struts that can be opened within a clot in the cerebral anatomy to engage the clot with the stent struts, create a channel in the emboli to restore a certain amount of blood flow, and to subsequently retrieve the retrievable structure by pulling it out of the anatomy, along with aspiration techniques. Other endovascular techniques to mechanically remove AIS-associated embolus include Manual Aspiration Thrombectomy (MAT) (also known as the “ADAPT” technique). ADAPT/MAT is an endovascular procedure where large bore catheters are inserted through the transfemoral artery and maneuvered through complex anatomy to the level of the embolus, which may be in the extracranial carotids, vertebral arteries, or intracranial arteries. Aspiration techniques may be used to remove the embolus through the large bore catheters. Another endovascular procedure is Stentriever-Mediated Manual Aspiration Thrombectomy (SMAT) (similar to the Stentriever-assisted “Solumbra” technique). SMAT, like MAT, involves accessing the embolus through the transfemoral artery. After access is achieved, however, a retrievable structure is utilized to pull the embolus back into a large bore catheter.

To access the cerebral anatomy, guide catheters or guide sheaths are used to guide interventional devices to the target anatomy from an arterial access site, typically the femoral artery. The length of the guide is determined by the distance between the access site and the desired location of the guide distal tip. Interventional devices such as guidewires, microcatheters, and intermediate catheters used for sub-selective guides and aspiration, are inserted through the guide and advanced to the target site. Often, devices are used in a co-axial fashion, namely, a guidewire inside a microcatheter inside an intermediate catheter is advanced as an assembly to the target site in a stepwise fashion with the inner, most atraumatic elements, advancing distally first and providing support for advancement of the outer elements. The length of each element of the coaxial assemblage takes into account the length of the guide, the length of proximal connectors on the catheters, and the length needed to extend from the distal end.

Typical tri-axial systems such as for aspiration or delivery of stent retrievers and other interventional devices require overlapped series of catheters, each with their own rotating hemostatic valves (RHV) on the proximal end. For example, a guidewire can be inserted through a Penumbra Velocity microcatheter having a first proximal RHV, which can be inserted through a Penumbra ACE68 having a second proximal RHV, which can be inserted through a Penumbra NeuronMAX 088 access catheter having a third proximal RHV positioned in the high carotid via a femoral introducer. Maintaining the coaxial relationships between these catheters can be technically challenging. The three RHVs must be constantly adjusted with two hands or, more commonly, four hands (i.e., two operators). Further, the working area of typical tri-axial systems for aspiration and/or intracranial device delivery can require working area of 3-5 feet at the base of the operating table. Additionally, it has been shown that the larger the catheter delivered to the clot, the better and faster the aspiration of the clot by that catheter. One of the measures of how quickly and easily the clot is removed is first pass effect (FPE). Catheters with larger inner diameters have shown higher FPE than smaller catheters. The need for multiple coaxial nested catheters to reach the clot results in a larger vessel puncture at the access site than desired and a smaller catheter lumen for clot removal. Therefore, it would be desirable to remove a clot with a system having the minimum outer diameter to minimize access site puncture size while having a maximum inner diameter to maximize clot retrieval.

The time required to access the site of the occlusion and restore, even partially, flow to the vessel is crucial in determining a successful outcome of such procedures. Similarly, the occurrence of distal emboli during the procedure and the potentially negative neurologic effect and procedural complications such as perforation and intracerebral hemorrhage are limits to success of the procedure. There is a need for a system of devices and methods that allow for simplified neurointerventional procedures providing rapid access, optimized catheter aspiration, and treatment to fully restore flow to the blocked cerebral vessel.

### SUMMARY

In an implementation, provided is a catheter having a catheter body defining an inner lumen having a working length between a proximal end and a distal end of the catheter body. The catheter body includes a proximal portion and a distal portion. The proximal portion is stiffer than the distal portion by at least 10×. The catheter is configured to be advanced through a patient's vasculature without a guide sheath so the distal portion reaches an intracranial vessel without prolapse of the proximal portion.

The catheter body can include a multi-layer tube having an inner liner layer, an outer jacket layer, and a reinforcement layer positioned between the inner liner layer and the outer jacket layer that varies in configuration between the proximal portion and the distal portion of the catheter body. The reinforcement layer of the proximal portion includes a hypotube. The reinforcement layer of the distal portion includes a coil reinforcement. The reinforcement layer of the proximal portion can be coupled to the reinforcement layer of the distal portion. The reinforcement layer of the proximal portion is sufficient to prevent prolapse of the catheter body into the descending aorta during advancement of the distal portion into an intracranial vessel. The reinforcement layer of the proximal portion can allow advancement of the distal portion of the catheter body through an intracranial vessel without the use of a long guide catheter.

The hypotube can include a proximal end region and a distal end region. At least the distal end region can have a plurality of cuts. The plurality of cuts in the distal end region can impart a greater flexibility in the distal end region compared to the proximal end region. The plurality of cuts can be perpendicular, substantially perpendicular, or angled relative to a longitudinal axis of the hypotube. The plurality of cuts can create a pattern and the pattern can change periodically continuously over a length of the hypotube to impart a greater flexibility distally compared to proximally. The plurality of cuts can create a plurality of tube segments, the plurality of tube segments having a pitch, pattern, and shape. The pitch, pattern, and/or shape of the plurality of tube segments can be constant along a length of each tube segment of the plurality of tube segments. The pitch, pattern, and/or shape of the plurality of tube segments can vary along a length of each tube segment of the plurality of tube segments. The pitch can increase distally.

The reinforcement layer of the distal portion can include a coil reinforcement. A pitch of the coil reinforcement can vary along a length of the distal portion. The pitch can be narrower proximally and wider distally for increased flexibility toward the distal end of the catheter body. The hypotube can include one or more connecting spirals formed in a distal end of the hypotube. The connecting spirals of the distal end of the hypotube can intersperse with coils of the coil reinforcement of the distal portion to couple the hypotube of the proximal portion to the coil reinforcement of the distal portion.

A length of the proximal portion can be sufficient to position the distal end region of the hypotube to a location distal to the brachiocephalic take-off and proximal of the carotid siphon of the internal carotid artery when the catheter is advanced from a femoral access site. A length of the proximal portion having the hypotube can be sized to avoid advancing substantially into the skull when the catheter is advanced from a femoral access site. A length of the proximal portion having the hypotube can be sized to avoid advancing substantially into the skull when the catheter is advanced from a radial access site. A length of the proximal portion can be sufficient to position the distal end region of the hypotube to a location within about 20 cm to about 30 cm away from an occlusion located in an intracranial vessel. The proximal portion can have a length that is about 70 cm to about 110 cm, preferably about 80 cm to about 100 cm. The distal portion can have a length that is shorter than the length of the proximal portion. The distal portion can have a length that is about 20 cm to about 50 cm. The outer jacket layer in the proximal portion can have a higher durometer segment than a durometer of a segment of the outer jacket layer in the distal portion. The higher durometer segment of the outer jacket layer in the proximal portion can be greater than about 55D. The durometer of the segment of the outer jacket layer in the distal portion can be less than about 25D. The outer jacket layer can include a plurality of segments that transition in durometer between a durometer greater than about 55D and a durometer less than or equal to about 25D. The plurality of segments can include at least 5 segments.

An inner diameter of the inner lumen of the catheter body can be about 0.054″ to about 0.108″, preferably about 0.088″-0.090″. An outer diameter of the catheter body can be about 0.065″ to about 0.110″. The catheter can be configured to be advanced through the patient's vasculature without aid of a guide sheath.

In an interrelated aspect, described is a system including neurocatheter having a catheter body defining an inner lumen and having a working length between a proximal end and a distal end of the catheter body. The catheter body includes a proximal portion reinforced with a cut hypotube and a distal portion reinforced without a hypotube. The proximal portion is stiffer than the distal portion. The system further includes a delivery catheter having a tubular body defining an inner lumen and having a working length between a proximal end and a distal end of the tubular body. The tubular body includes a proximal portion and a tapered distal portion. The delivery catheter is configured to be received within the inner lumen of the catheter body of the neurocatheter for advancing the neurocatheter together with the delivery catheter through at least a portion of the patient's vasculature to reach the middle cerebral artery without aid of a guide sheath.

In an interrelated aspect, described is a system including a neurocatheter having a catheter body defining an inner lumen and having a working length between a proximal end and a distal end of the catheter body. The catheter body includes a proximal portion and a distal portion. The proximal portion is stiffer than the distal portion. The system further includes a navigation catheter having a tubular body defining an inner lumen and having a working length between a proximal end and a distal end of the tubular body. The tubular body includes a proximal portion and a distal portion. The proximal portion is stiffer than the distal portion. The navigation catheter is configured to be received within the inner lumen of the catheter body of the neurocatheter for advancing the neurocatheter together with the navigation catheter through at least a portion of the patient's vasculature without aid of a guide sheath.

In an interrelated aspect, described is a method of accessing an intracranial vessel. The method includes assembling a navigation catheter with a neurocatheter to form a first assembled system of catheters; advancing the first assembled system of catheters through at least a portion of a patient's vasculature from an arterial access site to a first location without aid of a guide sheath, the first location being distal to a brachiocephalic take-off from the aortic arch; withdrawing the navigation catheter from the neurocatheter; advancing a delivery catheter through the neurocatheter; positioning a distal end region of the delivery catheter distal to a distal end of the neurocatheter to form a second assembled system of catheters; and advancing the second assembled system of catheters to at least a second location distal to the first location.

In an interrelated aspect, described is a navigation catheter having a tubular body defining an inner lumen and having a working length between a proximal end and a distal end of the tubular body. The tubular body includes a proximal portion and a distal portion. The proximal portion is stiffer than the distal portion.

The navigation catheter can be configured to be advanced through at least a portion of the patient's vasculature together with a neurocatheter. The tubular body of the navigation catheter minimizes a ledge effect of the neurocatheter.

The tubular body of the navigation catheter can provide steering and vessel selection. The tubular body can include a multi-layer tube having an inner liner layer, an outer jacket layer, and a reinforcement layer positioned between the inner liner layer and the outer jacket layer. The reinforcement layer of the proximal portion can include a braid configured for pushability and torquability of the navigation catheter. A length of the proximal portion having the braid can be sufficient to position the distal end region of the braid to a location distal to the brachiocephalic take-off and proximal of the carotid siphon of the internal carotid artery when the navigation catheter is advanced from a femoral access site. A length of the proximal portion having the braid can be about 120 cm to about 160 cm. A length of the proximal portion having the braid is about 140 cm to about 155 cm. The reinforcement layer of the proximal portion can terminate about 2 cm away from a distal-most end of the tubular body. The inner liner layer can extend through both the proximal portion and the distal portion to a distal opening from the inner lumen. The outer jacket layer in the proximal portion can have a higher durometer than the outer jacket layer durometer in the distal portion. The higher durometer of the outer jacket layer in the proximal portion can be about 72D down to no less than about 55A. The durometer of the outer jacket layer in the distal portion can be about 55A down to no less than about 45D. The tubular body can include a vessel selection tip shape. The tubular body can be shaped and steerable for vessel selection through the aortic arch. An inner diameter of the inner lumen of the tubular body can be sized to receive a guide wire having an outer diameter no greater than about 0.037″. An inner diameter of the inner lumen of the tubular body can be about 0.040″. An outer diameter of the tubular body can be about 0.042″ to about 0.097″. An outer diameter of the tubular body can be about 0.005″ to about 0.010″ smaller than an inner diameter of a neurocatheter it is used with.

In an interrelated aspect, described is a delivery catheter having a tubular body defining an inner lumen and having a working length between a proximal end and a distal end of the tubular body. The tubular body includes a proximal portion and an unreinforced distal portion. The delivery catheter can be configured to be advanced through at least a portion of the patient's vasculature together with a neurocatheters described herein, wherein the tubular body of the delivery catheter minimizes a ledge effect of the neurocatheter.

The proximal portion of the tubular body can include a multi-layer tube having an inner liner layer, an outer jacket layer, and a reinforcement layer positioned between the inner liner layer and the outer jacket layer. The reinforcement layer of the proximal portion can include a braid configured for pushability of the delivery catheter. A length of the proximal portion having the braid can be sufficient to position the distal end region of the braid to a location distal to the brachiocephalic take-off and proximal of the carotid siphon of the internal carotid artery when the delivery catheter is advanced from a femoral access site. A length of the proximal portion having the braid can be about 110 cm to about 130 cm. The reinforcement layer of the proximal portion can terminate about 20 cm-40 cm away from a distal-most end of the tubular body. The inner liner layer can extend through only the proximal portion. The outer jacket layer in the proximal portion can have a durometer of no less than about 72D. A durometer of the distal portion can be no greater than about 35D at a distal-most end of the tubular body. The tubular body can include a tapered tip shape. The tapered tip shape can taper over a length of about 0.5 cm to about 4.0 cm. An inner diameter of the inner lumen of the tubular body can be sized to receive a guide wire having an outer diameter no greater than about 0.018″. An inner diameter of the inner lumen of the tubular body can be about 0.019″ to about 0.021″. An outer diameter of the tubular body can be about 0.042″ to about 0.097″. An outer diameter of the tubular body can be about 0.005″ to about 0.010″ smaller than an inner diameter of the neurocatheters described herein. The delivery catheter can be configured to be advanced so that the distal portion reaches an intracranial vessel when advanced from a femoral access site. The proximal portion of the delivery catheter can be positionable distal to the aortic arch and proximal to a carotid siphon of the internal carotid artery when advanced from a femoral access site. The delivery catheter can be exchanged for the navigation catheters described herein for advancing the neurocatheters described herein through the patient's vasculature without aid of a guide sheath.

In an interrelated aspect, described is a system including a neurocatheter having a catheter body defining an inner lumen and having a working length between a proximal end and a distal end of the catheter body. The catheter body includes a proximal portion and a distal portion. The proximal portion is stiffer than the distal portion. The system further includes a navigation catheter having a tubular body defining an inner lumen and having a working length between a proximal end and a distal end of the tubular body. The tubular body includes a proximal portion and a distal portion. The proximal portion is stiffer than the distal portion. The system further includes a delivery catheter having a tubular body defining an inner lumen and having a working length between a proximal end and a distal end of the tubular body. The tubular body of the delivery catheter has a proximal portion and an unreinforced distal portion. The navigation catheter is configured to be received within the inner lumen of the catheter body of the neurocatheter for advancing the neurocatheter together with the navigation catheter through at least a portion of the patient's vasculature without aid of a guide sheath to a first location. The delivery catheter is configured to be received within the inner lumen of the catheter body of the neurocatheter in exchange for the navigation catheter for advancing the neurocatheter together with the delivery catheter through at least a portion of the patient's vasculature without aid of a guide sheath to at least a second location that is further distal than the first location.

The navigation catheter can be sized to be positionable within the inner lumen of catheter so that at least a segment of the distal portion of the navigation catheter extends distal to a distal-most end of the catheter for insertion through a peripheral artery via an arterial access site to the first location. The arterial access site can be a femoral artery access site. The delivery catheter can be sized to be positionable within the inner lumen of catheter so that at least a segment of the distal portion of the delivery catheter extends distal to a distal-most end of the catheter for advancement of the catheter to the second location. The first location can be distal to the brachiocephalic take-off and proximal of the carotid siphon. The second location can be intracranial segment of the internal carotid artery. A distal end of the distal portion of the navigation catheter can be more rigid than a distal end of the distal portion of the neurocatheter and a distal end of the distal portion of the navigation catheter can be more flexible than a distal end of the distal portion of the neurocatheter. The distal end of the distal portion of the navigation catheter can be at least two times or at least four times more rigid than a distal end of the distal portion of the delivery catheter. The combination of the distal portions of navigation catheter and the neurocatheter can be at least two times or at least four times more rigid than the combination of the distal portions of the neurocatheter and the delivery catheter.

A difference between an inner diameter of the neurovascular catheter and an outer diameter of the navigation catheter can be about 0.006″ or less. A difference between an inner diameter of the neurovascular catheter and an outer diameter of the delivery catheter can be about 0.006″ or less. The tubular body of the navigation catheter can have an angled tip which provides steering and vessel selection and wherein the tubular body of the delivery catheter can have a straight tapered flexible tip.

In an interrelated implementation, described is an intravascular catheter having a catheter body having a proximal section, a distal section, and a transition section disposed between the proximal section and the distal section. The proximal section of the catheter body includes a proximal end region of a metallic hypotube having a first plurality of cuts forming a first plurality of tube segments. The proximal end region having a proximal bending stiffness along a proximal length that is greater than about 10 N. The transition section of the catheter body includes a distal end region of the metallic hypotube having a second plurality of cuts forming a second plurality of tube segments. The distal end region has a distal bending stiffness and a distal length. The distal bending stiffness changes over the distal length by at least 10×, the distal length being less than about 30 cm. The distal section of the catheter body has a flexible polymer tube including a coil reinforcement disposed between an inner polymer layer and an outer polymer layer.

The catheter body can have a working length of at least 110 cm to about 135 cm and wherein the metallic hypotube has a length of about 90 cm to about 115 cm. The proximal length can be up to about 80 cm. The distal section can have a bending stiffness in a range that allows extension of the distal section of the catheter body through an intracranial vessel. The proximal section can have a bending stiffness range that prevents prolapse of the proximal section of the catheter body into an aorta upon advancement of the distal section into the intracranial vessel.

In an interrelated aspect, provided is a neurocatheter having a catheter body defining an inner lumen having a working length between a proximal end and a distal end of the catheter body. The catheter body includes a proximal section, a transition section and a distal section. The proximal section is stiffer than the distal section by at least 10×. The catheter is capable of being advanced through a patient's vasculature to the neurovasculature without being positioned inside an outer catheter except for a short introducer sheath at the access location having a length less than about 30 cm so the distal section reaches the middle cerebral artery without prolapse of the proximal section.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes, it should be readily understood that such features are not intended to be limiting. The claims that following the disclosure are intended to define the scope of the protected subject matter.

### BRIEF DESCRIPTION OF THE DRAWINGS

These and other aspects will now be described in detail with reference to the following drawings. Generally speaking the figures are not to scale in absolute terms or comparatively, but are intended to be illustrative. Also, relative placement of features and elements may be modified for the purpose of illustrative clarity.

FIG. 1A illustrates the course of the terminal internal carotid artery through to the cerebral vasculature;

FIG. 1B illustrates the aortic arch including the take-offs of the brachiocephalic BT, left common carotid LCC, and left subclavian arteries LSA from the aortic arch AA;

FIG. 1C illustrates a catheter system including a large bore neurocatheter and a navigation catheter inserted through a femoral access site and extending to vessel located distal of the aortic arch and proximal of the carotid siphon;

FIG. 1D illustrates a catheter system including a large bore neurocatheter and a delivery catheter advanced to an intracranial vessel distal of the carotid siphon;

FIG. 2 is a side view of an implementation of a large bore neurocatheter, an implementation of a navigation catheter, and an implementation of a delivery catheter, one or more of which providing a system of neurointerventional devices;

FIG. 3A is a side view of the neurocatheter of FIG. 2;

FIG. 3B are longitudinal cross-sectional views taken along line B-B at various locations of the neurocatheter of FIG. 3A;

FIG. 3C is a schematic illustrating a transition between a hypotube and a coil of the neurocatheter of FIG. 3A;

FIG. 3D is a schematic illustrating an implementation of a hypotube for incorporating into a catheter of FIG. 3A;

FIG. 3E is a schematic illustrating another implementation of a hypotube for incorporating into a catheter of FIG. 3A;

FIG. 3F is a schematic illustrating another implementation of a hypotube for incorporating into a catheter of FIG. 3A;

FIG. 3G is a schematic illustrating another implementation of a hypotube for incorporating into a catheter of FIG. 3A;

FIG. 4 is a longitudinal cross-sectional, partial view of the navigation catheter of circle 4-4 of FIG. 2 taken along line 4-4;

FIG. 5A is a longitudinal cross-sectional, partial view of the delivery catheter of FIG. 2 taken along line 5-5;

FIG. 5B is a detailed cross-sectional view of the catheter of FIG. 5A taken at circle B-B;

FIG. 5C is a detailed cross-sectional view of the catheter of FIG. 5A taken at circle C-C;

FIG. 6A is a longitudinal cross-sectional, partial view of an assembled catheter system including the large bore neurocatheter and the navigation catheter of FIG. 2;

FIG. 6B is a longitudinal cross-sectional, partial view of an assembled catheter system including the large bore neurocatheter and the delivery catheter of FIG. 2;

FIGS. 7-8 illustrates the bending flexibility of various catheters as assessed by three-point bend testing.

It should be appreciated that the drawings are for example only and are not meant to be to scale. It is to be understood that devices described herein may include features not necessarily depicted in each figure.

### DETAILED DESCRIPTION

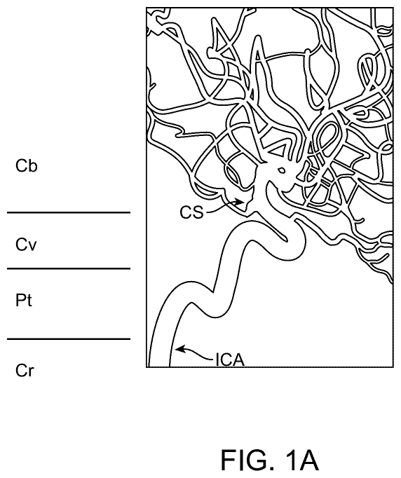

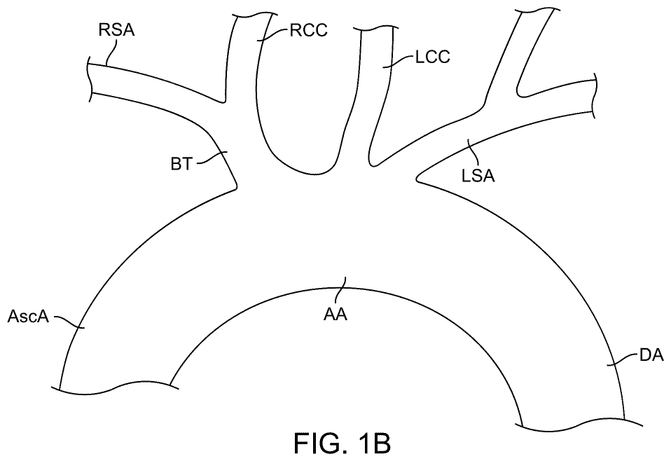

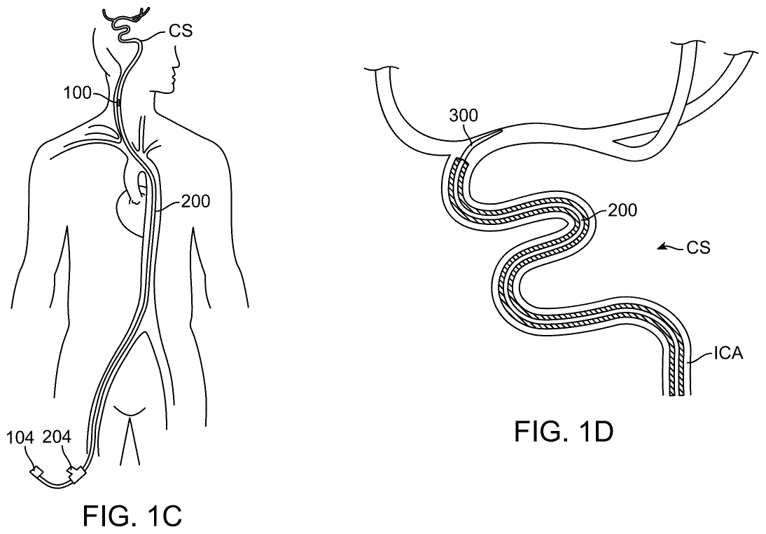

Navigating the carotid anatomy in order to treat various neurovascular pathologies at the level of the cerebral arteries, such as acute ischemic stroke (AIS), requires catheter systems having superior flexibility and deliverability. The internal carotid artery (ICA) arises from the bifurcation of the common carotid artery at the level of the intervertebral disc between C3 and C4 vertebrae. As shown in FIG. 1A, the course of the ICA is divided into four parts-cervical Cr, petrous Pt, cavernous Cv and cerebral Cb parts. In the anterior circulation, the consistent tortuous terminal carotid is locked into its position by bony elements. The cervical carotid Cr enters the petrous bone and is locked into a set of turns as it is encased in bone. The cavernous carotid is an artery that passes through a venous bed, the cavernous sinus, and while flexible, is locked as it exits the cavernous sinus by another bony element, which surrounds and fixes the entry into the cranial cavity. Because of these bony points of fixation, the petrous and cavernous carotid (Pt and Cv) and above are relatively consistent in their tortuosity. The carotid siphon CS is an S-shaped part of the terminal ICA. The carotid siphon CS begins at the posterior bend of the cavernous ICA and ends at the ICA bifurcation into the anterior cerebral artery ACA and middle cerebral artery MCA. The ophthalmic artery arises from the cerebral ICA, which represents a common point of catheter hang-up in accessing the anterior circulation. The MCA is initially defined by a single M1 segment and then further bifurcates in two or three M2 segments and then further arborizes to create M3 segments. These points of catheter hang up can significantly increase the amount of time needed to restore blood perfusion to the brain, which in the treatment of AIS is a disadvantage with severe consequences.

With advancing age, the large vessels often enlarge and lengthen. Fixed proximally and distally, the cervical internal carotid artery often becomes more tortuous with age. The common carotid artery is relatively fixed in the thoracic cavity as it exits into the cervical area by the clavicle. The external and internal carotid arteries ECA, ICA are not fixed relative to the common carotid artery, and thus they develop tortuosity with advancing age with lengthening of the entire carotid system. This can cause them to elongate and develop kinks and tortuosity or, in worst case, a complete loop or so-called “cervical loop”. If catheters used to cross these kinked or curved areas are too stiff or inflexible, these areas can undergo a straightening that can cause the vessel to wrap around or “barbershop pole” causing focused kinking and folding of the vessel. These sorts of extreme tortuosity also can significantly increase the amount of time needed to restore blood perfusion to the brain, particularly in the aging population. In certain circumstances, the twisting of vessels upon themselves or if the untwisted artery is kinked, normal antegrade flow may be reduced to a standstill creating ischemia. Managing the unkinking or unlooping the vessels such as the cervical ICA can also increase the time it takes to perform a procedure.

A major drawback of current catheter systems and methods for stroke intervention procedures is the amount of time required to restore blood perfusion to the brain, including the time it takes to access the occlusive site or sites in the cerebral artery and the time it takes to completely remove the occlusion in the artery. Because it is often the case that more than one attempt must be made to completely remove the occlusion, reducing the number of attempts as well as reducing the time required to exchange devices for additional attempts is an important factor in minimizing the overall time. Additionally, each attempt is associated with potential procedural risk due to device advancement in the delicate cerebral vasculature. Another limitation is the need for multiple operators to deliver and effectively manipulate long tri-axial systems with multiple RHVs typically used with conventional guide and distal access catheters.

Described herein are neurocatheters and catheter systems for treating various neurovascular pathologies, such as acute ischemic stroke (AIS). The catheters and systems described herein provide quick and simple single-operator access to distal target anatomy, in particular tortuous anatomy of the cerebral vasculature. The catheters and systems described herein allow for navigating complex, tortuous anatomy to perform rapid and safe aspiration and removal of cerebral occlusions for the treatment of acute ischemic stroke. The catheters and systems described herein can also be used to deliver intracranial medical devices, with or without aspiration for the removal of cerebral occlusions in the treatment of acute ischemic stroke. The catheters and systems described herein can be particularly useful for the treatment of AIS whether a user intends to perform stent retriever delivery alone, aspiration alone, or a combination of aspiration and stent retriever delivery as a frontline treatment for AIS. Further, the extreme flexibility and deliverability of the neurocatheters and systems described herein allow the catheters to take the shape of the tortuous anatomy rather than exert straightening forces creating new anatomy. The neurocatheters and systems described herein can pass through tortuous loops while maintaining the natural curves of the anatomy therein decreasing the risk of vessel straightening. The neurocatheters and systems described herein can thereby create a safe conduit through the neurovasculature maintaining the natural tortuosity of the anatomy for other catheters to traverse (e.g., interventional device delivery catheters). The catheters and systems that traverse the conduit need not have the same degree of flexibility and deliverability such that if they were delivered directly to the same anatomy rather than through the conduit, would lead to straightening, kinking, or folding of the anterior circulation.

While some implementations are described herein with specific regard to accessing a neurovascular anatomy or delivery of treatment devices, the catheters, systems and methods described herein should not be limited to this and may also be applicable to other uses. For example, the catheters and catheter systems described herein may be used to deliver working devices to a target vessel of a coronary anatomy, peripheral anatomy, or other vasculature anatomy. Coronary vessels are considered herein including left and right coronary arteries, posterior descending artery, right marginal artery, left anterior descending artery, left circumflex artery, M1 and M2 left marginal arteries, and D1 and D2 diagonal branches. Any of a variety of peripheral vessels are considered herein including the popliteal arteries, anterior tibial arteries, dorsalis pedis artery, posterior tibial arteries, and fibular artery.

Where the phrase “aspiration catheter” is used herein, a catheter may be used for other purposes besides or in addition to aspiration, such as the delivery of fluids to a treatment site or as a support catheter or distal access catheter providing a conduit that facilitates and guides the delivery or exchange of other devices such as a guidewire or interventional devices, such as stent retrievers. Alternatively, the catheters and systems described herein may also be useful for access to other parts of the body outside the vasculature.

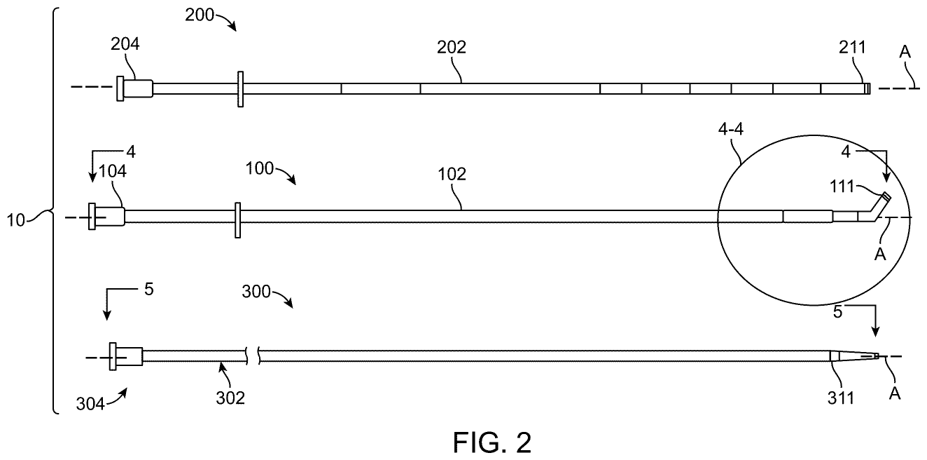

Referring now to the drawings, FIG. 2 illustrates a catheter system 10 including devices for accessing and removing a cerebral occlusion to treat acute ischemic stroke from an access site. The system 10 can include one or more of a large bore catheter 200 for neurovascular use as an interventional neurocatheter and/or a long guide catheter, a navigation catheter 100, and one or more of a delivery catheter 300 corresponding in size to the large bore catheters 200, each of which will be described in more detail below.

Conventional neurocatheter systems for stroke intervention typically include a long guide sheath or long guide catheter placed through a shorter “introducer” sheath (e.g., an 8Fr sheath that is about 11-30 cm in length that passes the vessel wall) at the groin. The long guide sheath is typically positioned in the ICA to support neurovascular interventions including stroke embolectomy (sometimes referred to as “thrombectomy”). For added support, these can be advanced up to the bony terminal petrous and, rarely, into the cavernous or clinoid or supraclinoid terminal ICA when possible. To reach targets in the M1 or M2 distribution for ADAPT/MAT or Solumbra/SMAT approaches, an additional catheter may be inserted through the long guide catheter. These catheters are typically large-bore aspiration catheters that can be, for example, about 130 cm in working length or longer.

As will be described in more detail below, the catheter systems 10 described herein are capable of being advanced through a patient's vasculature without the aid of a long guide sheath. The large bore catheter 200 is designed to have exceptional deliverability and is configured to be advanced directly through the vessels to the level of the M1 or M2 without any guide sheath support. The catheter 200 can have a relatively large inner diameter suitable for drawing higher aspiration forces for direct aspiration thrombectomy and/or delivery of larger interventional devices for treating stroke and other neurovascular conditions more quickly with lower risk. The catheter 200 incorporates a proximal portion reinforced for pushability and support that needs no guide sheath support within the vessel. The catheter 200 also incorporates a distal portion reinforced for maximum flexibility to navigate intracranial vessels without collapse or kinking. The catheter 200 provides a maximum distal inner diameter for improved treatment and smaller proximal outer diameter for reduced arteriotomy size because no guide sheath is necessary to advance the catheter to distal sites. The catheter 200 provides improved fluid dynamics for treating stroke more effectively due to fewer telescoping components, which additionally decreases the time needed to reach distal sites for treatment. These advantages alone provide better outcomes for patients.

The catheter 200 is advanced using one or more exchangeable catheters 100, 300 that also improve delivery and outcomes. The navigation catheter 100 aids delivery of the catheter 200 to reach sites distal to the aortic arch quickly and safely. The delivery catheter 300 allows the catheter 200 to advance through intracranial vessels quickly and safely without catching on branched vessels within the tortuous anatomy of the skull.

Although unnecessary, the system 10 may further include or be used with a separate short or long guide sheath, if desired. The system 10 may further include a short introducer sheath (e.g., an 8Fr sheath that is about 11-30 cm in length that passes the vessel wall or a peel-away sheath), if desired. The catheters described herein are also capable of being advanced without a guidewire, whether pre-positioned or parked within the lumen of the catheter being delivered. Nevertheless, the system 10 may further include or be used with one or more guidewires, if desired.

The catheters 100, 200, 300 described herein are designed to be used individually for a variety of purposes or in a variety of combinations as a catheter system. For example, the system 10 may include the navigation catheter 100 together with the catheter 200 or the delivery catheter 300 together with the catheter 200. The system 10 may include the navigation catheter 100 together with the delivery catheter 300. The system 10 may include the navigation catheter 100, the delivery catheter 300, and the catheter 200. The navigation catheter 100 is capable of being used alone, such that the system 10 may include only the navigation catheter 100. The catheter 200 is capable of being used alone, such that the system 10 may include only the catheter 200. The delivery catheter 300 is capable of being used alone, such that the system 10 may include only the delivery catheter 300. Any of a variety of combinations are considered herein.

Each of the various components will now be described in more detail.

### Large Bore Catheters

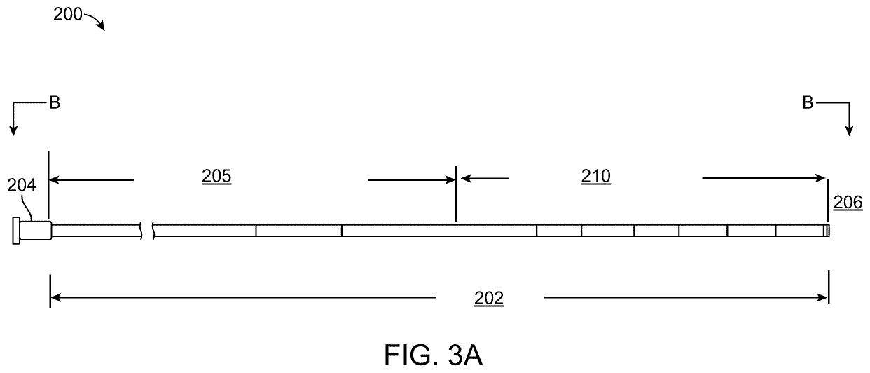

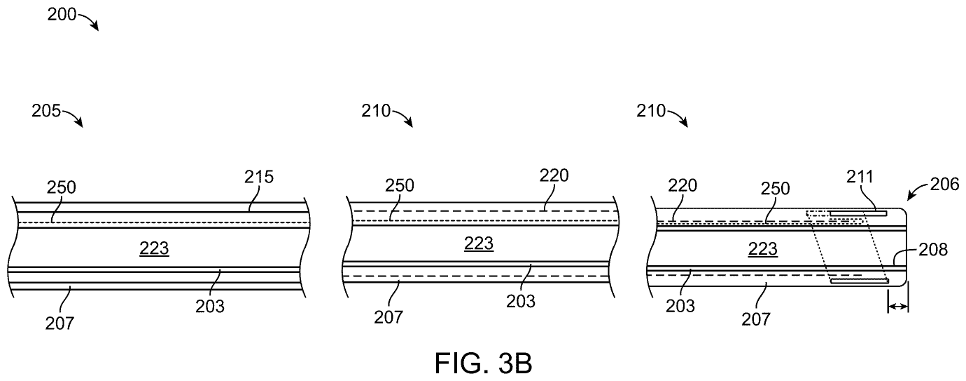

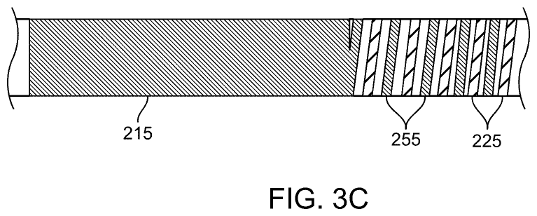

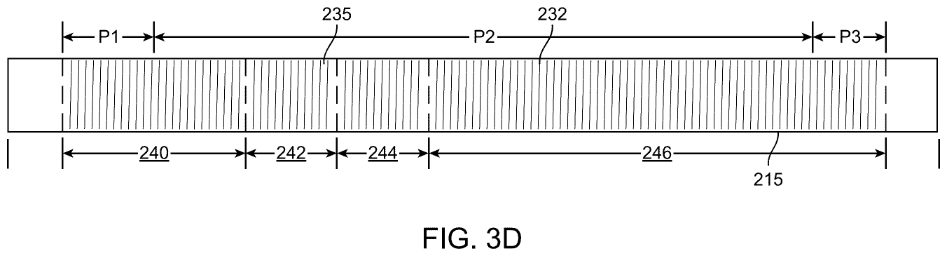

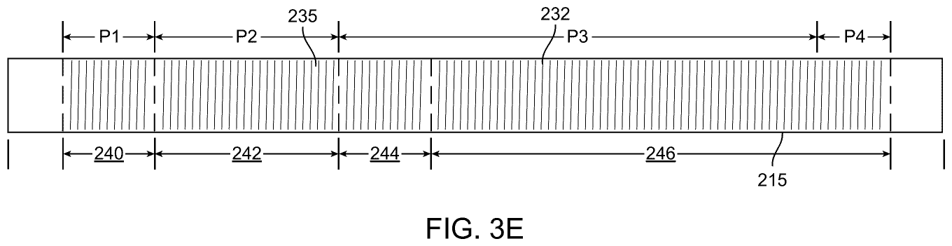

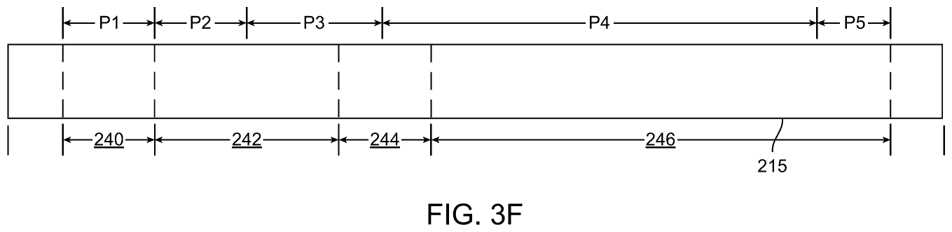

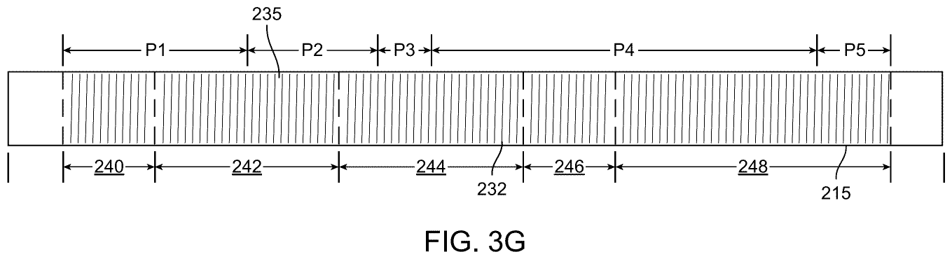

The catheter 200 of the catheter system 10 can be a large bore catheter configured to be used to deliver aspiration and/or other catheters or interventional working devices, such as those configured to provide thrombotic treatments, wires, balloons, retrievable structures such as coil-tipped retrievable stents “Stentriever” as well as permanent structures including flow diverters, and vessel support implants including balloon expandable stents, self-expanding stents, and mesh sleeves. The catheter 200 provides the functions of both a flexible neurointerventional catheter or aspiration catheter and the functions of a guide sheath and can be considered a combined one-piece neurointerventional catheter and guide sheath. The catheter 200 can also be referred to as a sheathless neurointerventional catheter and the system of catheters including one or more of the navigation catheter 100, catheter 200, delivery catheter 300 shown in FIG. 2 can be referred to as a sheathless neurointerventional system because the catheter and systems do not require the use of a traditional long access sheath for proximal support. Again with respect to FIG. 2 and also FIGS. 3A-3C, the catheter 200 can include a catheter body 202 defining an inner lumen 223 having a working length between a proximal end and a distal end of the catheter 200. The catheter body 202 includes a proximal portion 205 that is a relatively stiff shaft that transitions to an increasingly flexible distal portion 210. The catheter body 202 of the catheter 200 is configured to assume and navigate the bends of the vasculature without causing vascular trauma and without kinking or collapsing even when, for example, subjected to high aspiration forces, which will be described in further detail below. The characteristics of the catheter 200 can be selected for virtually any intravascular application, but the catheters 200 are particularly suited for and designed to be used to traverse the cerebral vasculature for aspiration thrombectomy through its large inner diameter without kinking or collapse.

The catheter body 202 can be a multi-layer tube. For example, the catheter body 202 can include a lubricious inner liner layer, an outer jacket layer, and a reinforcement layer that lies between the inner liner layer and the outer jacket layer. One or more of the layers can vary along the length of the catheter to provide different functional capabilities of the catheter 200. For example, the outer jacket layer can incorporate discreet sections of polymer with different durometers, compositions, and/or thicknesses to aid in varying the flexibility along the length of the catheter 200. The reinforcement layer also can vary along the length, such that at least a first reinforcement type is located within the proximal portion 205 of the catheter and at least a second reinforcement type is located within the distal portion 210 of the catheter. FIG. 3B is a schematic illustrating the different layers of the catheter 200 in the proximal portion 205 and in the distal portion 210. FIG. 3C is a schematic illustrating a transition between the reinforcement layer of the proximal portion 205, which can be a metal hypotube 215, such as the cut hypotubes described in more detail below, and the reinforcement layer of the distal portion 210, which can be reinforced without a hypotube, such as a metal coil reinforcement 220. The proximal hypotube 215 can at least a distal end region that is laser cut (cuts not visible in FIG. 3C). The proximal end region of the hypotube 215 can be solid or also incorporate laser cuts. In addition to the laser cut region, the hypotube 215 can incorporate spiral cuts at the distal end creating two, three, four, five, or more coils 255 designed to intersperse with coils 225 of the coil reinforcement 220 the neurocatheter 200 of FIG. 3A, which will be described in more detail below. The inner liner layer 203 separates the hypotube 215 from the inner lumen 223 in the proximal portion 205 and separates the coil reinforcement 220 from the inner lumen 223 in the distal portion 210. The outer jacket layer 207 overlays the hypotube 215 in the proximal portion 205 and overlays the coil reinforcement 220 in the distal portion 210. The reinforcement layer within the proximal portion 205 of the catheter body 202 (e.g., hypotube-reinforced shaft) is designed specifically for pushability and torqueability to intracranial vessels and the reinforcement layer within the distal portion 210 of the catheter body 202 (e.g., coil-reinforced shaft) is designed specifically for exceptional flexibility to navigate turns of the intracranial vessels and to avoid collapse upon drawing high aspiration forces for thrombectomy.

The proximal portion 205 of the catheter body 202 is reinforced for pushability and support so that the system needs no long guide sheath support within the vessel. The stiffness within the proximal portion 205 is sufficient to avoid problems of prolapse of the catheter 200, which can be particularly problematic when advancing a catheter through tortuous anatomy alone through a vessel without a guide sheath present, while at least a portion of the reinforcement layer of the proximal portion 205 is designed to navigate the curves of the aortic arch to advance distal to the brachiocephalic take-off. For example, a catheter may be advanced through curves of a vessel and the distal end region of the catheter be met with resistance. Continued advancement against this resistance creates opposing forces, which can lead to buckling and prolapse points within more proximal regions of the catheter. For example, if the distal end of a catheter meets resistance in the carotid siphon (CS), the proximal end region of the catheter can prolapse down into the ascending aorta (AscA) because no guide sheath is there to support that proximal portion. The stiffness of the proximal portion 205 of the catheter 200 prevents prolapse during navigation of tortuous intracranial vessels. The proximal stiffness (alone or together with the presence of the navigation catheter 100 or delivery catheter 300 to be described in detail below) enhances deliverability of the catheter 200 to intracranial sites without the supportive presence of the guide sheath.

The reinforcement within the proximal portion 205 and the distal portion 210 of the catheter 200 as well as the transition between these portions will be discussed in more detail below.

### Proximal Portion Reinforcement

As mentioned, the proximal portion 205 of the catheter body 202 is reinforced to be relatively stiff for pushability and torqueability compared to the flexible distal portion 210. FIG. 3A is a side view of the catheter 200 showing the proximal portion 205 and the distal portion 210 of the catheter body 202. In an implementation, the reinforcement layer of the proximal portion 205 incorporates a hypotube 215 at least a distal end region being cut or incorporating a plurality of cuts 232 configured to impart an increased flexibility to the distal end region (see FIGS. 3B-3C). The proximal end region of the hypotube 215 is relatively stiff, heat resistant, and resistant to kinking. The proximal end region of the hypotube 215 provides stiffness to the proximal portion 205 of the catheter 200 so that the proximal portion 205 can be used to advance the catheter 200 through a vessel, particularly through tortuous anatomy of the intracranial vessels, without any long guide sheath.

The proximal portion 205 of the catheter 200 reinforced by the hypotube 215 can be long enough to extend from the access location (e.g., transfemoral) to within at least a portion of the internal carotid artery (ICA) while also providing for adjustments, if needed. The hypotube 215 can project from a hub strain relief at the proximal end of the catheter 200 up to a distance that is less than the total working length of the catheter 200 so that the hypotube 215 does not extend to the distal end of the catheter. The length of the hypotube 215 relative to the total working length of the catheter 200 ensures the hypotube 215 avoids advancing into the skull when the distal portion reaches distal sites, such as the M1 or M2 level of the cerebral vessels. In some implementations (e.g., femoral or radial percutaneous access), the reinforced proximal portion 205 can be about 60 cm to about 140 cm, or about 80 cm to about 120 cm, preferably about 90 cm to about 110 cm, most preferably about 90 cm to about 105 cm. In an implementation, the catheter 200 has a working length of about 110 cm to about 135 cm, preferably 115 cm-125 cm, and the total length of the hypotube 215 from the proximal hub that is about 85 cm-120 cm, preferably about 90 cm-115 cm. The length of the hypotube 215 of the proximal portion 205 is sufficient to navigate the curves of the aortic arch and advance distal to the brachiocephalic take-off. The length of the hypotube 215 preferably avoids advancing substantially into the skull and thus, extends up to a location that is just proximal of the carotid siphon. The stiffness of the proximal portion 205 provided by the hypotube 215 is sufficient to prevent prolapse of the catheter 200 back down into the aorta, which can be particularly problematic when advancing a catheter through tortuous anatomy alone through a vessel without a guide sheath present. The stiffness of the hypotube 215 transitions distally to allow navigation around the aortic arch and up into the brachiocephalic take-off.

The hypotube 215 can be a super elastic material such as nickel titanium alloy or other suitable stiff material, such as stainless steel. The hypotube 215 can have an inner diameter of about 0.065″ to about 0.105″, preferably about 0.090″ to about 0.095″, an outer diameter of about 0.070″ to about 0.110″, preferably about 0.100″ to about 0.104″ and a wall thickness of about 0.0015″ to about 0.010″, preferably about 0.003″ to about 0.005″.

The hypotube 215 can incorporate cuts along at least a portion of its length, preferably within the distal end region. In some implementations, a proximal end region of the hypotube 215 is a solid hypotube without interruptions through its sidewall for stiffness and the distal end region incorporates interruptions (e.g., slots, cuts, perforations, etc.) for flexibility. Preferably, both the proximal end region and the distal end region of the hypotube 215 incorporates cuts 232 through the sidewall in one or more locations. The cuts 232 can be designed to provide a transition from the stiffness of the proximal end region of the hypotube 215 to the flexibility of the coil-reinforced distal portion 210 of the catheter. As discussed above, the hypotube 215 can have a length that allows it to make the turns of the aortic arch and the brachiocephalic take-off into the internal carotid artery. To provide flexibility to the region of the hypotube making these turns, the hypotube 215 can incorporate a plurality of cuts 232 along its length (see FIGS. 3D, 3E, and 3G). For a catheter 200 that is about 115 cm-125 cm long, the hypotube 215 can extend about 90 cm-115 cm at least 90%-100% of the hypotube 215 incorporates cuts 232 for flexibility. The hypotube 215 can additionally incorporate spiral cuts at its distal end creating the coils 255 that intersperse and interlock with the coil 225 of the distal portion 210 of the catheter 200.

The cuts 232 can be perpendicular, substantially perpendicular, or angled with respect to the longitudinal axis of the tube. In a preferred implementation, the cuts 232 extend in a non-perpendicular direction relative to the longitudinal axis of the hypotube 215. The cuts 232 can create continuous spiral patterns, interrupted spiral patterns, interlocking spirals, hinged patterns and the like. The cuts 232 can be straight cuts, slots, angled cuts, and intricate patterns. A pattern of the cuts 232 can change over the length of the tube, which is described in more detail below. In an implementation, the pattern of the cuts 232 can change continuously over a length of the tube or over a portion of the length of the tube toward its distal end. For example, the cuts 232 can be closer together for greater flexibility the further distal along the hypotube such that regions more proximally have cuts 232 that are further apart than regions more distally so that the distal end region has a greater flexibility. The cuts 232 can overlap to a greater degree near the distal end of the tube.

In an implementation, the cuts 232 in the distal end region of the hypotube 215 create a plurality of tube segments 235 having a variety of shapes and patterns depending on the shape and pattern of the cuts 232 (see FIGS. 3D, 3E, and 3G). The tube segments 235 formed by the cuts 232 can have a pitch between about 0.001″ and about 0.05″, preferably about 0.003″ and about 0.016″. The segments 235 can be constant in pitch along the length of the distal end region of the hypotube 215. The segments 235 preferably vary in pitch along the length of the distal end region of the hypotube 215. The segments 235 can have 2, 3, 4, or more different pitches along the length of the cut section of hypotube 215. The pitch can increase over the length towards the distal-most end of the hypotube 215.

An example is shown in FIG. 3D. The left-hand side of the figure is more distal and the right-hand side of the figure is more proximal. A first section 240 of the plurality of segments 235 starting near a distal end of the hypotube 215 can have a first pitch, which varies along the first section 240 between about 0.003″-0.0045″, a second section 242 of the plurality of segments 235 moving proximally can have a longitudinally varying second pitch of between about 0.0045″-0.008″, a third section 244 of the plurality of segments 235 moving proximally can have a third pitch varying between about 0.008″-0.015″, and a fourth section 246 of the plurality of segments 235 moving proximally can have a varying or substantially constant fourth pitch of about 0.015″. The spiral cuts forming the coils 255 are not shown in FIG. 3D. The distal-most section without any cuts 232 illustrated distal to the first section 240 could be used to form the coils 255 by the spiral cuts.

As another example shown in FIG. 3E, the first section 240 of the plurality of segments 235 starting near the distal end of the hypotube 215 can have a first pitch, which varies along the first section 240 between about 0.003″-0.005″, the second section 242 of the plurality of segments 235 moving proximally can have a longitudinally varying second pitch of between about 0.005″-0.010″, the third section 244 of the plurality of segments 235 moving proximally can have a third pitch varying between about 0.010″-0.016″, and the fourth section 246 of the plurality of segments 235 moving proximally can have a varying or substantially constant fourth pitch of about 0.016″. The spiral cuts forming the coils 255 are not shown in FIG. 3E. The distal-most section without any cuts 232 illustrated distal to the first section 240 could be used to form the coils 255 by the spiral cuts.

FIG. 3F illustrates yet another example of a pattern. The pitch of the segments 235 in each section can be substantially constant, continuously varying or partially varying and partially constant. A continuously varying pitch pattern provides the smoothest transition in flexibility.

In still further examples shown in FIG. 3G, the first section 240 of the plurality of segments 235 starting near the distal end of the hypotube 215 can have a first pitch, which varies along the first section 240 between about 0.003″-0.0037″, the second section 242 of the plurality of segments 235 moving proximally can have a longitudinally varying second pitch of between about 0.0037″-0.005″, the third section 244 of the plurality of segments 235 moving proximally can have a longitudinally varying second pitch of between about 0.005″-0.010″, the fourth section 246 of the plurality of segments 235 moving proximally can have a third pitch varying between about 0.010″-0.016″, and the fifth section 248 of the plurality of segments 235 moving proximally can have a varying or substantially constant fourth pitch of about 0.016″. The spiral cuts forming the coils 255 are not shown in FIG. 3G. The distal-most section without any cuts 232 illustrated distal to the first section 240 could be used to form the coils 255 by the spiral cuts.

The pattern, shape, cut/uncut pattern of the cuts 232 and the resulting pitch, shape, and pattern of the segments 235, can each be constant over the length or section or can vary along the length or section of the hypotube. For example, the cuts 232 can have at least 1, 2, 3, 4, or more patterns. In addition, the same hypotube can have 1, 2, 3, 4, 5 or more pitches in the same or overlapping sections. As an example, FIG. 3D shows a first pattern P1 of cuts 232 near the distal end of the hypotube 215 (left-hand side of figure) can be about 120-150 degrees cut and about 10 degrees uncut and extends along a first length. A degree of a cut 232 as described herein is relative to a circumference of the hypotube 215 such that 120 degrees is ⅓ of the circumference of the tube, 180 degrees is ½ of the circumference of the tube, 240 degrees is ⅔ of the circumference of the tube, and so on. Moving proximally, a second pattern P2 of cuts 232 can be about 110-140 degrees cut and about 20 degrees uncut and extends along a second length. Moving further proximally, a third pattern P3 of cuts 232 can be varied along a third length and be about 110-140 degrees cut and about 20 degrees uncut to about 90-125 degrees cut and about 30-50 degrees uncut at the most rigid proximal end.

As another example, shown in FIG. 3E, a first pattern P1 of cuts 232 near the distal end of the hypotube 215 can be 135 degrees cut and 10 degrees uncut and extend along a first length. Moving proximally, a second pattern P2 of cuts 232 can be 135 degrees cut and 10 degrees uncut to 120 degrees cut and 25 degrees uncut and extend along a second length. Moving further proximally, a third pattern P3 of cuts 232 can be 120 degrees cut and 25 degrees uncut and extend along a third length. Even further proximally, a fourth pattern P4 of cuts 232 can be 120 degrees cut and 25 degrees to 100 degrees cut and 45 degrees uncut and extend along a fourth length. As a still further example, a first pattern P1 of cuts 232 near the distal end of the hypotube 215 can be 135-140 degrees cut and 5-10 degrees uncut and extend along a first length. Moving proximally, a second pattern P2 of cuts 232 can be 120-135 degrees cut and 10-25 degrees uncut and extend along a second length. Moving further proximally, a third pattern P3 of cuts 232 can be 120 degrees cut and 25 degrees uncut and extend along a third length. Even further proximally, a fourth pattern P4 of cuts 232 can be 100-120 degrees cut and 25-45 degrees uncut and extend along a fourth length.

The lengths the patterns P1-P5 extend can be different from the lengths the sections of cuts 240-248 extend so that transitions from one section to the next section of cuts are off-set from the transitions from one pattern to the next pattern. For example and as shown in FIG. 3D, a catheter that is 115-125 cm long can include a first section 240 of cuts 232 that is about 10-12 cm long, starting at about 0.30 cm from the distal-most end of the hypotube 215 and ending at about 12 cm from the distal-most end. The second section 242 of cuts 232 can be about 9-11 cm long, starting at about 12 cm from the distal-most end of the hypotube 215 and ending at about 21 cm. The third section 244 can be about 19-22 cm long, starting at about 21 cm from the distal-most end of the hypotube 215 and ending at about 40 cm. The fourth section 246 can be about 60-65 cm long, starting at about 40 cm from the distal-most end of the hypotube 215 and ending at about 103 cm. The first pattern P1 can be about 1.5-2 cm long, starting at about 0.30 cm from the distal-most end of the hypotube 215 and end at just 2 cm from the distal-most end such that the first pattern P1 transitions to the second pattern P2 distal to where the first section 240 transitions to the second section 242 of cuts. The second pattern P2 in the embodiment of FIG. 3D can be about 90 cm long, starting at about 2 cm from the distal-most end of the hypotube 215 such that the second pattern P2 continues for at least a portion of section 240, all of section 242 and 244, and at least a portion of the fourth section 246 before it transitions to a third pattern P3 along the length of the fourth section 246. The third pattern P3 can be about 10 cm long, starting at about 93 cm from the distal-most end of the hypotube 215 and ending at about 103 cm.

Still further, FIG. 3G illustrates an implementation where the transitions in pattern P1, P2, P3, P4, P5 are each offset from the transitions in pitch between sections 240, 242, 244, 246, 248. The first section 240 can be about 4-5 cm long, starting at about 0.36 cm from the distal-most end of the hypotube 215 and ending at about 5 cm from the distal-most end. The second section 242 can be about 4-5 cm long, starting at about 5 cm from the distal-most end of the hypotube 215 and ending at about 9 cm. The first pattern P1 is longer than the first section 240 and transitions midway along the second section 252. The third section 244 can be about 12 cm long, starting at about 9 cm from the distal-most end of the hypotube 215 and ending at about 21 cm. The third pattern P3 can be relatively short compared to third section 244 and transitions from the second pattern P2 to the third pattern P3 and the third pattern P3 to the fourth pattern P4 can each occur within the third section 244. The fourth section 246 can be about 18-20 cm long, starting at about 21 cm from the distal-most end of the hypotube 215 and ending at about 40 cm. The fifth section 246 can be about 60-65 cm long, starting at about 40 cm from the distal-most end of the hypotube 215 and ending at about 102 cm. The fourth pattern P4 can be longer than the fourth section 246 and its transitions from the second pattern P3 and to the fifth pattern P5 can be offset from the transitions between sections 244, 246, 248.

Not every transition in pattern need be offset from the transitions in pitch sections. For example, in FIG. 3E, the first section 240 can be about 2-3 cm long, starting at about 0.36 cm from the distal-most end of the hypotube 215 and ending at about 2.5 cm from the distal-most end. The second section 242 can be about 17-19 cm long, starting at about 2.5 cm from the distal-most end of the hypotube 215 and ending at about 21 cm. The third section 244 can be about 18-20 cm long, starting at about 21 cm from the distal-most end of the hypotube 215 and ending at about 40 cm. The fourth section 246 can be about 60-65 cm long, starting at about 40 cm from the distal-most end of the hypotube 215 and ending at about 102 cm. The first pattern P1 transitions to the second pattern P2 at the same location as the first section 240 transitions to the second section 242. The transition between P2 and P3, however, is offset and occurs within the second section 242. Similarly, the transition between P3 and P4 occurs within the fourth section 246. The geometry, patterns, and lengths of the cut regions allow for the hypotube 215 to bend in a first direction along a first radius of curvature to curve through the aortic arch and bend in a second direction along a second radius of curvature to curve away from the aortic arch into at least the common carotid artery/brachiocephalic take-off from the femoral artery access site.

As mentioned above, the reinforcement layer in the proximal portion 210 of the catheter 200 can be a hypotube 215 and the reinforcement layer in the distal portion 205 of the catheter 200 can be a coil reinforcement 220. The distal end of the hypotube 215 (e.g., distal to segment 240 shown in FIGS. 3D-3G) is designed to couple to the proximal end region of the coil reinforcement 220 (see FIG. 3C). In an implementation, the distal end of the hypotube 215 incorporates at least one, preferably 2, 3, 4, or 5 connecting spirals 255 that can intersperse with corresponding connecting spirals 225 at the proximal end region of the coil reinforcement 220 of the distal portion 210 of the catheter 200. The size of the gap between the connecting spirals 255 of the hypotube 215 reinforcement can be substantially similar to the size of the gap between the connecting spirals 225 of the coil reinforcement 220 such that the connecting spirals 225, 255 neatly intersperse with one another without creating any localized areas of increased wall thickness due to overlap. The thickness of the connecting spirals 255 are preferably similar to the thickness of the connecting spirals 225 forming the coil reinforcement 220. For example, the coil reinforcement 220 can be formed of a Nitinol ribbon having a thickness of about 0.003″. The hypotube 215 can have a wall thickness that is about 0.003″-0.004″ such that the connecting spirals 255 at the distal end of the hypotube 215 and the connecting spirals 225 at the proximal end of the coil reinforcement 220 are similar in material thickness. This similarity in material thickness between the spirals 225 of the coil reinforcement 220 and the spirals 255 of the hypotube 215 contribute to a generally uniform outer profile that can be kept to a minimum and avoid creating a substantially increased wall thickness in this coupling region. A low-profile maximizes the inner diameter of the catheter while keeping the outer diameter as small as possible, for example. In an implementation, the coil reinforcement 220 has a thickness of about 0.004″ and 5 turns of coils that are 0.010″ wide and have a pitch of about 0.027″.

The hypotube 215 at the proximal end region can be solid without any cuts 232 or can incorporate a plurality of cuts 232 while also providing sufficient stiffness that allows for advancement of the catheter 200 into distal anatomy without risk of prolapse due to the system 10 lacking a guide sheath. The distal end region of the hypotube 215 is preferably cut to provide transition in stiffness towards the flexibility of the distal portion reinforcement. The flexibility of the distal end region of the hypotube 215 allows for the hypotube to take the curves of the aortic arch and the brachiocephalic take-off from the descending aorta. The metal hypotube preferably avoids entering the skull at significant lengths and transitions to the coil reinforcement 220 so that the distal portion 210 of the catheter 200 can navigate the tortuous vessels of the carotid siphon and beyond that are locked in by the bones of the skull.

Tip flexibility of catheters 200 incorporating laser cut hypotube 215 and multiple transitions in flexibility can be measured by assessing the force in Newtons (N) generated upon deflecting the catheter a certain distance using a particular gauge length. The testing system can include a pin forming a fixed point, an anvil connected by a lever to a strain gauge forming a gauge point. The pin can hold the specimen to be tested such that a gauge length of the specimen is exposed. The anvil attached to the strain gauge via the lever can be urged against a portion of the specimen to be tested that is positioned away from the pin by the gauge length. The anvil displaces this portion such that the portion triggers a force measurable by the strain gauge. The gauge length can be about 5 mm. The anvil can have a width, for example, of about 2 mm, resulting in a minimum gauge length of about 3 mm. The length can vary depending on the testing system.

The bending stiffness of the tip (Elastic modulus×area moment of inertia) can be calculated according to the equation EI=FL³/3δ, where F is deflection force, L is gauge length, and δ is deflection. For example, using a 3 mm gauge length (L=3 mm) and deflecting a tip of the catheter 2 mm (δ=2 mm) within about 10 mm, preferably within about 5 mm, away from the distal end of the catheter, about 0.25 N-0.80 N of force is generated, or about 0.30 N-0.75 N of force, or about 0.35 N-0.70 N of force. In another implementation, the catheter 200 has a total working length of about 115 cm-125 cm and the anvil urged against the catheter within about 5 mm from the distal end generated about 0.35 N-0.45 N of force.

Tip flexibility testing described above assesses the flexibility of the catheter at or near its tip. Three-point bend testing is used to assess bending flexibility along the length of the catheter thereby providing an overall flexibility profile for the catheter. The three-point bend test involves gripping the catheter at two points that are a fixed distance apart (e.g., 3 cm) and assessing the force in Newtons (N) generated upon deflecting the catheter a certain distance with an anvil positioned centrally on the catheter between the two points. The bending flexibility of the catheters described herein are listed in Table 1 below.

TABLE 1

| Catheter | Distance from | |
|---|---|---|
| length | Proximal end | Force |
| (cm) | (cm) | (N) |
| 115 | 20-60 | 13.0-14.4 |
| | 80-86 | 8.9-11.6 |
| | 90-98 | 0.71-2.0 |
| | 102-112 | 0.63-0.70 |
| 125 | 20-70 | 13.0-14.4 |
| | 80-96 | 8.9-11.6 |
| | 90-108 | 0.71-2.0 |
| | 102-122 | 0.63-0.70 |

As discussed above, the catheters 200 described herein have a tubular catheter body 202 with a proximal portion 205 and a distal portion 210. The hypotube 215 reinforces the proximal portion 205 and the coil 220 reinforces the distal portion 210. The proximal end region of the hypotube 215 can be solid without any cuts or can include a plurality of cuts 232 while also providing sufficient stiffness that allows for advancement of the catheter 200 into distal anatomy without risk of prolapse due to the system 10 lacking a guide sheath. For a catheter that is about 115 cm-125 cm, the hypotube 215 can extend about 90-115 cm from the distal-most end of the strain relief and at least about 90%-100% of the hypotube 215 can incorporate cuts 232 for flexibility where the pattern, pitch, and overall arrangement of the cuts 232 is selected to provide a transition in flexibility over its length. For example, the proximal end region of the catheter reinforced by the hypotube 215 (i.e., proximal section) has a bending stiffness as measured by three-point bend test of about 11-18 N, or about 13-15 N. In contrast, the distal portion 210 of the catheter that is reinforced with the coil 220 and is about 20-30 cm from the distal end of the catheter 200 (i.e., distal section) has a bending stiffness of less than 2 N, preferably less than 1 N. The catheters 200 described herein have a transition region between the stiffer proximal portion 205 reinforced by the hypotube 215 and the very flexible distal portion 210 reinforced by the coil 220. As discussed above with regard to FIG. 3C, there is a transition between the distal end of the hypotube 215 and the proximal end of the coil 220 where respective coils of each 225, 255 intersperse or interlock with one another. The location of these interlocking coils, depending on the overall length of the catheter, is about 85-90 cm from the proximal strain relief. This location is also where the hypotube 215 changes significantly in flexibility, in part, due to the spiral cuts forming the coils 255, and also due to the cuts 232 formed into various geometries, patterns, and lengths of the cut segments (i.e., transition section). Thus, the catheter 200 includes a proximal section, a distal section, and a transition section therebetween, each contributing to the bending stiffness profile. The proximal section and transition section each reinforced by the hypotube 215 and the distal section 215 not reinforced by the hypotube.

The transition section undergoes a large change in bending stiffness that forms a bridge between the high bending stiffness of the proximal section and the low bending stiffness of the distal section. The transition section of the catheter having a working length of about 115 cm-125 cm extends less than about 30 cm, preferably less than 20 cm, and changes in bending stiffness along that length from about 10-20 N near the proximal section to about 1-2 N near the distal section. The difference between the distal section bending stiffness and the proximal section bending stiffness can be at least 10×, preferably at least 15×. The difference in bending stiffness along the transition section can be at least 4×, and preferably at least 5× from one end of the transition section to the other. The total change in bending stiffness along the entire length of the catheter 200 (from strain relief to distal-most tip), particularly for the 115-125 cm working lengths, can be greater than about 10×, preferably greater than about 12×. The proximal section (i.e., within 50-70 cm of strain relief of catheter) can have a bending stiffness that is greater than about 12 N, preferably greater than about 13 N, and most preferably greater than about 14 N. The change in stiffness in the proximal section of the catheter is less than the change in stiffness in the transition section of the catheter.

The bending stiffness profile of the catheter 200 renders it capable of being advanced through a patient's vasculature to the neurovasculature without being positioned inside of another catheter, except for an introducer sheath at the access site location. Introducer sheaths are typically relatively short and often have a length less than about 30 cm such that for a catheter 200 introduced via the femoral artery or even a radial artery, they provide little to no support in the catheter's ability to access intracranial vessels. The bending stiffness profile of the catheter 200 allows the distal section of the catheter 200 to reach the middle cerebral artery without the proximal section of the catheter 200 prolapsing, such as into a region of the aorta (e.g., ascending aorta).

The pitch of the cuts 232, the pattern of cuts 232 (i.e., amount of the hypotube wall that is cut versus uncut), and to some degree the shore hardness of the polymer segments of the outer jacket layer overlaying the hypotube 215, contribute to the transition in flexibility of the catheter 200 from the hypotube-reinforced proximal end to the coil-reinforced distal end. The outer jacket layer has a bigger impact on the degree of flexibility achieved in the last 20-30 cm of the distal portion 210 reinforced without the hypotube, but rather is coil-reinforced. As an example, a first segment of the outer jacket layer can be formed of PEBAX Shore 62A and extend a length of about 55-65 mm from a distal-most end. A second segment of the outer jacket layer proximal to the first segment can be formed of PEBAX Shore 80A and extend a length of about 75 mm-85 mm. Moving proximally from the second segment, a third segment of the outer jacket layer can be formed of PEBAX Shore 85A and extend a length of about 25-35 mm. Moving proximally from the third segment, a fourth segment of the outer jacket layer can be formed of PEBAX Shore 25D and extend a length of about 25-35 mm. Moving proximally from the fourth segment, a fifth segment of the outer jacket layer can be formed of PEBAX Shore 35D and extend a length of about 25-35 mm. Moving proximally from the fifth segment, a sixth segment of the outer jacket layer can be formed of PEBAX Shore 45D and extend a length of about 25-35 mm. Moving proximally from the sixth segment, a seventh segment of the outer jacket layer can be formed of PEBAX Shore 55D and extend a length of about 25-35 mm. Moving proximally from the seventh segment, an eight segment of the outer jacket layer can be formed of PEBAX Shore 72D and extend a length of about 55-65 mm. Moving proximally from the eighth segment, a ninth segment of the outer jacket layer can be formed of Nylon and extend a length of about 860-950 mm.

For a catheter 200 having a working length of about 110-135 cm, the hypotube 215 can be about 85-120 cm long. At least the eighth and the ninth segments of the outer jacket layer 207 and a portion of the seventh segment overlay the hypotube 215. The remaining outer jacket layers overlay the coil-reinforced distal portion 210 without the hypotube 215. Where the outer jacket layer 207 overlays the cuts 232 of the hypotube 215, the polymer material of the outer jacket layer 207 can flow into the cuts 232 so the polymer bonds with the inner liner layer 203 internal to the hypotube 215 (or the coil 220). In an implementation, the outer diameter of the inner liner layer 203 is about 0.0908″ and the inner diameter of the hypotube 215 is about 0.0950″ having a wall thickness of about 0.0040″. The polymer of the outer jacket layer 207 can flow through the 0.004″ depth cuts 232 in the wall of the hypotube 215 into the radial clearance between the hypotube 215 and the inner liner layer 203, which would be about 0.002″ or 0.0042″/2.

The low bending stiffness distal section, which includes a liner layer, a coil reinforcement layer, and the outer jacket layer, can include at least 4 transitions in jacket durometer. The 4 transitions in outer jacket durometer can occur over about 20-30 cm. The high bending stiffness proximal section, which includes the liner layer, the hypotube reinforcement, and the outer jacket layer, can include at least 2 transitions in cut pattern and 2 transitions in jacket durometer. The 2 transitions can occur over about 70-80 cm. The transition section that undergoes the large change in bending stiffness between the high bending stiffness of the proximal section and the low bending stiffness of the distal section, and which includes the liner layer, the hypotube reinforcement, and the outer jacket layer, can include at least 4 transitions in cut pattern and/or pitch. The 4 transitions can occur over less than about 30 cm, preferably less than about 20 cm. This transition section, which can be about 85-90 cm from the proximal strain relief, can undergo a steep drop in bending stiffness (e.g., 12N to IN) over a relatively short distance (e.g., 10 cm) to help maintain a smooth transition in flexibility along the length of the catheter 200 that prevents kinking. The outer jacket layer in the proximal portion of the catheter has a higher durometer segment than a durometer of a segment of the outer jacket layer in the distal portion. The higher durometer segment of the outer jacket layer in the proximal portion can be greater than about 55D. The durometer of the segment of the outer jacket layer in the distal portion can be less than about 25D. The outer jacket layer can include a plurality of segments that transition in durometer between a durometer greater than about 55D and a durometer less than or equal to about 25D, where the plurality of segments is at least 5 segments.

In some implementations, the catheter 200 has a shorter working length and a different series of transitions in the hypotube to the distal coil of the distal portion. A catheter having a total working length of about 95 cm can generate about 0.50 N-0.70 N of force in the tip flexibility testing when the anvil is urged against the catheter within about 5 mm from the distal end.

The catheter having the shorter working length also has a different bending flexibility profile as assessed by three-point bend testing. For a catheter of this length, the hypotube 215 can extend about 70-90 cm from the distal-most end of the strain relief and at least about 90%-100% of the hypotube 215 can incorporate cuts 232 for flexibility where the pattern, pitch, and overall arrangement of the cuts 232 is selected to provide a transition in flexibility over the length of the hypotube. For example, the proximal end region of the catheter reinforced by the hypotube 215 extending about 20-70 cm from the proximal strain relief (i.e., proximal section) can have a bending stiffness as measured by three-point bend test of about 11.8 N-15.1 N. The distal portion 210 of the catheter that is reinforced with the coil 220 and extends about 90-95 cm from the proximal strain relief (i.e., distal section) can have a bending stiffness of about 1.84 N-5.0 N. The region of the catheter reinforced by the hypotube 215 proximal of the distal section and extending about 80-86 cm from the proximal strain relief (i.e., transition section) can have a bending stiffness of about 7.2 N-8.0 N.

### Distal Portion Reinforcement

The reinforcement layer of the distal portion 210 is a generally tubular coil reinforcement 220 formed of, for example, a wound ribbon or wire (see FIGS. 3B-3C). The coil reinforcement 220 extends between the inner liner layer 203 and the outer jacket layer 207 within the distal portion 210 of the catheter body 202. The proximal end region of the coil reinforcement 220 is engaged with the distal end region of the hypotube 215 as described above and the distal end region of the coil reinforcement 220 terminates near a distal end 206 of the catheter body 202. In some implementations, the coil reinforcement 220 terminates near a location of a distal marker band 211 (see FIG. 3B).

The material for the coil reinforcement 220 may be stainless steel, for example 304 stainless steel, Nitinol, cobalt chromium alloy, or other metal alloy that provides the desired combination of strengths, flexibility, and resistance to crush. In some implementations, the reinforcement structure is a Nitinol ribbon wrapped into a coil. For example, the coil reinforcement 220 can be a tapered ribbon of Nitinol set to a particular inner diameter (e.g., 0.085″ to 0.095″ inner diameter) and having a pitch, which can increase from proximal end of the coil reinforcement 220 towards distal end 206 of the catheter body 202 (e.g., about 0.025″ to about 0.028″ near a proximal end region and about 0.034″ to about 0.036″ near a distal end region). The ribbon can be 304 stainless steel (e.g., about 0.012″×0.020″). The coil reinforcement 220 can be heat-set prior to transferring the coil reinforcement 220 onto the catheter. For example, the ribbon coils can have gaps in between them, and the size of the gaps can increase moving towards the distal end of the distal portion 210. For example, the size of the gap between the ribbon coils can be approximately 0.027″ gap near the proximal end of the distal portion 210 and the size of the gap between the ribbon coils near the distal end can be larger such as 0.035″ gap. This change in pitch provides for increasing flexibility near the distal-most end of the distal portion 210.

The carotid siphon CS is an S-shaped part of the terminal ICA beginning at the posterior bend of the cavernous ICA and ending at the ICA bifurcation into the anterior cerebral artery ACA and middle cerebral artery MCA (see FIG. 1A). The distal portion 210 of the catheter 200 can have a length measured from the transition between the hypotube 215 to the coil reinforcement 220 to the distal end 206 of the catheter body 202 that is long enough to extend from a region of the internal carotid artery (ICA) that is proximal to the carotid siphon CS to a region of the ICA that is distal to the carotid siphon (CS), including at least the M1 region of the brain. As discussed above, the proximal portion 205 can be about 70 cm to about 110 cm long, preferably about 80 cm to about 100 cm. The distal portion 210 can be between about 20 cm to about 50 cm, about 25 cm to about 35 cm long, or about 28 cm to about 30 cm long, to allow for the distal end of the catheter 200 to extend into at least the middle cerebral arteries while the distal end region of the proximal hypotube 215 extends distal to the brachiocephalic take-off and proximal to the carotid siphon CS. In some implementations, the hypotube 215 has a length sufficient to allow the distal end region of the hypotube 215 to be positioned within about 20 cm, within about 25 cm, within about 30 cm away from an occlusion located in an intracranial vessel (e.g., distal to the carotid siphon within the M1 or M2 level of the cerebral vasculature). The lengths of the portions 205, 210, and the catheter 200 length overall can vary depending on the point of insertion for the catheter 200, including femoral, carotid, radial, brachial, ulnar, or subclavian arteries. The lengths of the body 202 described herein can be modified to accommodate different access points. For example, a catheter body 202 of a catheter 200 for entry through the femoral artery near the groin may be longer than a catheter body 202 of a catheter 200 for entry through the subclavian artery.

The combination of lengths can be selected to ensure the hypotube 215 of the proximal portion 205 remains outside the skull and the coil reinforcement 220 of the distal portion 210 navigates the intracranial vessels. Again with respect to FIGS. 1A-1D, the brachiocephalic take-off (BT) is typically a very severe turn off the aortic arch AA for a transfemorally-delivered catheter seeking the right-sided cerebral circulation. A catheter traversing from the femoral artery through the iliac circulation into the descending aorta DA turns as it approaches the aortic arch AA and reaches across the take-off of other great vessels to reach the brachiocephalic take-off (BT), which is the furthest “reach” of the great vessels of the aortic arch AA. FIG. 1D shows the substantial and obligatory S-turn created by that anatomy. A catheter must traverse this S-turn along a path of insertion from a femoral artery insertion location in order to reach the internal carotid artery (ICA). The left ICA often takes off from the brachiocephalic and thus, has a similar challenge and can create an even tighter S-turn. Should the left ICA have a typical take-off between the brachiocephalic BT and the left subclavian artery LSA take-off, then the reach may be less severe, but an S-turn still develops of lesser severity. The catheter 200 can be provided in different lengths, for example, a medium length catheter 200 that is about 115 cm, and a longer catheter 200 that is about 125 cm for access of the M1. Each can incorporate a hypotube 215 with stiffer proximal section and more flexible distal section. The catheter 200 can be designed so the entire hypotube 215 remains outside the skull or only the more flexible distal section of the hypotube 215 enters the skull while the stiffer proximal section of the hypotube 215 remains outside the skull. The catheters 200 having different overall working lengths can incorporate a hypotube having the same pattern and lengths or can vary.

In some implementations, the length of the distal portion 210 is sufficient to reach a region of the M1 segment of the middle cerebral artery (MCA) and other major vessels from a region of the internal carotid artery while the uncut portion of the hypotube 215 in the proximal portion 205 of the catheter 200 is located proximal to certain tortuous anatomies (e.g., within the descending aorta DA) when advanced from an access site in the groin. In an implementation, the distal portion 210 of the catheter has a length sufficient to position its distal end 206 within the M1 segment of the MCA, the cut portion of the hypotube 215 traverses the aortic arch to a region distal to the brachiocephalic take-off, and the uncut portion of the hypotube 215 is located within the descending aorta when advanced from an access site in the groin. In an implementation, the distal portion 210 of the catheter has a length sufficient to position its distal end 206 within the M1 segment of the MCA, the cut portion of the hypotube 215 traverses the aortic arch to a region distal to the brachiocephalic take-off, and the uncut portion of the hypotube 215 is located within the descending aorta DA proximal to the aortic arch AA when advanced from an access site in the groin. The cut portion of the hypotube 215 can incorporate a pattern of cuts, slots, interruptions, or other features can be flexible enough to enter the aortic arch and the brachiocephalic artery up into the internal carotid so that the coil reinforcement 220 of the distal reinforcement need not be as long. The access site can vary including the groin (femoral), carotid, radial, brachial, ulnar, or subclavian arteries.

The coil reinforcement 220 can extend a length along the distal portion 210, for example, about 20-30 cm. The portion of the coil reinforcement 220 that has a first narrower pitch for less flexibility can be a first length of the total length of the coil reinforcement 220 and the portion of the coil reinforcement 220 that has a second wider pitch for more flexibility can be a second length of the total length of the coil reinforcement 220. As an example, the length of the proximal coil pitch can be about 25 cm to about 28 cm and the length of the distal coil pitch can be about 2 cm to about 5 cm. The ratio of proximal coil pitch segment length to distal coil pitch segment length can vary including 1:1, 2:1, 3:1, 4:1, 5:1, 6:1, 7:1, 8:1, 9:1, 10:1, 11:1, 12:1, 13:1, 14:1 and the like.

The reinforcement layer is described as being a hypotube 215 in the proximal portion 205 and a coil reinforcement 220 in the distal portion 210, however, the configuration and material of the reinforcement layer can vary. The reinforcement layer can be made from metal such as 304 stainless steel, Nitinol, Nitinol braid, helical ribbon, helical wire, cut stainless steel, cobalt chromium alloy, or other metal alloy, or the like, or stiff polymer such as PEEK. The reinforcement layer can be laser-cut, etching, sanding, polishing, or machine-cut so as to be flexible in a distal end region as discussed above. The reinforcement layer can be made from a “removal” process in which materials are removed from a raw tube to create openings (e.g., cuts 232) through a wall of the tube. The reinforcement layer can provide a desired combination of strength, flexibility, and resistance to crush.

The reinforcement layer can also vary so that combinations of reinforcement are provided along at least a region of the catheter. As an example, a proximal portion 205 of the catheter 200 can include a braid to provide good torqueability and the braid can be overlaid by a coil to provide good kink resistance. As another example, the proximal portion 205 of the catheter 200 can include a hypotube to provide good pushability that incorporates interruptions, cut-outs, or other feature at its distal end region to provide increasing flexibility towards the distal end region of the catheter 200.

The catheter body 202 can additionally incorporate one or more reinforcement fibers 250 configured to prevent elongation of the coil reinforcement 220 (see FIG. 3B). The reinforcement layer (e.g., coil structure 220 and/or reinforcement fiber 250) can be applied to the inner liner 203, followed by the outer jacket layer 207 and/or additional outer coating prior to removing the mandrel by axial elongation. The fiber 250 can be positioned between the liner layer 203 and the reinforcement layer 215, 220. The fiber 250 can extend along the longitudinal axis A of the catheter 200 from a proximal end region to a distal end region. In some implementations, the proximal end of the fiber 250 can be coupled to a distal end region of the hypotube 215 near where it couples to the coil reinforcement 220. In other implementations, the fiber 250 extends along the entire working length of the catheter body 202 including between the hypotube 215 and the liner 203 and between the coil reinforcement 220 and the liner 203. Where the fiber 250 extends only through coil-reinforced regions, the hypotube 215 can include an aperture or other feature that is configured to receive a proximal end of the reinforcement fiber 250 extending longitudinally through the catheter portion.

A distal end of the fiber 250 can terminate near the distal end 206 of the catheter. The distal end of the fiber 250 can be captured between the distal marker band 211 and an end of the coil reinforcement 220 (see FIG. 3B). The distal marker band 211 can be fully encapsulated between the inner liner 203 and the outer jacket 207. In some implementations, the distal end of the fiber 250 extends distal to the last spiral 225 of the coil reinforcement 220 looping around the spiral 225 back in a proximal direction running under the marker band 211. The free end of the fiber 250 is thereby captured between the coil reinforcement 220 and the marker band 211. The reinforcement fiber 250 thus terminates at the location the reinforcement layer 220 terminates thereby leaving a length of between about 1.0 mm-0.5 mm of the distal end region of the catheter body 202 that has no coil reinforcement 220. The catheter 200 can include a plurality of reinforcement fibers 250 extending longitudinally along the catheter body 202, such as two, three, four, or more fibers 250 distributed around the circumference of the catheter body 202 and aligned parallel with one another and with the longitudinal axis A of the catheter 200. The reinforcement fiber(s) 250 may also terminate at a more distal location or at a more proximal location than the location of the distal spiral 225 of the coil reinforcement 220. The material of the reinforcement fiber 250 can vary, including but not limited to various high tenacity polymers like polyester, PEEK, and other similar materials.

The inner liner 203 can be constructed, as an example, from a low friction polymer such as PTFE (polytetrafluoroethylene) or FEP (fluorinated ethylene propylene) to provide a smooth inner surface. The lubricious inner liner 203 can have one or more thicknesses along variable sections of flexibility. The PTFE liner can be a tubular liner formed by dip coating or film-casting a removable mandrel, such as a silver-plated copper wire mandrel. Various layers can be applied having different thicknesses. For example, a base layer of etched PTFE can be formed having a thickness of about 0.005″. A second, middle layer can be formed over the base layer that is Tecoflex SG-80A having a thickness of about 0.0004″. A third, top layer can be formed over the middle layer that is Tecoflex SG-93A having a thickness of about 0.0001″ or less. The inner liner 203 can extend an entire working length of the catheter body 202. FIG. 3B shows the inner liner 203 extending to the distal-most end of the catheter body 202 distal to the marker band 211 and distal to the coil reinforcement 220 and the fiber 250.

As mentioned above, the flexibility of the body 202 can vary over its length, with increasing flexibility towards the distal portion 210 of the body 202. The distal portion 210 of the catheter 200 is constructed to be flexible and lubricious to safely navigate to the target location. The distal portion 210 is kink resistant and collapse resistant when subjected to high aspiration forces so as to be able to effectively aspirate a clot. The distal portion 210 has increasing flexibility towards the distal end with smooth material transitions along its length to prevent any kinks, angulations or sharp bends in its structure, for example, during navigation of severe angulations such as those having 90° or greater to 180° turns, for example at the aorto-iliac junction, the left subclavian take-off from the aorta, the takeoff of the brachiocephalic (innominate) artery from the ascending aorta and many other peripheral locations just as in the carotid siphon. The distal portion 210 transitions from being less flexible near the junction with the proximal portion 205 (i.e., where the reinforcement components connect) to being more flexible at the distal-most end. The change in flexibility from proximal to distal end of the distal portion 210 can be achieved by any of a variety of methods as described herein.

The outer jacket layer 207 is formed of materials that provide mechanical integrity to the inner liner layer 203, for example, materials such as PEBAX, thermoplastic polyurethane, polyethylene, nylon, or the like. The outer jacket layer 207 may be composed of discreet sections of polymer with different durometers, composition, and/or thickness to vary the flexibility along the length of the catheter body 202. A lower durometer outer jacket material can be used in the distal portion 210 of the catheter compared to the proximal portion 205 of the catheter 200. The proximal portion 205 of the catheter can incorporate outer jacket material such as Nylon. The Nylon outer jacket material can transition moving distally within the proximal portion 205 to an outer jacket material having a lower durometer, for example, 72D PEBAX. The 72D PEBAX material can transition moving distally within the proximal portion 205 to an outer jacket material having a lower durometer, for example, 55D PEBAX or a combination of 72D and 55D PEBAX. The outer jacket layer 207 can transition to increasingly more flexible materials having a hardness of 55D PEBAX, 45D PEBAX, 35D PEBAX, 25D PEBAX, 85A Tecoflex, 80A Tecoflex, 65A Tecothane, 62A Tecothane, and combinations thereof approaching the distal end 206. The distal end 206 can be formed of a material having a hardness of no more than about 62A Tecothane, no more than about 80A Tecoflex, or no more than about 85A Tecoflex, or no more than about 35D PEBAX. For example, a first region of the catheter 200, such as a region of the proximal portion 205, can be formed of a material having a hardness of at least about 72D or greater along a first length, a second region of the catheter 200, such as a region of the proximal portion 205 and/or the distal portion 210, can be formed of a material having a hardness that is less than about 72D, such as about 55D along a second length, a third region of the catheter 200 can be formed of a material having a hardness that is less than about 55D, such as about 35D along a third length, a fourth region of the catheter 200 can be formed of a material having a hardness less than about 35D, such as about 25D along a fourth length, a fifth region of the catheter 200 can be formed of a material having a hardness less than about 25D, such as about 85A Tecoflex along a fifth length, a sixth region of the catheter 200 can be formed of a material having a hardness less than about 85A Tecoflex, such as about 80A Tecoflex along a sixth length, a seventh region of the catheter 200 can be formed of a material having a hardness less than about 80A Tecoflex, such as about 62A Tecothane. In some implementations, the final distal region of the catheter 200 can be formed of a material such as Tecothane having a hardness of 62A that is matched in hardness to a region of the delivery catheter 300, which will be described in more detail below. The distal portion 210 transitions from being less flexible near its junction with the proximal portion 205 to being more flexible at the distal-most end where, for example, a distal tip of the delivery catheter 300 extends distal to the distal-most end. The lengths of each of the segments of outer jacket 207 can vary. In some implementations, such as the Nylon segment at a proximal end region of the catheter, the segment can extend along a length of about 80 cm-110 cm. The regions of the catheter having a hardness of about 72D, 55D, 45D, 35D, 25D, 85A, 80A, 65A, 62A and the like, can have any of a variety of lengths between about 30 mm and about 80 mm. The wall thickness of the outer jacket material can be uniform along the length of the catheter body 202 or may be reduced moving distally along the catheter 200.

The material hardnesses described herein with respect to the catheter 200 can be achieved by a single polymer material or by mixtures of polymer materials. For example, a mixture of 35D PEBAX and 55D PEBAX can provide a harder polymeric material than that of 35D PEBAX alone and a softer polymer material than that of 55D PEBAX alone. The polymer segments of the various catheter components described herein can incorporate any of a variety of hardnesses between the specific hardnesses identified by blending of one or more polymer materials to achieve a transition in flexibility along a length of the structure. Additionally, the ranges of hardnesses between the proximal end portions of the catheters described herein and a distal end portions of the catheters can vary from greater than 72D PEBAX (e.g., 72D PEBAX reinforced with a metallic or non-metallic element) down to less than 35D (e.g., 62A Tecothane).

The changes in material hardness of the outer jacket 207, in combination with the changes in density of the reinforcement layer, may be varied to increase the flexibility moving distally along the catheter 200. As discussed above, the distal portion 205 of the catheter 200 incorporates the coil reinforcement 220 and the pitch of the coil reinforcement 220 may be stretched out at the distal end region compared to more proximal areas of the coil reinforcement 220. The cut pattern in the hypotube 215 of the proximal portion 205 may be varied to be more flexible near the distal end where the hypotube 215 connects to the proximal end of the coil reinforcement 220 of the distal portion 210. The structure of the reinforcement layer (i.e., hypotube to coil) and/or the materials of the reinforcement layer may change over the length of the catheter body 202. In another implementation, there is a transition section between the more flexible distal portion 205 and the stiffer proximal portion 210, with one or more sections of varying flexibilities between them.

It is desirable that the catheter 200 have an inner diameter that is as large as possible that can be navigated safely to the site of the occlusion, in order to maximize the aspiration force in the case of aspiration and/or provide ample clearance for delivery of a working device. A suitable size for the inner diameter of the catheter body 202 (i.e., inner diameter of lumen 223) is between 0.054″ and 0.108″, or more preferably between 0.070″ and 0.095″, most preferably between 0.088″ and 0.090″, depending on the patient anatomy and the clot size and composition. The working lumen can have an inner diameter that is at least about 0.054″, at least about 0.070″, at least about 0.071″, at least about 0.072″, at least about 0.073″, at least about 0.074″, at least about 0.075″, at least about 0.076″, at least about 0.077″, at least about 0.078″, at least about 0.079″, at least about 0.080″, at least about 0.081″, at least about 0.082″, at least about 0.083″, at least about 0.084″, at least about 0.085″, at least about 0.086″, at least about 0.087″, at least about 0.088″, at least about 0.089″, at least about 0.090″, at least about 0.100″, up to about 0.108″. The inner diameter of the catheter lumen 223 can be substantially similar along an entire working length of the catheter body 202 so that the inner diameter of the distal portion 210 including the distal opening 208 of the catheter 200 has the same or similar inner diameter as the inner diameter of the proximal portion 205 of the catheter 200. The inner diameter of the catheter lumen 223 can gradually increase moving proximally along the catheter body 202 to be larger at the proximal end of the catheter or can incorporate a step-up in inner diameter at a point along the length.

It is desirable that the catheter 200 have an outer diameter that allows for the large inner diameter to draw aspiration forces and/or deliver interventional tools. The outer diameter of the distal portion 210 can be sized for navigation into cerebral arteries, for example, at the level of the M1 segment or M2 segment of the cerebral vessels. The outer diameter should be as small as possible while still maintaining the mechanical integrity of the catheter 200. As discussed above, the proximal portion 205 of the catheter 200 is designed to provide sufficient proximal support to avoid prolapse even without the presence of a guide catheter or sheath. Because the catheter 200 needs no proximal guide sheath, the proximal outer diameter is smaller than what would otherwise be necessary for a telescoping catheter system. The smaller proximal outer diameter avoids enlarging the arteriotomy without compromising the large inner diameter at the distal opening 208.

A suitable size of the outer diameter of the catheter body 202, particularly the proximal portion 205 of the catheter, is between 0.065″ and 0.110″, or more preferably between 0.080″ and 0.105″, most preferably between 0.098″ and 0.102″, depending on the size of the inner lumen. The outer diameter of the catheter body 202 is preferably smaller than an outer diameter of a long guide sheath. For example, an outer diameter of an 8F size guide sheath can be about 0.136″/3.45 mm. The outer diameter of the catheter body 202 is preferably no greater than 0.122″/3.1 mm to ensure the size of the arteriotomy is minimized, even where the inner diameter is relatively large. The outer diameter of the catheter 200 can be uniform along the proximal portion 205 and along the distal portion 210 such that the catheter body 202 has substantially a single outer diameter along its working length, for example, about 0.089″ to about 0.102″ outer diameter. In other implementations, the outer diameter of the catheter 200 changes over its length. For example, the distal portion 210 can have a reduced outer diameter compared to the outer diameter of the proximal portion 205, for example, about 0.088″ to about 0.090″ at the distal end 206 compared to about 0.100″ to about 0.102″ at the proximal end. The outer diameter can taper down or step-down from the outer diameter of the proximal portion 205 to the outer diameter of the distal portion 210 over a transition segment. The transition segment can be located near where the hypotube 215 of the proximal portion 215 transitions to the coil reinforcement 220 of the distal portion 210.

Regardless of its inner diameter and length, the catheter 200 is resistant to kinking during distal advancement through the vasculature.

The catheter 200 includes one or more radiopaque markers 211. At least one radiopaque marker 211 is disposed near the distal opening 208 at the distal end 206 of the catheter 200. The radiopaque bands 211 may be swaged, painted, embedded, or otherwise disposed in or on the body 202. The radiopaque markers 211 of the distal portion 210, particularly those near the distal end 206 navigating extremely tortuous anatomy, can be relatively flexible such that they do not affect the overall flexibility of the distal portion 210 near the distal tip region. The marker 211 is shown in the figures as a single ring around a circumference of one or more regions of the body 202. However, the marker 211 need not be a single ring and can include 2 or more rings forming a single marker 211. The marker 211 also need not be a ring and can have other shapes or create a variety of patterns that provide orientation to an operator regarding the position of the distal opening 208 within the vessel. The radiopaque marker(s) 211 can be a barium polymer, platinum or platinum-loaded, gold, tantalum, tungsten, tungsten polymer blend, tungsten-loaded or any other substance visible under an x-ray fluoroscope. In some implementations, the radiopaque marker 211 is a tungsten-loaded PEBAX or polyurethane that is heat welded to the body 202. The band of tungsten-loaded PEBAX may have a durometer of 35D. In other implementations, the radiopaque marker 211 is a platinum-iridium metallic marker band embedded in polymer. The marker can maintain flexibility of the distal end of the device and improve transition along the length of the catheter 200 and its resistance to kinking. Any of the components of the systems described herein can incorporate radiopaque markers 211 as described above.

The proximal end region of the hypotube 215 of the proximal portion 205 of the catheter body 202 can be coupled to a strain relief, which in turn, is coupled to a rotating hemostatic valve (RHV) 204. The RHV 204 can include one or more lumens molded into a connector body to connect to a working lumen 223 of the catheter 200. The RHV 204 can be constructed of thick-walled polymer tubing or reinforced polymer tubing. The RHV 204 allows for the application of aspiration or introduction of devices and the like through the catheter 200, while preventing or minimizing blood loss and preventing air introduction into the catheter 200. The RHV 204 can be integral to the catheter 200 or the catheter 200 can terminate on a proximal end in a female Luer adaptor to which a separate hemostasis valve component, such as a passive seal valve, a Tuohy-Borst valve or rotating hemostasis valve may be attached. The RHV 204 can have an adjustable opening that is open large enough to allow removal of devices that have adherent clot on the tip without causing the clot to dislodge at the RHV 204 during removal. Alternately, the RHV 204 can be removable such as when a device is being removed from the catheter 200 to prevent clot dislodgement at the RHV 204. The RHV 204 can be a dual RHV.

The RHV 204 can form a Y-connector on the proximal end region of the catheter 200 such that the first port of the RHV 204 can be used for insertion of device into the working lumen of the catheter 200 and a second port into RHV 204 can be used for another purpose, such as drawing aspiration through the catheter 200. For example, a syringe or other device can be connected at arm via a connector for delivering a forward drip, a flush line for contrast or saline injections through the body 202 toward the distal end region and into the target anatomy. Arm can also connect to a large-bore aspiration line and an aspiration source, such as a syringe or pump to draw suction through the working lumen. The aspiration source can be an active source of aspiration such as an aspiration pump, a regular or locking syringe, a hand-held aspirator, hospital suction, or the like, configured to draw suction through the working lumen. The aspiration source can be a locking syringe (for example a VacLok syringe) attached to a flow controller. The arm can also allow the catheter 200 to be flushed with saline or radiopaque contrast during a procedure.

One or more regions of the catheter 200 can be coated with a lubricious coating, such as a hydrophilic coating on an inner surface and/or an outer surface. Suitable lubricious polymers include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, HYDAK coatings (e.g., B-23K, HydroSleek), and the like, and mixtures and combinations thereof. Hydrophilic polymers may be blended among themselves or with formulated amounts of water insoluble compounds (including some polymers) to yield coatings with suitable lubricity, bonding, and solubility.

The terms “catheter,” “neurocatheter,” “large bore catheter,” “aspiration catheter,” “interventional catheter,” and the like may be used interchangeably herein. Where the catheter is described herein as an aspiration catheter it should not be limited to only aspiration. Similarly, where the catheter is described herein as a way to deliver a stent retriever or other working devices, it should not be limited as such. It should also be appreciated that the systems described herein can be used to perform procedures that incorporate a combination of treatments. For example, the catheter can be used for the delivery of a stent retriever delivery system, optionally in the presence of aspiration through the catheter. As another example, a user may start out performing a first interventional procedure using the systems described herein, such as aspiration thrombectomy, and switch to another interventional procedure, such as delivery of a stent retriever or implant.

### Navigation Catheters

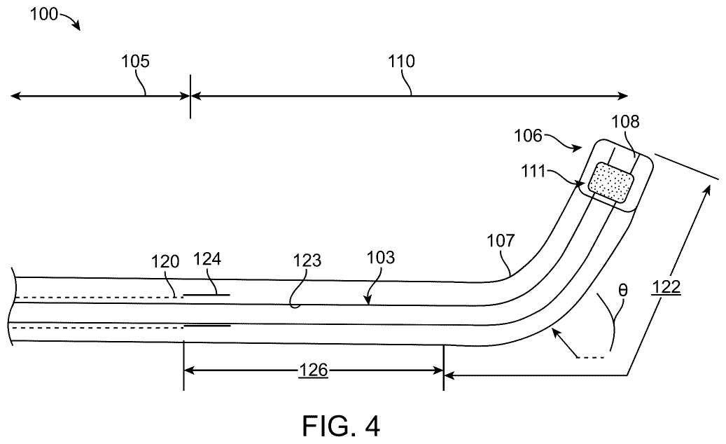

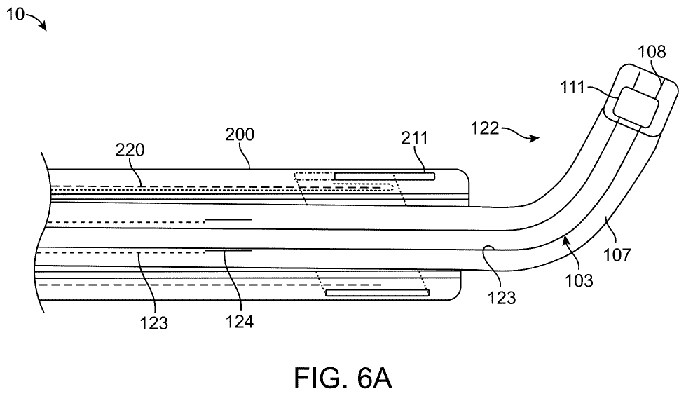

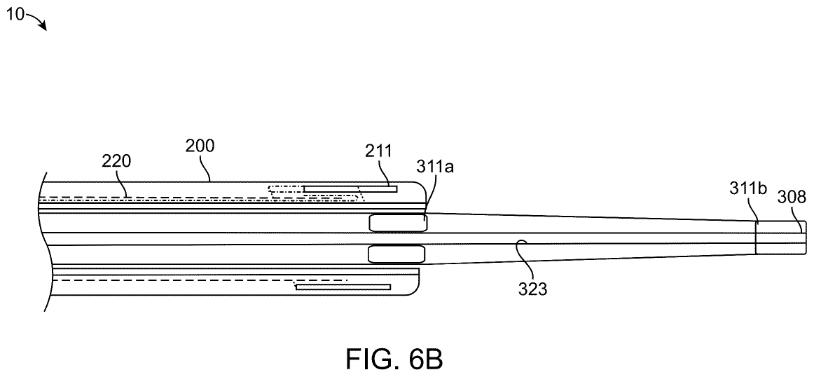

Again with respect to FIG. 2 and also FIGS. 4 and 6A, the catheter systems 10 described herein can include a navigation catheter 100 configured to advance the catheter 200 through a patient's vasculature without the aid of a long guide sheath. The catheter 200 has exceptional deliverability for advancement to the level of the M1 or M2 despite the relatively large inner diameter. The navigation catheter 100 supports the advancement of the catheter 200 to reach distal sites, such as vessels just distal to the aortic arch. For example, the navigation catheter 100 can be used to advance the distal portion 210 of the catheter 200 to a first location. Preferably, the navigation catheter 100 is advanced to a site that is distal of the aortic arch within an internal carotid artery (ICA) and proximal of the carotid siphon CS. As will be discussed in more detail below, the navigation catheter 100 can include a metal reinforcement layer that extends up to about 2 cm away from the distal-most end 206 of the catheter 100. It is preferred that the metal reinforcement layer remain at a location that is outside the skull. Once the navigation catheter 100 advances the catheter 200 to a target location, it is exchanged for the exceedingly flexible, delivery catheter 300 having an unreinforced distal portion 310 configured to aid advancement of the distal portion 210 of the catheter 200 to a second location distal to the first location, such as distal to a petrous portion of the ICA and beyond. The navigation catheter 100 and the delivery catheter 300 will be described in more detail below.

The navigation catheter 100 can include a tubular body 102 defining a single inner lumen 123 having a working length between a proximal end and a distal end of the navigation catheter 100. The tubular body 102 includes a proximal portion 105 that is a relatively stiff shaft that transitions to a more flexible distal portion 110. The tubular body 102 of the navigation catheter 100 is configured to assume and navigate the bends of the vasculature without causing vascular trauma. FIG. 4 is a longitudinal, cross-sectional view of a portion of the tubular body 102, which can be a multi-layer tube. For example, the tubular body 102 can include a lubricious inner liner layer 103, an outer jacket layer 107, and a reinforcement layer 120 that extends at least in part within a region between the inner liner layer 103 and the outer jacket layer 107. One or more of the layers can vary along the length of the catheter to provide different functional capabilities of the navigation catheter 100. For example, the outer jacket layer 107 can incorporate discreet sections of polymer with different durometers, compositions, and/or thicknesses to aid in varying the flexibility along the length of the navigation catheter 100. The reinforcement layer 120 can terminate near where the proximal portion 105 transitions to the distal portion 110. The inner liner layer 103 separates the reinforcement layer 120 from the inner lumen 123 in the proximal portion 105. The outer jacket layer 107 overlays the reinforcement layer 120 in the proximal portion 105 and the inner liner layer 103 in the distal portion 110.

The proximal portion 105 is generally less flexible than the distal portion 110. The reinforcement layer 120 within the proximal portion 105 of the tubular body 102, which can be a braid reinforcement, is designed specifically for pushability and torqueability. The proximal portion 105 can transition to be even stiffer towards the proximal-most end of the proximal portion 105 for additional pushability and support. The proximal portion 105 can be used for bi-directional movement of the navigation catheter 100 within a vessel and/or movement of the navigation catheter 100 within the catheter 200 and/or for bi-directional movement of the navigation catheter 100 and the catheter 200 together simultaneously. This, together with the proximal support provided by the catheter 200, ensures the system 10 can be advanced within the vessel to distal locations without being delivered through a long guide sheath. As discussed above, the stiffness within the proximal portion 205 of the catheter 200 is sufficient to avoid problems of prolapse of the catheter 200, which can be particularly problematic when advancing a catheter through tortuous anatomy alone through a vessel without a guide sheath present. The proximal portion 105 of the tubular body 102 aids in preventing prolapse of the catheter 200. The distal portion 110 of the tubular body 102 aids in navigating the catheter 200 through the aortic arch into the brachiocephalic vessel off the arch and into the internal carotid artery (ICA).

The proximal portion 105 and the distal portion 110 of the navigation catheter 100 will be described in more detail below.

As mentioned, the proximal portion 105 of the tubular body 102 is reinforced to be relatively stiff for pushability and torqueability of the distal portion 110. In an implementation, the reinforcement layer of the proximal portion 105 incorporates a braid reinforcement 120. The braid reinforcement 120 of the proximal portion 105 provides stiffness to the proximal portion 105 of the navigation catheter 100 so that the proximal portion 105 can be used to advance the catheter 200 through a vessel without any long guide sheath. The braid reinforcement 120 can be long enough to extend from the access location (e.g., transfemoral) to within at least a portion of the internal carotid artery (ICA) while also providing for adjustments, if needed. The braid reinforcement 120 can project from the proximal end of the navigation catheter 100 near the proximal hub or luer end 104 up to a distance that is less than the total working length of the navigation catheter 100. In some implementations (e.g., femoral or radial percutaneous access), the braid reinforcement 120 of the proximal portion 105 can be about 120 cm to about 160 cm, preferably about 140 cm to about 155 cm, most preferably about 150 cm to about 153 cm. The length of the braid reinforcement 120 of the proximal portion 105 is sufficient to navigate the curves of the aortic arch and advance distal to the brachiocephalic take-off. The length of the braid reinforcement 120 preferably avoids advancing substantially into the skull and thus, extends up to a location that is just proximal of the carotid siphon.

The braid reinforcement 120 can be a super elastic material, such as nickel titanium alloy or other suitable stiff material, such as stainless steel. In some implementations, the braid reinforcement 120 can be a flat, 304V stainless steel wire that is 0.001″×0.003″ to about 0.001″×0.005″ and having about 50 to about 70 picks per inch (ppi). As an example, the braid reinforcement 120 can incorporate about 16 strands having a pattern of one wire under two wires, one wire over two wires, etc. The geometry, patterns, and lengths of the braid reinforcement 120 allow for the braid reinforcement 120 to flex in a first direction along a first radius of curvature to curve through the aortic arch and flex in a second direction along a second radius of curvature to curve away from the aortic arch into at least the common carotid artery/brachiocephalic take-off from the femoral artery access site.

Preferably, the distal end region of the braid reinforcement 120 avoids entering the skull at significant lengths and transitions to the unreinforced, distal portion 110 of the navigation catheter 100. The reinforced proximal portion 105 can be about 120 cm to about 160 cm, preferably about 140 cm to about 155 cm, most preferably about 150 cm to about 153 cm. The distal portion 110 can be a short segment extending beyond the proximal portion 105 and is about 0.5 cm to about 10 cm, preferably about 1.5 cm to about 2.5 cm. The distal portion 110 is designed to extend at least partially distal to the distal end 206 of the catheter 200 during navigation and aids in navigating the catheter 200 through the aortic arch into the brachiocephalic vessel off the arch and into the internal carotid artery (ICA). As an example, the distal portion 110 of the navigation catheter 100 can be about 2 cm and the reinforced proximal portion 105 of the navigation catheter 100 can be about 153 cm for a total working length of the navigation catheter 100 that is longer than the catheter 200 it delivers and about 155 cm long. The entry location of the catheter system 10 can be in the femoral artery and the target embolus can be distal to the right common carotid RCC artery, such as within the M1 segment of the middle cerebral artery on the right side. The distal end region of the braid reinforcement 120 can remain within a vessel that is proximal to severely tortuous anatomy such as the carotid siphon, the right common carotid RCC artery, the brachiocephalic trunk BT, the take-off into the brachiocephalic artery from the aortic arch, the aortic arch AA as it transitions from the descending aorta DA. This length of the proximal portion 105 relative to the distal portion 110 avoids inserting the stiffer proximal portion 105, or the material transition between the stiffer proximal portion 105 and the distal portion 110 from inserting substantially within the skull while still navigating the turns of the aortic arch into the brachiocephalic take-off.

The lengths of the portions 105, 110, and the navigation catheter 100 length overall can vary depending on the point of insertion, including femoral, carotid, radial, brachial, ulnar, or subclavian arteries. The lengths of the body 102 described herein can be modified to accommodate different access points. For example, a tubular body 102 of the navigation catheter 100 for entry through the femoral artery near the groin may be longer than a tubular body 102 of a navigation catheter 100 for entry through the subclavian artery. The combination of lengths can be selected to ensure the braid reinforcement 120 of the proximal portion 105 remains substantially outside the skull. In some implementations, the length of the braid reinforcement 120 of the proximal portion 105 traverses the aortic arch to a region distal to the brachiocephalic take-off. In an implementation, the braid reinforcement 120 of the proximal portion 105 terminates within the descending aorta DA proximal to the aortic arch AA.

While the reinforcement layer of the navigation catheter 100 is described herein as being a braid, other configurations and materials of the reinforcement layer are considered herein. The reinforcement layer can be made from metal such as 304 stainless steel, Nitinol, Nitinol braid, helical ribbon, helical wire, cut stainless steel, cobalt chromium alloy, or other metal alloy, or the like, or stiff polymer such as PEEK. The reinforcement layer can be laser-cut or machine-cut so as to be flexible in a distal end region as discussed above. The reinforcement layer can provide a desired combination of strength, flexibility, and resistance to crush.

The reinforcement layer can also vary so that combinations of reinforcement are provided along at least a region of the navigation catheter 100. As an example, a proximal portion 105 of the navigation catheter 100 can include a braid to provide good torqueability and the braid can be overlaid by a coil to provide good kink resistance. As another example, the proximal portion 105 of the navigation catheter 100 can include a hypotube to provide good pushability that incorporates interruptions, cut-outs, or other feature at its distal end region to provide increasing flexibility towards the distal end region of the navigation catheter 100.

The navigation catheter 100 has increasing flexibility towards the distal end with material transitions along its length that are spaced to be misaligned with jacket material, pattern and pitch transitions of the catheter 200 to support the catheter 200 and avoid kinks, angulations or sharp bends in its structure, for example, during navigation of severe angulations such as those having 90°, for example at the aorto-iliac junction, the left subclavian take-off from the aorta, the takeoff of the brachiocephalic (innominate) artery from the ascending aorta. The navigation catheter 100 transitions from being less flexible within the braid-reinforced proximal portion 105 to its junction with the distal portion 110 to being more flexible at the distal-most end. The change in flexibility from proximal to distal end of the navigation catheter 100 can be achieved by any of a variety of methods as described herein. The navigation catheter 100 has a distal bending stiffness that is greater than a distal bending stiffness of the catheter 200 and a bending stiffness in the region along at least a portion of the transition section of the catheter 200 that is stiffer than the bending stiffness of the catheter 200 it supports. Thus, the navigation catheter 100 can be supportive for advancing the catheter 200 without a guide sheath.

The braid reinforcement 120 terminates at a distal end of the proximal portion 105 where it transitions to the distal portion 110 of the navigation catheter 100. The distal end of the braid reinforcement 120 can be at least partially covered by a segment of tubing 124 formed of a heat-shrink material, such as PET (see FIG. 4). Thus, the segment of tubing 124 begins near the junction between the proximal portion 105 having the braid reinforcement 120 and the distal portion 110 without the braid reinforcement 120.

The navigation catheter 100 is preferably shaped and provides steerable guidance for vessel selection in navigating the catheter 200 through the aortic arch and up into the brachiocephalic take-off. The shape of the navigation catheter 100 can vary, including any of a variety of vessel selection tip shapes. The navigation catheter 100 can be shaped similarly to a Berenstein vessel selection tip and have a short-angled tip shape. The navigation catheter 100 can be shaped similarly to a Simmons vessel selection tip and have a longer, dual hooked shape. The navigation catheter 100 can be shaped similarly to a Cobra vessel selection tip and have two large-radius curves and ending in a shorter, smaller-radius curve. Where the navigation catheter 100 is described herein as having a particular configuration, it should be appreciated that other vessel selection tip shapes are considered.

At least a distal end region 122 of the distal portion 110 is arranged at an angle to bend or curve relative to a longitudinal axis of a proximal end region 126 of the distal portion 110. FIG. 4 shows the proximal end region 126 of the distal portion 110 is substantially coaxial with a longitudinal axis of the proximal portion 105. The distal end region 122 of the distal portion 110 is curved so that an angle Θ is formed between an outer surface of the distal end region 122 and a horizontal surface. The angle Θ can vary between about 65 degrees to about 85 degrees, preferably about 70 degrees to about 80 degrees. The proximal end region 126 can have a length that is about 5 mm-10 mm and the distal end region 122 can have a length that is about 10 mm-15 mm such that the length of the distal portion 110 is about 15 mm-25 mm. The distal portion 110 including the straight proximal end region 126 and the angled distal end region 122 can be formed of a segment of PEBAX having a shore hardness of about 55D overlaying the inner liner 103. The material properties of the distal portion 110 allows for the angled distal end region 122 to flex into a straight configuration, such as for advancing through the inner lumen 223 of the catheter 200, and return to its angled configuration, such as upon exit of the distal opening 208 of the catheter 200. The distal end region 122 of the distal portion 110 can incorporate a marker band 111 near the distal opening 208 at a radiused tip.

The inner liner 103 can be constructed, as an example, from a low friction polymer such as PTFE (polytetrafluoroethylene) or FEP (fluorinated ethylene propylene) to provide a smooth inner surface. The lubricious inner liner 103 can have one or more thicknesses along variable sections of flexibility. The PTFE liner can be a tubular liner formed by dip coating or film-casting a removable mandrel, such as a silver-plated copper wire mandrel. Various layers can be applied having different thicknesses. For example, a base layer of etched PTFE can be formed having a thickness of about 0.005″. A second, middle layer can be formed over the base layer that is Tecoflex SG-80A having a thickness of about 0.0004″. A third, top layer can be formed over the middle layer that is Tecoflex SG-93A having a thickness of about 0.0001″ or less. The inner liner 103 can extend an entire working length of the tubular body 102, including the distal portion 110. FIG. 4 shows the inner liner 103 extending to the distal-most end 106 of the tubular body 102 distal to the marker band 111 to the distal opening 108.

The outer jacket layer 107 is formed of materials that provide mechanical integrity to the inner liner layer 103, for example, materials such as PEBAX, thermoplastic polyurethane, polyethylene, nylon, or the like. The outer jacket layer 107 may be composed of discreet sections of polymer with different durometers, composition, and/or thickness to vary the flexibility along the length of the tubular body 102. A lower durometer outer jacket material can be used in the distal portion 110 of the navigation catheter 100 compared to the proximal portion 105 of the catheter 100. The proximal portion 105 of the navigation catheter 100 can incorporate outer jacket material having a durometer, for example, 72D PEBAX. The 72D PEBAX material can transition moving distally within the proximal portion 105 to an outer jacket material having a lower durometer, for example, 55D PEBAX or a combination of 72D and 55D PEBAX. The outer jacket layer 107 can transition to more flexible materials within the distal portion 110 having a hardness of about 55D PEBAX or 45D PEBAX approaching the distal end 106. The distal end 106 can be formed of a material having a hardness of no lower than about 45D PEBAX. For example, a first region of the navigation catheter 100, such as a region of the proximal portion 105 incorporating the braid reinforcement 120, can be formed of a material having a hardness of at least about 72D or greater along a first length, a second region of the navigation catheter 100, such as a region of the proximal portion 105 incorporating the braid reinforcement 120, can be formed of a material having a hardness that is less than about 72D, such as about 55D along a second length, a third region of the navigation catheter 100, such as a region of the distal portion 110 excluding the braid reinforcement 120, can be formed of a material having a hardness that is about 55D, along a third length.

The lengths of each of the segments of outer jacket 107 can vary. In some implementations, the distal portion 110 is formed of a single segment of outer jacket layer 103 having a single hardness of about 55D PEBAX that extends from a location near the end of the braid reinforcement 120 to the distal-most end 106 where, for example, a guidewire extends through the distal opening 108. In some implementations, such as the proximal segment can extend along a length of about 150 cm. The regions of the catheter having a hardness of about 72D or 55D can have any of a variety of lengths between about 20 mm, 40 mm, 60 mm, and about 80 mm. The wall thickness of the outer jacket material can be uniform along the length of the tubular body 102 or may be reduced moving distally along the navigation catheter 100.

The material hardnesses described herein with respect to the navigation catheter 100 can be achieved by a single polymer material or by mixtures of polymer materials. For example, a mixture of 55D PEBAX and 72D PEBAX can provide a harder polymeric material than that of 55D PEBAX alone and a softer polymer material than that of 72D PEBAX alone. The polymer segments of the various catheter components described herein can incorporate any of a variety of hardnesses between the specific hardnesses identified by blending of one or more polymer materials to achieve a transition in flexibility along a length of the structure. Additionally, the ranges of hardnesses between the proximal end portions of the catheters described herein and a distal end portions of the catheters can vary from greater than 72D PEBAX (e.g. 72D PEBAX reinforced with a metallic or non-metallic element) down to less than 55D (e.g., 35D PEBAX).

The changes in material hardness of the outer jacket 107, in combination with the changes in the reinforcement from the proximal portion 105 to the unreinforced distal portion 110, may be varied to increase the flexibility moving distally along the navigation catheter 100. As discussed above, the proximal portion 105 of the navigation catheter 100 incorporates the braid reinforcement 120. The ppi of the braid reinforcement 120 may decrease at the distal end region compared to more proximal areas of the reinforcement 120. The ppi and/or pattern in the braid 120 of the proximal portion 105 may be varied to be more flexible near the distal end where the proximal portion 105 transitions to the distal portion 110. The structure of the reinforcement layer (i.e., braid-plus-liner to inner liner only) and/or the materials of the reinforcement layer may change over the length of the tubular body 102.

The navigation catheter 100 can be received within and extended through the internal lumen 223 of the catheter 200 (see FIG. 1C and also FIG. 6A). As mentioned above, the catheter 200 preferably has a large inner diameter to maximize the aspiration force delivered to the site of the occlusion and/or provide ample clearance for delivery of a working device. A suitable size for the inner diameter of the catheter body 202 (i.e., inner diameter of lumen 223) is between 0.054″ and 0.100″, or more preferably between 0.070″ and 0.095″, most preferably between 0.088″ and 0.090″, depending on the patient anatomy and the clot size and composition. The outer diameter of the tubular body 102 of the catheter 100 is selected to correspond substantially to the inner diameter of the catheter body 202. The outer diameter of at least a region of the distal portion 110 of the navigation catheter 100 can be sized to substantially fill at least a portion of the internal lumen 223 of the catheter 200. The tubular body 102 of the distal portion 110 can include an outer diameter near a distal end region of the navigation catheter 100 that approaches the inner diameter of the catheter 200 (i.e., a maximum outer diameter) to minimize a distal ledge effect of the catheter 200 as it traverses the vasculature. A suitable size for the maximum outer diameter of the tubular body 102 to minimize the ledge effect is between 0.042″ and 0.097″, or more preferably between 0.058″ and 0.092″, most preferably between 0.076″ and 0.087″, depending on the size of the catheter 200 being advanced. A difference between the inner diameter of the catheter 200 and the maximum outer diameter of the tubular body 102 to minimize the ledge effect can be no more than about 0.012″, no more than about 0.010″, for example, from 0.003″ up to about 0.012″, about 0.005″ to about 0.010″, preferably about 0.006″ to about 0.008″.

In some implementations, the inner diameter of the distal portion 210 of the catheter 200 can be about 0.070″ to about 0.072″ and the outer diameter of the tubular body 102 can be about 0.062″ to about 0.070″ such that the difference between them is about 0.002″ to about 0.008″ up to about 0.010″. In some implementations, the inner diameter of the distal portion 210 of the catheter 200 can be 0.088″ to about 0.089″ and the outer diameter of the tubular body 102 can be 0.080″ to about 0.082 such that the difference between them is about 0.008″ to about 0.009″. Larger catheter sizes are considered as well (e.g., inner diameters of 0.092″ or 0.105″). The outer diameter of the tubular body 102 can be about 0.005″ to about 0.010″ less than the inner diameter of the catheter being advanced.

The outer diameter of the tubular body 102 can be substantially similar along most of its working length so that the outer diameter of the tubular body 102 within the proximal portion 105 is the same or similar to the outer diameter of the tubular body 102 within the distal portion 110. In other implementations, the outer diameter of the tubular body 102 can gradually decrease moving distally along the tubular body 102 to be smaller at the distal end of the catheter 100. For example, the tubular body 102 can have a relatively uniform outer diameter from a proximal end region to a distal end region up to a point where the braid reinforcement 120 terminates and the distal portion 120 begins and change outer diameter to be smaller toward the distal end 106 of the navigation catheter 100. In still further implementations, the outer diameter of the tubular body 102 can gradually decrease moving proximally along the tubular body 102 to be smaller at the proximal end of the catheter 100 or can incorporate a step-down or taper in outer diameter at a point along a length moving in a proximal direction.

The distal portion 110 located distal to the maximum outer diameter of the proximal portion 105 of the tubular body 102 can be advanced through the catheter lumen 223 to a position where it extends beyond the distal end 206 of the catheter 200. The overall length of the navigation catheter 100 (e.g., between the proximal end through to the distal-most tip) can vary, but generally is long enough to extend through the catheter 200 so that at least a portion of the distal portion 110 extends beyond the distal end 206 of the catheter 200 and at least a region having the maximum outer diameter remains inside the lumen 223 to minimize the distal catheter edge. In some implementations, the overall length of the navigation catheter 100 is about 145 cm to about 160 cm and has a working length of 150 cm to about 155 cm.

Again with respect to FIGS. 2 and 4, the tubular body 102 can have an overall shape profile from proximal end to distal end that transitions from a first outer diameter having a first length to an outer diameter having a second length. The first length of this first outer diameter region (i.e., the maximum outer diameter region) can be sized as described elsewhere herein so that it is less than the inner diameter of the catheter being advanced, for example, by about 0.005″ to about 0.010″. The length between the first outer diameter of the proximal portion 105 and the second outer diameter of the distal portion 110 can be about 6 cm. When the navigation catheter 100 is inserted through the catheter 200, the distal portion 105 is configured to extend beyond and protrude out through the distal end 206 of the distal portion 210 of the catheter 200 whereas the more proximal region of the tubular body 102 having larger outer diameter remains within the lumen 223 of the distal portion 210. The distal end of the distal portion 210 of the catheter 200 can be blunt and have no change in the dimension of the outer diameter whereas the distal portion 105 can be smaller in outer dimension and/or change in outer dimension along its length. The outer diameter of the tubular body 102 also approaches the inner diameter of the distal portion 210 such that the step up from the tubular body 102 to the inner diameter of the distal portion 210 of the catheter 200 is minimized. Minimizing this step-up prevents issues with the lip formed by the distal end of the distal portion 210 catching during advancement of the catheter 200 through a vessel, particularly because the catheter 200 is being advanced directly within a vessel as opposed to through a long guide sheath.

The navigation catheter 100 can have an inner diameter that corresponds to the outer diameter of a guidewire conventionally used for initial advancement into the vessel, which can be about 0.034″ to about 0.036″ compared to distal neuro guidewires having smaller outer diameters (e.g., 0.014″, 0.016″, 0.018, or 0.025″). Thus, the navigation catheter 100 may have an inner diameter of about 0.037″ to about 0.040″. As an example, the distal portion 210 of the catheter 200 can have an inner diameter of about 0.089″. A navigation catheter 100 inserted through the distal portion 210 can have an outer diameter of about 0.080″ to about 0.082″ substantially filling the inner diameter of the distal portion 210. The navigation catheter 100 can have an inner diameter of about 0.037″ to about 0.040″ such that the wall thickness in this region can be about 0.040″ to about 0.045″.

At least a portion of the tubular body 102 of the navigation catheter 100 can have an inner diameter that is substantially constant or uniform and does not change over its length even where the outer diameter may change along the distal end region 322. Thus, the inner diameter of the lumen 123 extending through the tubular body 102 of the navigation catheter 100 can remain uniform and the wall thickness of the distal portion 110 can decrease to provide the change in outer diameter. The wall thickness can thin distally along the length of the distal portion 110.

The materials used to form the regions of the tubular body 102 can include PEBAX elastomers in the Shore D hardness ranges (such as PEBAX 45D, 55D, 63D, 70D, 72D) with or without a lubricious additive compound, such as Mobilize (Compounding Solutions, Lewiston, Maine). The proximal portion 105 can incorporate a lubricious liner layer 103, as discussed elsewhere herein. The liner layer 103 can extend into the distal portion 110 of the tubular body 102.

The gap in ID/OD between the tubular body 102 and the distal portion 210 of the catheter 200 can be about 0.003″-0.012″, more preferably about 0.005″-0.010″, along a majority of their lengths. For example, the tubular body 102 can have a relatively uniform outer diameter (e.g., about 0.082″) from a proximal end region to a distal end region up to a point where the braid reinforcement 120 terminates and the distal end region 122 begins. Similarly, the distal portion 210 of the catheter 200 can have a relatively uniform inner diameter (e.g., about 0.089″) from a proximal end region to a distal end region. As such, the difference between their respective inner and outer diameters along a majority of their lengths can be within this gap size range of 0.003″ to 0.012″. The distal portion 110 of the tubular body 102 may have a larger gap size relative to the inner diameter of the distal portion 210 than the proximal portion 105 due to a difference in outer diameter between the distal portion (e.g., about 0.070″ versus about 0.082″). During use, however, the distal portion 210 is configured to extend distal to the distal end 206 of the catheter 200 such that the region of the tubular body 102 having an outer diameter sized to substantially match the inner diameter of the distal portion 210 of the catheter 200 is positioned within the lumen 223 of the catheter 200 such that it can minimize the lip at the distal end of the catheter 200. The tubular body 102 need not have a uniform outer diameter along its entire length and can incorporate a proximal taper down to a smaller outer diameter in its proximal end region. Although the gap size in these more proximal regions of the catheter system can be larger than the gap size range discussed above, the proximal taper is intended to remain inside the catheter 200 during use.

The flexibility of the tubular body 102 can increase towards the distal portion 110 such that the distal portion 110 of the tubular body 102 is softer, more flexible, and articulates and bends more easily than more proximal regions (e.g., proximal portion 105). The proximal portion 105 of the tubular body 102 incorporates a braid reinforcement 120 that terminates about 2 cm from the distal-most end of the navigation catheter 100. The lack of the reinforcement in the distal portion 110 provides additional flexibility to this region compared to the reinforced proximal portion 105, as discussed above.

The tubular body 102 of the navigation catheter 100 can be generally tubular along at least a portion of its length such that it has a single lumen 123 extending parallel to a longitudinal axis of the navigation catheter 100 (see FIG. 4). The single lumen 123 of the tubular body 102 is sized to accommodate a guidewire. The guidewire can extend through the single lumen 123 generally concentrically from a proximal opening to a distal opening 108 through which the guidewire can extend. In some implementations, the proximal opening is at the proximal end of the navigation catheter 100 such that the navigation catheter 100 is configured for over-the-wire (OTW) methodologies. In other implementations, the proximal opening is a rapid exchange opening through a wall of the navigation catheter 100 such that the navigation catheter 100 is configured for rapid exchange rather than or in addition to OTW.

Guidewires used for initial percutaneous access of a vessel are typically larger than guidewires used for delivery through the vessels in the head. As such, the ID of the navigation catheter 100 can be larger than the ID of the delivery catheter 300 to mate with different guidewire sizes. The ID of the navigation catheter 100 can be about 0.031″ to about 0.040″ whereas the ID of the delivery catheter 300 can be less than 0.031″. The single lumen 123 of the navigation catheter 100 can be configured to receive a guidewire having an outer diameter from about 0.030″ to about 0.037″. The single lumen 123 of the navigation catheter 100 can have an inner diameter at the distal tip (i.e., the size of the distal opening 108 from the single lumen 123) that is at least about 0.030″ up to about 0.045″, about 0.035″ up to about 0.040″ inner diameter. The tubular body 102 can be about 0.002″ greater, or about 0.003″ greater, or about 0.004″ greater in inner diameter than the outer diameter of the guidewire. In an implementation, the guidewire outer diameter is about 0.037″ and the inner luminal diameter of the tubular body 102 is about 0.040″. The difference in size between the distal opening 108 inner diameter of the tubular body 102 and the outer diameter of the guidewire can be between about 0.002″ up to about 0.010″.

The tubular body 102 of the navigation catheter 100 can have a distal portion 110 that changes in outer diameter moving distally from a first outer diameter of the proximal portion 106 to a second outer diameter. The second outer diameter can be about 65%, or 70%, or 75%, or 80%, or 85%, or 90% of the first outer diameter and any of a variety of percentages in between. The braid reinforcement 120 can have a nominal outer diameter at a proximal end region of the proximal portion 105 that is about 0.082″ and a nominal outer diameter at a distal end region is about 0.073″ providing a change in outer diameter over a length of the proximal portion 105 that is about 0.009″. The outer diameter of the distal portion 110 of the navigation catheter 100 over the segment of tubing 124 distal of the braid reinforcement 120 can be about 0.072″ and the outer diameter of the distal portion 110 at the distal marker band 111 can be about 0.070″. Thus, the outer diameter of the navigation catheter 100 from the distal end region of the braid reinforcement 120 changes in outer diameter by about 0.002″. The outer diameter can change from a first outer diameter of about 0.082″ to a second outer diameter of about 0.070″ over a length of about 6 cm. The tubular body 102 of the navigation catheter 100 need not change in outer diameter moving distally. In other words, the distal end 106 of the navigation catheter 100 and the proximal end of the navigation catheter 100 can have substantially the same outer diameter.

The navigation catheter 100 includes one or more radiopaque markers 111. At least one radiopaque marker 111 is disposed near the distal opening 108 at the distal end 106 of the catheter 100. The radiopaque bands 111 may be swaged, painted, embedded, or otherwise disposed in or on the tubular body 102. The marker 111 can be a ring around a circumference of one or more regions of the body 102, including a single ring, or 2 or more rings forming a single marker 111. The marker 111 need not be a ring and can have other shapes or create a variety of patterns that provide orientation to an operator regarding the position of the distal opening 108 within the vessel. The radiopaque marker(s) 111 can be a barium polymer, platinum or platinum-loaded, gold, tantalum, tungsten, tungsten polymer blend, tungsten-loaded or any other substance visible under an x-ray fluoroscope. In some implementations, the radiopaque marker 111 is a platinum-iridium loaded PEBAX.

The proximal end region of the braid reinforcement 120 of the proximal portion 105 of the tubular body 102 can be coupled a proximal hub or luer end 104. The luer end 104 can include a lumen molded into a connector body to connect to the single inner lumen 123 of the navigation catheter 100. The luer end 104 can be constructed of thick-walled polymer tubing or reinforced polymer tubing. The luer end 104 allows for the introduction of devices and the like through the navigation catheter 100. The luer end 104 can be integral to the navigation catheter 100 or the catheter 100 can terminate on a proximal end in a female Luer adaptor to which a separate hemostasis valve component, such as a passive seal valve, a Tuohy-Borst valve or rotating hemostasis valve may be attached.

One or more regions of the navigation catheter 100 can be coated with a lubricious coating, such as a hydrophilic coating on an inner surface and/or an outer surface. Suitable lubricious polymers include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, HYDAK coatings (e.g. B-23K, HydroSleek), and the like, and mixtures and combinations thereof. Hydrophilic polymers may be blended among themselves or with formulated amounts of water insoluble compounds (including some polymers) to yield coatings with suitable lubricity, bonding, and solubility.

### Delivery Catheters

The catheter systems 10 described herein can include a delivery catheter 300 configured to advance the catheter 200 through a patient's vasculature without the aid of a long guide sheath. The catheter 200 has exceptional deliverability for advancement to the level of the M1 or M2 despite the relatively large inner diameter suitable for drawing high aspiration forces. The navigation catheter 100, as discussed above, aids quick and safe advancement of the catheter 200 to reach distal sites, such as site distal to the aortic arch within an internal carotid artery. For example, the navigation catheter 100 can be used to advance the catheter 200 so the distal portion 210 reaches a first location that preferably is distal of the aortic arch and proximal of the carotid siphon CS due to the presence of the metal reinforcement layer that extends up to about 2 cm away from the distal-most end of the catheter 100. The navigation catheter 100 can then be exchanged for the delivery catheter 300, which is specifically designed to further advance the catheter 200 quickly and safely so that the distal portion 210 reaches a second location distal to the first location, such as intracranial vessels distal to a petrous portion of the ICA and beyond without catching on branches within the tortuous anatomy of the skull.

The flexibility and deliverability of the catheter 200 allows the catheter 200 to take the shape of the tortuous anatomy and avoids exerting straightening forces creating new anatomy. The aspiration catheter 200 is capable of this even in the presence of the delivery catheter 300 extending through its lumen 223. Thus, the flexibility and deliverability of the delivery catheter 300 is on par or better than the flexibility and deliverability of the distal portion 210 of the aspiration catheter 200 in that both are configured to reach, for example, the middle cerebral artery (MCA) circulation without straightening out the curves of the anatomy along the way. The catheter 200 and the delivery catheter 300, both individually and assembled as a catheter system 10, are configured to navigate around a 180° bend around a radius as small as 0.050″ to 0.150″ or as small as 0.080″ to 0.120″ without kinking, for example, to navigate easily through the carotid siphon. The catheter 200 and delivery catheter 300 resist kinking and ovalizing even while navigating a tortuous anatomy up to 180°×0.080″ radius bend. The delivery catheter 200 has a distal bending stiffness that is less than a distal bending stiffness of the catheter 200 and that transitions throughout the portion that aligns with the transition section of the catheter 200 thereby aiding to prevent kinking of the system.

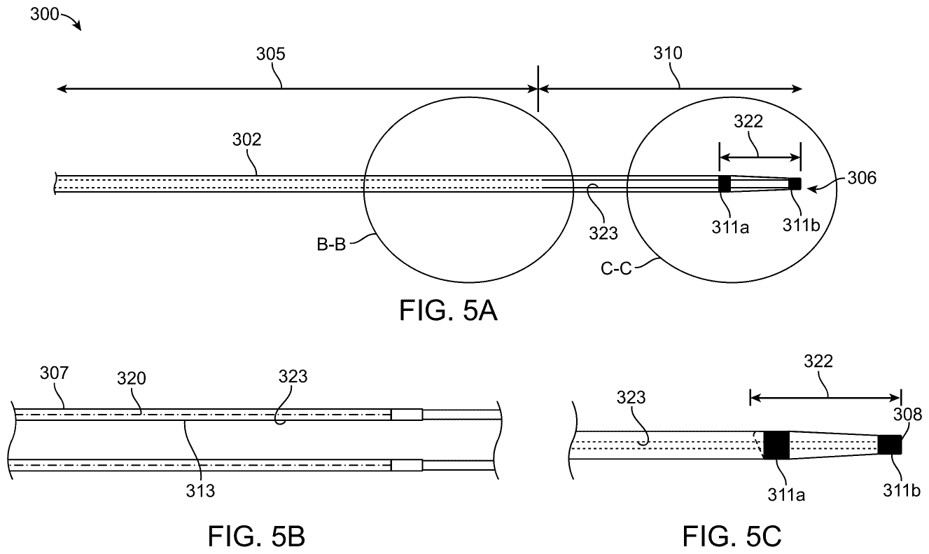

Again with respect to FIG. 2 and also FIGS. 5A-5C, the delivery catheter 300 can include a tubular body 302 defining a single inner lumen 323 having a working length between a proximal end and a distal end of the delivery catheter 300. The tubular body 302 includes a proximal portion 305 that transitions to an exceptionally flexible distal portion 310. The tubular body 302 of the delivery catheter 300 is configured to assume and navigate the bends of the vasculature, particularly the intracranial vasculature. FIG. 5A is a longitudinal, cross-sectional view of the tubular body 302. FIG. 5B is a detailed view taken along line B-B of FIG. 5A illustrating a segment of the proximal portion 305. The proximal portion 305 of the tubular body 302 can be a multi-layer tube including a lubricious inner liner layer 303, an outer jacket layer 307, and a reinforcement layer 320 that lies between the inner liner layer 303 and the outer jacket layer 307. For example, the inner liner layer 303 can separate the reinforcement layer 320 from the inner lumen 323 of the proximal portion 305 and the outer jacket layer 307 overlays the reinforcement layer 320. The reinforcement layer 320 within the proximal portion 305 of the tubular body 302 (e.g., braid-reinforced shaft) is designed specifically for pushability and torqueability to intracranial vessels. FIG. 5C is a detailed view taken at line C-C of FIG. 5A illustrating a segment of the distal portion 510. The distal portion 310 is preferably a single-layer tube without any liner layer or without any reinforcement layer. The unreinforced distal portion 510 is designed specifically for exceptional flexibility to navigate turns of the intracranial vessels.

The proximal portion 305 is generally less flexible than the distal portion 310 and can transition to be even more stiff towards the proximal-most end of the proximal portion 305 for pushability and support. This, together with the proximal support provided by the catheter 200 results in the system 10 needing no long guide sheath within the vessel for delivery. The stiffness of the proximal portion 305 together with the stiffness within the proximal portion 205 of the catheter 200 prevents prolapse of the catheter 200, which can be problematic when a catheter is being advanced through tortuous anatomy alone through a vessel without a long guide present. The distal portion 310 of the delivery catheter 300 is extremely soft and flexible and transitions proximally to the less flexible proximal portion 305. The transition in flexibility of the delivery catheter 300 and the system as a whole is described in more detail below. The proximal portion 305 provides a less flexible proximal end suitable for manipulating the exceptionally flexible, unreinforced, distal portion 310 of the delivery catheter 300. For example, the proximal portion 305 can be used for bi-directional movement of the delivery catheter 300 within a vessel and/or within the catheter 200 and/or for bi-directional movement of the catheter system 10 as a whole.

As mentioned, the proximal portion 305 of the tubular body 302 is reinforced to be suitable for pushability. In an implementation, the reinforcement layer 320 of the proximal portion 305 can be a braid (see FIGS. 5A-5C). The braid reinforcement 320 of the proximal portion 305 provides stiffness to the proximal portion 305 so that the proximal portion 305 can be used to advance the catheter 300 relative to the catheter 200 and through the vessel, particularly through tortuous anatomy of the intracranial vessels, without any long guide sheath. The proximal portion 305 reinforced by the braid reinforcement 320 can be long enough to extend from the access location (e.g., transfemoral) to within at least a portion of the internal carotid artery (ICA) while also providing for adjustments, if needed. The braid reinforcement 320 can project from a luer end 304 at the proximal end of the delivery catheter 300 up to a distance that is less than the total working length of the delivery catheter 300. In some implementations (e.g., femoral or radial percutaneous access), the reinforced proximal portion 305 can be about 90 cm to about 150 cm, preferably about 100 cm to about 140 cm, most preferably about 110 cm to about 130 cm. The length of the braid reinforcement 320 of the proximal portion 305 is sufficient to navigate the curves of the aortic arch and advance distal to the brachiocephalic take-off. The length of the braid reinforcement 320 preferably avoids advancing substantially into the skull and thus, extends up to a location that is just proximal of the carotid siphon.

The braid reinforcement 320 can be a super elastic material, such as nickel titanium allow or other suitable stiff material, such as stainless steel. In some implementations, the braid reinforcement 320 can be a 304V stainless steel wire that is 0.001″×0.003″ to about 0.001″×0.005″ and having about 50 to about 70 picks per inch (ppi). As an example, the braid reinforcement 320 can incorporate 16 strands having a pattern of one wire under two wires, one wire over two wires, etc. The geometry, patterns, and lengths of the braid reinforcement 320 allow for the braid reinforcement 320 to bend in a first direction along a first radius of curvature to curve through the aortic arch and bend in a second direction along a second radius of curvature to curve away from the aortic arch into at least the common carotid artery/brachiocephalic take-off from the femoral artery access site.

Preferably, the distal end region of the braid reinforcement 320 avoids entering the skull at significant lengths and transitions to the unreinforced, distal portion 310 of the delivery catheter 300. The reinforced proximal portion 305 can be about 110 cm to about 130 cm long, preferably about 120 cm. The unreinforced distal portion 310 can be about 25 cm to about 35 cm long, or about 28 cm to about 30 cm. The distal portion 310 is designed to extend at least partially distal to the distal end 206 of the catheter 200 during navigation. As an example, the unreinforced distal portion 310 of the delivery catheter 300 can be about 30 cm and the reinforced proximal portion 305 can be about 125 for a total working length of the delivery catheter 300 that is longer than the catheter 200 it delivers and about 155 cm long. The entry location of the catheter system 10 can be in the femoral artery and the target embolus can be distal to the right common carotid RCC artery, such as within the M1 segment of the middle cerebral artery on the right side. The distal end region of the braid reinforcement 320 can remain within a vessel that is proximal to severely tortuous anatomy such as the carotid siphon, the right common carotid RCC artery, the brachiocephalic trunk BT, the take-off into the brachiocephalic artery from the aortic arch, the aortic arch AA as it transitions from the descending aorta DA. This length of the proximal portion 305 relative to the distal portion 310 avoids inserting the stiffer proximal portion 305, or the material transition between the stiffer proximal portion 305 and the distal portion 310 from inserting substantially within the skull while still navigating the turns of the aortic arch into the brachiocephalic take-off.

The lengths of the portions 305, 310, and the delivery catheter 300 length overall can vary depending on the point of insertion, including femoral, carotid, radial, brachial, ulnar, or subclavian arteries. The lengths of the body 302 described herein can be modified to accommodate different access points. For example, a tubular body 302 of the delivery catheter 300 for entry through the femoral artery near the groin may be longer than a tubular body 302 of a delivery catheter 300 for entry through the subclavian artery. The combination of lengths can be selected to ensure the braid reinforcement 320 of the proximal portion 305 remains substantially outside the skull. In some implementations, the length of the braid reinforcement 320 of the proximal portion 305 traverses the aortic arch to a region distal to the brachiocephalic take-off. In an implementation, the braid reinforcement 320 of the proximal portion 305 terminates within the descending aorta DA proximal to the aortic arch AA.

While the reinforcement layer is described herein as being a braid, however, the configuration and material of the reinforcement layer can vary. The reinforcement layer can be made from metal such as 304 stainless steel, Nitinol, Nitinol braid, helical ribbon, helical wire, cut stainless steel, cobalt chromium alloy, or other metal alloy, or the like, or stiff polymer such as PEEK. The reinforcement layer can be laser-cut or machine-cut so as to be flexible in a distal end region as discussed above. The reinforcement layer can provide a desired combination of strength, flexibility, and resistance to crush.

The reinforcement layer can also vary so that combinations of reinforcement are provided along at least a region of the delivery catheter 300. As an example, a proximal portion 305 of the delivery catheter 300 can include a braid to provide good torqueability and the braid can be overlaid by a coil to provide good kink resistance. As another example, the proximal portion 305 of the delivery catheter 300 can include a hypotube to provide good pushability that incorporates interruptions, cut-outs, or other feature at its distal end region to provide increasing flexibility towards the distal end region of the delivery catheter 300.

The braid reinforcement 320 terminates at a distal end region of the proximal portion 305 where it transitions to the distal portion 310 of the delivery catheter 300. The distal end region of the proximal portion 305 distal of the braid reinforcement 320 engages the proximal end region of the distal portion 310 (see FIGS. 5B-5C). The distal portion 310 is preferably a single-layer tube without any reinforcement and without any inner liner. The carotid siphon CS is an S-shaped part of the terminal ICA beginning at the posterior bend of the cavernous ICA and ending at the ICA bifurcation into the anterior cerebral artery ACA and middle cerebral artery MCA (see FIG. 1A). The distal portion 310 of the delivery catheter 300 can have a length measured from the transition between the braid reinforcement layer 320 to the distal end 306 of the tubular body 302 that is long enough to extend from a region of the internal carotid artery (ICA) that is proximal to the carotid siphon CS to a region of the ICA that is distal to the carotid siphon (CS), including at least the M1 region of the brain.

The length of the unreinforced, distal portion 305 that is capable of being delivered through the intracranial vessels can be about 20 cm, about 25 cm, about 30 cm, about 35 cm, up to about 40 cm. The length of the proximal portion 310 with the braid reinforcement 320 that is advanced distal to the aortic arch and proximal to the carotid siphon can be about 90 cm, about 95 cm, about 100 cm, about 105 cm, about 110 cm, about 115 cm, about 120 cm, or about 125 cm. As an example, the unreinforced distal portion 310 of the delivery catheter 300 can be about 30 cm and the reinforced proximal portion 305 of the delivery catheter 300 can be about 120 cm for a total working length of the delivery catheter 300 that is longer than the catheter 200 it delivers and about 150 cm. For example, the entry location of the catheter system 10 can be in the femoral artery and the target embolus can be distal to the right common carotid RCC artery, such as within the M1 segment of the middle cerebral artery on the right side. The distal end region of the braid reinforcement layer 320 can remain within a vessel that is proximal to severely tortuous anatomy such as the carotid siphon, the right common carotid RCC artery, the brachiocephalic trunk BT, the take-off into the brachiocephalic artery from the aortic arch, the aortic arch AA as it transitions from the descending aorta DA. This length of the proximal portion 305 relative to the distal portion 310 avoids inserting the stiffer proximal portion 305, or the material transition between the stiffer proximal portion 305 and the distal portion 310, from inserting substantially within the skull while still navigating the turns of the aortic arch into the brachiocephalic take-off.

The lengths of the portions 305, 310, and the catheter 300 length overall can vary depending on the point of insertion for the catheter 300, including femoral, carotid, radial, brachial, ulnar, or subclavian arteries. The lengths of the tubular body 302 described herein can be modified to accommodate different access points. For example, a tubular body 302 of a catheter 300 for entry through the femoral artery near the groin may be longer than a tubular body 302 of a catheter 300 for entry through the subclavian artery.

The combination of lengths can be selected to ensure the reinforcement layer 320 the proximal portion 305 remains outside the skull and the unreinforced distal portion 310 navigates the intracranial vessels. In some implementations, the length of the distal portion 310 is sufficient to reach a region of the M1 segment of the middle cerebral artery (MCA) and other major vessels from a region of the internal carotid artery while the reinforcement layer 320 of the proximal portion 305 of the catheter 300 is located proximal to certain tortuous anatomies (e.g. within the descending aorta DA). In an implementation, the distal portion 310 of the catheter 300 has a length sufficient to position its distal end 306 within the M1 segment of the MCA, the reinforcement layer 320 of the proximal portion 305 traverses the aortic arch to a region distal to the brachiocephalic take-off. In an implementation, the distal portion 310 of the catheter 300 has a length sufficient to position its distal end 306 within the M1 segment of the MCA, the reinforcement layer 320 of the proximal portion 305 terminates within the descending aorta DA proximal to the aortic arch AA. The unreinforced distal portion 310 can extend about 30 cm and can enter the intracranial vessels (e.g., carotid siphon and beyond) and the reinforced proximal portion 315 can extend a length of about 120 cm and remain proximal to the carotid siphon.

The flexibility of the tubular body 302 can vary over its length, with increasing flexibility towards the distal portion 310 of the body 302. The distal portion 310 of the catheter 300 is constructed to be flexible and lubricious to safely navigate to the target location. The distal portion 310 is kink resistant and has increasing flexibility towards the distal end 306 with smooth material transitions along its length to prevent any kinks, angulations or sharp bends in its structure, for example, during navigation of severe angulations, such as those having 90° or greater to 180° turns, for example at the aorto-iliac junction, the left subclavian take-off from the aorta, the takeoff of the brachiocephalic (innominate) artery from the ascending aorta and many other peripheral locations just as in the carotid siphon. The distal portion 310 transitions from being less flexible near the junction with the proximal portion 305 to being more flexible at the distal-most end. The change in flexibility from proximal to distal end of the distal portion 310 can be achieved by any of a variety of methods as described herein.

Each of the distal portion 210 of the catheter 200 and the tubular body 302 of the delivery catheter 300 are capable of bending up to about 180 degrees without kinking or ovalizing such that they can be folded over onto themselves forming an inner and an outer radius of curvature. Additionally, the combined system of the tubular body 302 extending through the lumen of the catheter 200 maintains this high degree of flexibility when the components are assembled into a coaxial system. The two components as a system can be folded and maintain similar flexibility as each component individually. The radius of curvature of the folded system is comparable to the radius of curvature of the components individually. The flexibility of the two components when assembled together as a system allows for the system to be folded over on top of itself such that a width across the catheter bodies is less than a minimum width without kinking or ovalizing.

The inner liner 303 of the proximal portion 305 can be constructed, as an example, from a low friction polymer such as PTFE (polytetrafluoroethylene) or FEP (fluorinated ethylene propylene) to provide a smooth inner surface. The lubricious inner liner 303 can have one or more thicknesses along variable sections of flexibility. The PTFE liner can be a tubular liner formed by dip coating or film-casting a removable mandrel, such as a silver-plated copper wire mandrel. Various layers can be applied having different thicknesses. For example, a base layer of etched PTFE can be formed having a thickness of about 0.005″. A second, middle layer can be formed over the base layer that is Tecoflex SG-80A having a thickness of about 0.0004″. A third, top layer can be formed over the middle layer that is Tecoflex SG-93A having a thickness of about 0.0001″ or less. The inner liner 303 preferably extends only through the proximal portion 305 and does not extend through the distal portion 310.

The outer jacket layer 307 is formed of materials that provide mechanical integrity to the catheter 300, for example, materials such as PEBAX, thermoplastic polyurethane, polyethylene, nylon, or the like. The outer jacket layer 307 may be composed of discreet sections of polymer with different durometers, composition, and/or thickness to vary the flexibility along the length of the catheter body 302. A lower durometer outer jacket material(s) can be used in the distal portion 310 of the catheter 300 compared to the proximal portion 305 of the catheter 300. The proximal portion 305 of the catheter 300 can incorporate outer jacket material such as Nylon or 72D PEBAX. The 72D PEBAX material can transition moving distally within the proximal portion 305 to an outer jacket material having a lower durometer, for example, 55D PEBAX or a combination of 72D and 55D PEBAX. The outer jacket layer 307 can transition to increasingly more flexible materials having a hardness of 55D PEBAX, 45D PEBAX, 35D PEBAX, and combinations thereof approaching the distal end 306. The distal end 306 can be formed of a material having a hardness of no more than about 35D PEBAX. For example, a first region of the catheter 300, such as a region of the proximal portion 305, can be formed of a material having a hardness of at least about 72D or greater along a first length, a second region of the catheter 300, such as a region of the proximal portion 305 and/or the distal portion 310, can be formed of a material having a hardness that is less than about 72D, such as about 55D along a second length, a third region of the catheter 300 can be formed of a material having a hardness that is less than about 55D, such as about 35D along a third length. The segments can include combinations of materials, for example, a combination of 55D and 35D. In some implementations, the final distal region of the delivery catheter 300 can be formed of a material that is matched in hardness to a region of the catheter 200, being advanced.

The lengths of each of the segments of outer jacket 307 can vary. In some implementations, such as the segment at a proximal end region of the catheter covering the braid reinforcement layer 320, the segment can extend along a length of about 110 cm-130 cm. The regions of the catheter having a hardness of about 72D, 55D, 55D/35D, 45D, 35D, 25D, 85A, 80A, 65A, 62A and the like, can have any of a variety of lengths. The wall thickness of the outer jacket material can be uniform along the length of the tubular body 302 or may be reduced moving distally along the catheter 300.

The material hardnesses described herein with respect to the delivery catheter 300 can be achieved by a single polymer material or by mixtures of polymer materials. For example, a mixture of 35D PEBAX and 55D PEBAX can provide a harder polymeric material than that of 35D PEBAX alone and a softer polymer material than that of 55D PEBAX alone. The polymer segments of the various catheter components described herein can incorporate any of a variety of hardnesses between the specific hardnesses identified by blending of one or more polymer materials to achieve a transition in flexibility along a length of the structure. Additionally, the ranges of hardnesses between the proximal end portions of the catheters described herein and a distal end portions of the catheters can vary from greater than 72D PEBAX (e.g. 72D PEBAX reinforced with a metallic or non-metallic element) down to less than 35D (e.g., 62A Tecothane).

The changes in material hardness of the outer jacket 307, in combination with the changes in the reinforcement layer 320 (e.g., reinforced proximal portion 305 versus unreinforced distal portion 310), may be varied to increase the flexibility moving distally along the catheter 300. As discussed above, the proximal portion 305 of the delivery catheter 300 incorporates the braid reinforcement 320. The ppi of the braid reinforcement 320 may decrease at the distal end region compared to more proximal areas of the reinforcement 320. The ppi and/or pattern in the braid 320 of the proximal portion 305 may be varied to be more flexible near the distal end where the proximal portion 305 transitions to the distal portion 310. The structure of the reinforcement layer (i.e., braid-plus-liner to unreinforced region) and/or the materials of the reinforcement layer may change over the length of the tubular body 302.

The delivery catheter 300 can be received within and extended through the internal lumen 223 of the catheter 200 (see FIG. 1D and also FIG. 6B). As mentioned above, the catheter 200 preferably has a large inner diameter to maximize the aspiration force delivered to the site of the occlusion and/or provide ample clearance for delivery of a working device. A suitable size for the inner diameter of the catheter body 202 (i.e., inner diameter of lumen 223) is between 0.054″ and 0.100″, or more preferably between 0.070″ and 0.095″, most preferably between 0.088″ and 0.090″, depending on the patient anatomy and the clot size and composition. The outer diameter of the tubular body 302 of the catheter 300 is selected to correspond substantially to the inner diameter of the catheter body 202. The outer diameter of at least a region of the distal portion 310 of the delivery catheter 300 can be sized to substantially fill at least a portion of the internal lumen 223 of the catheter 200. The tubular body 302 of the distal portion 310 can include an outer diameter that provides at least one snug point near a distal end region of the catheter 300 to minimize a distal ledge effect of the catheter 200 as it traverses tortuous anatomy. A suitable size for the outer diameter of the tubular body 302 at the snug point is between 0.042″ and 0.097″, or more preferably between 0.058″ and 0.092″, most preferably between 0.076″ and 0.087″, depending on the size of the catheter 200 being advanced. A difference between the inner diameter of the catheter 200 and the outer diameter of the tubular body 302 at the snug point can be no more than about 0.012″, no more than about 0.010″, for example, from 0.003″ up to about 0.012″, preferably about 0.006″ to about 0.008″.

In some implementations, the inner diameter of the distal portion 210 can be at least about 0.052″ to about 0.054″ and the maximum outer diameter of the tubular body 302 can be about 0.048″ such that the difference between them is about 0.004″ to about 0.006″. In some implementations, the inner diameter of the distal portion 210 can be about 0.070″ to about 0.072″ and the outer diameter of the tubular body 302 can be about 0.062″ to about 0.070″ such that the difference between them is about 0.002″ to about 0.008″ up to about 0.010″. In some implementations, the inner diameter of the distal portion 210 can be 0.088″ to about 0.089″ and the outer diameter of the tubular body 302 can be 0.080″ to about 0.082 such that the difference between them is about 0.008″ to about 0.009″. Despite the outer diameter of the tubular body 302 extending through the lumen of the distal portion 210, the distal portion 210 and the tubular body 302 extending through it in co-axial fashion are flexible enough to navigate the tortuous anatomy leading to the level of M1 or M2 arteries without kinking and without damaging the vessel.

The outer diameter of the tubular body 302 can be substantially similar along most of its working length so that the outer diameter of the tubular body 302 within the proximal portion 305 is the same or similar to the outer diameter of the tubular body 302 within the distal portion 310. In other implementations, the outer diameter of the tubular body 302 can gradually decrease moving proximally along the tubular body 302 to be smaller at the proximal end of the catheter 300 or can incorporate a step-down or taper in outer diameter at a point along a length moving in a proximal direction.

The delivery catheter 300 can also include a distal end region 322 located distal to the at least one snug point of the tubular body 302. The distal end region 322 can have a length and tapers along at least a portion of the length. The distal end region 322 of the delivery catheter 300 can be advanced through the catheter lumen 223 to a position where it extends beyond the distal end 206 of the catheter 200. The overall length of the delivery catheter 300 (e.g., between the proximal end through to the distal-most tip) can vary, but generally is long enough to extend through the catheter 200 so that at least a portion of the distal end region 322 extends beyond the distal end 206 of the catheter 200 and at least the snug point remains inside the lumen 223 to minimize the distal catheter edge catching on branching vessels of the tortuous anatomy of the intracranial vessels. The length of the delivery catheter 300 allows for the distal end 306 to reach cerebrovascular targets within, for example, the M1 or M2 regions. In some implementations, the overall length of the delivery catheter 300 is about 145 to about 160 cm and has a working length of 140 cm to about 155 cm.

The tubular body 302 can have an overall shape profile from proximal end to distal end that transitions from a first outer diameter having a first length to a tapering outer diameter having a second length. The first length of this first outer diameter region (i.e., the snug-fitting region) can be at least about 5 cm up to about 120 cm, preferably about 10 cm to about 100 cm, more preferably about 20 cm to about 70 cm. The length of the tapering outer diameter of the distal end region 322 can be about 0.5 cm to about 4 cm, or about 1 cm to about 3 cm, or about 2 cm to about 2.5 cm. When the delivery catheter 300 is inserted through the catheter 200, the distal end region 322 is configured to extend beyond and protrude out through the distal end 206 of the distal portion 210 whereas the more proximal region of the tubular body 302 having a uniform outer diameter remains within the lumen 223 of the distal portion 210. The distal end of the distal portion 210 can be blunt and have no change in the dimension of the outer diameter whereas the distal end region 322 can be tapered providing an overall elongated tapered geometry of the catheter system 10. The outer diameter of the tubular body 302 also approaches the inner diameter of the distal portion 210 such that the step up from the tubular body 302 to the inner diameter of the distal portion 210 is minimized. Minimizing this step-up prevents issues with the lip formed by the distal end of the distal portion 210 catching on branches within the tortuous neurovasculature, such as the ophthalmic artery branching off near the carotid siphon, when the distal end region 322 bends and curves along within the vascular anatomy.

The delivery catheters 300 can have a smaller inner diameter to correspond to the outer diameter of guidewires conventionally used in the M1 or M2 regions of the intracranial vessels, which are typically about 0.014″ to about 0.018″. Navigation catheters 100 can have an inner diameter at the distal end region that is larger than the inner diameter of delivery catheters 300 to correspond to the outer diameter of guidewires conventionally used during initial vessel penetration, which are typically about 0.034″ to about 0.036″. Thus, the delivery catheter 300 may have an inner diameter at the distal end region of about 0.019″ where the navigation catheter 100 may have an inner diameter of about 0.037″. As a consequence, the delivery catheters 300 and navigation catheters 100 may also have differences in overall wall thickness. As an example, the distal portion 210 can have an outer diameter of about 0.082″ and an inner diameter of about 0.071″. A delivery catheter 300 inserted through the distal portion 210 can have an outer diameter of about 0.062″ substantially filling the inner diameter of the distal portion 210. The delivery catheter 300 can have an inner diameter of about 0.019″ such that the wall thickness in this region can be about 0.043″. When the delivery catheter 300 is assembled with the distal portion 210 of the catheter and the system folded over on itself (i.e., urged into an 180-degree bend), the maximum width across the system can be less than about 0.20″ or less than about 5 mm without ovalizing of either component forming the assembled system. The outer radius of curvature of the assembled system along the bend can be about 0.10″.

As another example, the distal portion 210 can have an outer diameter of about 0.100″ to about 0.102″ and an inner diameter of about 0.089″. A delivery catheter 300 inserted through the distal portion 210 can have an outer diameter of about 0.080″ substantially filling the inner diameter of the distal portion 210. The delivery catheter 300 can have an inner diameter of about 0.019″ such that the wall thickness in this region can be about 0.061″. When the delivery catheter 300 is assembled with the distal portion 210 of the catheter and the system folded over on itself (i.e., urged into an 180-degree bend), the maximum width across the system can be less than about 0.25″ or less than about 6.4 mm without ovalizing of either component forming the assembled system. The outer radius of curvature of the assembled system along the bend can be about 0.13″.

The dimensions provided herein are approximate and each dimensions may have an engineering tolerance or a permissible limit of variation. Use of the term “about” or “approximately” or “substantially” are intended to provide such permissible tolerance to the dimension being referred to. Where “about” or “approximately” or “substantially” is not used with a particular dimension herein means that that dimension need not be exact.

The tubular body 302 of the delivery catheter 300 can have an inner diameter that is substantially constant or uniform and is designed to avoid changing substantially in size over its length even in the presence of the tapering of the distal end region 322. Thus, the inner diameter of the lumen 323 extending through the tubular body 302 of the delivery catheter 300 can remain uniform and the wall thickness of the distal end region 322 can decrease to provide the taper. The wall thickness can thin distally along the length of the taper. Thus, the material properties in combination with wall thickness, angle, length of the taper can all contribute to the overall maximum flexibility of the distal-most end of the distal end region 322. The delivery catheter 300 undergoes a transition in flexibility from the distal-most end towards the snug point where it achieves an outer diameter that is no more than about 0.012″ different from the inner diameter of the catheter 200.

The distal end region 322 of the tubular body 302 can have a transition in flexibility along its length. The most flexible region of the distal end region 322 can be its distal terminus. Moving along the length of the distal end region 322 from the distal terminus towards a region proximal to the distal terminus, the flexibility can gradually approach the flexibility of the distal end of the distal portion 210. For example, the distal end region 322 can be formed of a material having a hardness of no more than 35D or about 62A and transitions proximally towards increasingly harder materials having a hardness of no more than 55D up to the hardness of the proximal portion 305. As mentioned, the proximal portion 305 can be reinforced with a braid 320. The reinforced polymer of the proximal portion 305 can also include reinforcement structures such as a hypotube, coil, or other reinforcement structure or combination of structures. The reinforcement structures can be metallic or nonmetallic reinforcement, such as a rigid polymer.

The materials used to form the regions of the tubular body 302 can include PEBAX elastomers in the Shore D to Shore A hardness ranges (such as PEBAX 25D, 35D, 40D, 45D, 55D, 63D, 70D, 72D) with or without a lubricious additive compound, such as Mobilize (Compounding Solutions, Lewiston, Maine). In some implementations, the material used to form a region of the tubular body 302 can be an aromatic polyether-based thermoplastic polyurethane (e.g., Tecoflex, Tecothane, Lubrizol) in a Shore A hardness range of 90A, 85A, 75A, 62A, 50A. The proximal portion 305 can incorporate a lubricious liner layer 303, as discussed elsewhere herein. The unreinforced distal portion 310 of the tubular body 302 does not have a liner layer 303 and instead incorporates a lubricious additive directly into the polymer. The inner liner layer 303 can provide additional reinforcement to the proximal portion 305 and lack of the inner liner layer 303 can provide additional flexibility to the distal portion 310. It should also be appreciated that the flexibility of the distal end region 322 can be achieved by a combination of flexible lubricious materials and tapered shapes. For example, the length of the distal end region 322 can be kept shorter than 4 cm, but maintain optimum deliverability due to a change in flexible material from distal-most tip towards a more proximal region a distance away from the distal-most tip.

As mentioned above, the tubular body 302 can be constructed to have variable stiffness between the distal and proximal ends of the tubular body 302. The flexibility of the tubular body 302 is highest at the distal-most terminus of the distal end region 322 and can gradually transition in flexibility to approach the flexibility of the distal end of the distal portion 210, which is typically less flexible than the distal-most terminus of the distal end region 322. Upon inserting the delivery catheter 300 through the catheter 200, the region of the tubular body 302 extending beyond the distal end of the distal portion 210 can be the most flexible and the region of the tubular body 302 configured to be aligned with the distal end of the distal portion 210 during advancement in the vessel can have a substantially identical flexibility as the distal end of the distal portion 210 itself. As such, the flexibility of the distal end of the distal portion 210 and the flexibility of the tubular body 302 just proximal to the extended portion (whether tapered or having no taper) can be substantially the same. This provides a smooth transition in material properties to improve tracking of the catheter system through tortuous anatomy. Further, the more proximal sections of the tubular body 302 can be even less flexible and increasingly stiffer. The change in flexibility of the tubular body 302 can be a function of a material difference, a dimensional change such as through tapering, or a combination of the two. The tubular body 302 has a benefit over a microcatheter in that it can have a relatively large outer diameter that is just 0.003″-0.012″ smaller than the inner diameter of the distal portion 210 of the catheter 200 and still maintain a high degree of flexibility for navigating tortuous anatomy. When the gap between the two components is too tight (e.g., less than about 0.003″), the force needed to slide the delivery catheter 300 relative to the catheter 200 can result in damage to one or both of the components and increases risk to the patient during the procedure. The gap results in too tight of a fit to provide optimum relative sliding. When the gap between the two components is too loose (e.g., greater than about 0.010″-0.012″), the distal end of the catheter 200 forms a lip that is prone to catch on branching vessels during advancement through tortuous neurovasculature, such as around the carotid siphon where the ophthalmic artery branches off.

The gap in ID/OD between the tubular body 302 and the distal portion 210 can be in this size range (e.g., 0.003″-0.012″) along a majority of their lengths. For example, the tubular body 302 can have a relatively uniform outer diameter (e.g., about 0.082″) from a proximal end region to a distal end region up to a point where the taper of the distal end region 322 begins. Similarly, the distal portion 210 of the catheter 200 can have a relatively uniform inner diameter (e.g., about 0.089″) from a proximal end region to a distal end region. As such, the difference between their respective inner and outer diameters along a majority of their lengths can be within this gap size range of 0.003″ to 0.012″. The distal end region 322 of the tubular body 302 that is tapered will have a larger gap size relative to the inner diameter of the distal portion 210. During use, however, this tapered distal end region 322 is configured to extend distal to the distal end of the catheter 200 such that the region of the tubular body 302 having an outer diameter sized to match the inner diameter of the distal portion 210 is positioned within the lumen of the catheter 200 such that it can minimize the lip at the distal end of the catheter 200. The tubular body 302 need not have a uniform outer diameter along its entire length and can incorporate a proximal taper down to a smaller outer diameter in its proximal end region. Although the gap size in these more proximal regions of the catheter system can be larger than the gap size range discussed above, the proximal taper is intended to remain inside the catheter 200 during use.

The flexibility of the tubular body 302 can increase towards the distal end region 322 such that the distal portion 310 of the tubular body 302 is softer, more flexible, and articulates and bends more easily than more proximal regions (e.g., proximal portion 305). The bending stiffness of the distal portion 310, particularly more distal regions of the distal portion 310, is flexible enough to navigate tortuous anatomy such as the carotid siphon without kinking. The distal portion 310 of the tubular body 302 is a fully polymeric structure (except perhaps the presence of one or more radiopaque markers) without any reinforcement structures (i.e., braid, coil, fibers, or liner). The fully polymeric, unreinforced distal portion 310 of the tubular body 302 provides a particularly low bending stiffness range described elsewhere herein that results in the delivery catheter 300 being particularly suitable for navigation through tortuous anatomy (e.g., 180 degrees around a radius as small as 2 mm without kinking). The proximal portion 305 of the tubular body 302 incorporates a reinforcement layer 320 that terminates about 10 cm to about 40 cm or from about 15 cm to about 35 cm or from about 25 cm up to about 30 cm from the distal-most end of the delivery catheter 300.

Where the distal portion 310 of the delivery catheter 300 is described herein as being fully polymeric and having no metallic structure for reinforcement or otherwise, the navigation catheter may still incorporate one or more radiopaque markers to identify particular locations along its length. A fully polymeric delivery catheter 300 or a fully polymeric tubular body 302 of the delivery catheter 300 may additionally include radiopaque contrast material embedded in or coating the polymer including barium sulfate, bismuth compounds, tungsten, platinum/iridium, tantalum, platinum, and other metallic materials that absorb x-rays.

The tubular body 302 of the delivery catheter 300 can be generally tubular along at least a portion of its length such that it has a single lumen 323 extending parallel to a longitudinal axis of the delivery catheter 300 (see FIGS. 5A-5C). The single lumen 323 of the tubular body 302 is sized to accommodate a guidewire. Use of the delivery catheter 300 generally eliminates the need for a guidewire lead. The guidewire can extend through the single lumen 323 generally concentrically from a proximal opening to a distal opening through which the guidewire can extend. In some implementations, the proximal opening is at the proximal end of the delivery catheter 300 such that the delivery catheter 300 is configured for over-the-wire (OTW) methodologies. In other implementations, the proximal opening is a rapid exchange opening through a wall of the delivery catheter 300 such that the delivery catheter 300 is configured for rapid exchange rather than or in addition to OTW.

Guidewires used for initial percutaneous access of a vessel are typically larger than guidewires used for delivery through the vessels in the head. As such, the ID of the initial navigation catheter 100 can be larger than the ID of the delivery catheter 300 to mate with different guidewire sizes. The ID of the initial navigation catheter 100 can be about 0.031″ to about 0.040″ whereas the ID of the delivery catheter 300 can be less than 0.031″. The single lumen 323 of the delivery catheter 300 can be configured to receive a guidewire having an outer diameter from about 0.010″ up to about 0.024″, or in the range of 0.012″ and 0.022″ outer diameter, or in the range of between 0.014″ and 0.020″ outer diameter. The single lumen 323 of the delivery catheter 300 can have an inner diameter at the distal tip (i.e., the size of the distal opening from the single lumen 323) that is at least about 0.010″ up to about 0.030″, about 0.012″ up to about 0.026″ inner diameter, or about 0.016″ up to about 0.024″ inner diameter, or about 0.020″ up to about 0.022″ inner diameter, or about 0.019″ and about 0.021″. The tubular body 302 can be about 0.002″ greater, or about 0.003″ greater, or about 0.004″ greater in inner diameter than the outer diameter of the guidewire. In an implementation, the guidewire outer diameter is between 0.014″ and about 0.022″ and the inner luminal diameter of the tubular body 302 is between 0.020″ and 0.024″. The difference in size between the distal opening inner diameter of the tubular body 302 and the outer diameter of the guidewire can be between about 0.002″ up to about 0.010″.

The inner diameter of the tubular body 302 can be constant along its length even where the single lumen passes through the tapering distal end region 322. Alternatively, the inner diameter of the tubular body 302 can have a first size through the tapering distal end region 322 and a second, larger size through the cylindrical section of the tubular body 302. The cylindrical section of the tubular body 302 can have a constant wall thickness or a wall thickness that varies to a change in inner diameter of the cylindrical section. As an example, the outer diameter of the cylindrical section of the tubular body 302 can be about 0.080″ to about 0.082″. The inner diameter of the tubular body 302 within the cylindrical section can be uniform along the length of the cylindrical section and, for the delivery catheter, can be about 0.019″ to about 0.021″. The wall thickness in this section, in turn, can be about 0.061″. As another example, the outer diameter of the cylindrical section of the tubular body 302 can again be between about 0.080″ to about 0.082″. The inner diameter of the tubular body 302 within the cylindrical section can be non-uniform along the length of the cylindrical section and can step-up from a first inner diameter of about 0.019″ to a larger second inner diameter of about 0.021″. The wall thickness, in turn, can be about 0.061″ at the first inner diameter region and about 0.059″ at the second inner diameter region. The wall thickness of the cylindrical portion of the tubular body 302 can be between about 0.050″ to about 0.065″. The wall thickness of the tapered distal end region 322 near the location of the proximal marker band 311 can be the same as the cylindrical portion (between about 0.050″ and about 0.065″) and become thinner towards the location of the distal marker band. As an example, the inner diameter at the distal opening from the single lumen can be about 0.019″ to about 0.021″ and the outer diameter at the distal opening (i.e. the outer diameter of the distal marker band) and be about 0.030″ to about 0.031″ resulting in a wall thickness of about 0.010″ to about 0.011″ compared to the wall thickness of the cylindrical portion that can be up to about 0.065″. Thus, the outer diameter of the distal end region 322 can taper as can the wall thickness. The wall thickness ranges for the initial navigation catheter 300 can be similarly structured as the delivery catheter 300 as discussed above. The initial navigation catheter 300 may generally have a larger inner diameter to accommodate the larger guidewire, as discussed elsewhere herein, and thus, wall thickness ranges would be adjusted accordingly.

The tubular body 302 of the delivery catheter 300 can have a distal end region 322 with a tapered portion that tapers distally from a first outer diameter to a second outer diameter. The second outer diameter can be about 30%, 35%, 40%, 45%, 50%, 55%, 60%, up to about 65% of the first outer diameter and any of a variety of percentages in between.

The tubular body 302 can have one or more radiopaque markers 311 along its length. The one or more markers 311 can vary in size, shape, and location. One or more markers 311 can be incorporated along one or more parts of the delivery catheter 300, such as a tip-to-tip marker, a tip-to-taper marker, an RHV proximity marker, a Fluoro-saver marker, or other markers providing various information regarding the relative position of the delivery catheter 300 and its components. In some implementations, a distal end region can have a first radiopaque marker 311a and a second radiopaque marker 311b can be located to indicate the border between the tapering of the distal end region 322 and the more proximal region of the tubular body 302 having a uniform or maximum outer diameter. This provides a user with information regarding an optimal extension of the distal end region 322 relative to the distal end of the distal portion 210 to minimize the lip at this distal end of the distal portion 210 for advancement through tortuous anatomy. In other implementations, for example where the distal end region 322 is not necessarily tapered, but instead has a change in overall flexibility along its length, the second radiopaque marker 311b can be located to indicate the region where the relative flexibilities of the tubular body 302 (and the distal end of the distal portion 210 are substantially the same. In still further implementations, the distal end region 322 has a single radiopaque marker 311 that identifies the maximum outer diameter just proximal to the tapering region or a single radiopaque marker 311 that identifies the entire taper. An additional marker 311 can be located to identify the transition between the reinforced proximal portion 305 and unreinforced distal portion 310.

The marker material may be a barium sulfate, bismuth, platinum/iridium band, a tungsten, platinum, or tantalum-impregnated polymer, or other radiopaque marker that does not impact the flexibility of the distal end region 322 and tubular body 302. In some implementations, the radiopaque markers are extruded PEBAX loaded with tungsten for radiopacity. In some implementations, the proximal marker band can be a first width, such as about 2.0 mm wide, and the distal marker band can be a second different width, such as about 2.5 mm wide, to provide discernable information about the distal end region 322. Some marker materials may impact the flexibility of the polymer within which they are embedded. For example, barium sulfate tends to stiffen polymer. Thus, the polymer where the marker material is incorporated may have a reduced hardness to achieve a final material property for the region that remains suitable for navigation. The distal end region 322 may incorporate a radiopaque marker that is a band of barium sulfate-loaded PEBAX that has a final durometer of no greater than 35D, or 25D, or another soft durometer. The PEBAX prior to the embedding of the radiopaque material may have an initial durometer that is less than the final durometer. The reduction in polymer hardness can offset the stiffening effects of the marker material so that the device maintains flexibility suitable for navigating tortuous anatomy (e.g., tip flexibility measurement that is less than about 0.05 Newtons and/or a catheter system capable of bending 180 degrees while maintaining a maximum folded width across that is less than about 5.0 mm without kinking or ovalizing).

### Assembly and Methods of Use

The traditional approach to reach the Circle of Willis is to use a triaxial system including a guidewire placed within a conventional microcatheter placed within an intermediate catheter, and the entire coaxial system extend through a base catheter or long guide sheath. The long guide sheath is typically positioned through an introducer sheath and advanced so that the distal tip of the long guide sheath is placed in a high cervical carotid artery. Coaxial catheter systems advanced through the long guide sheath are often advanced in unison up to about the terminal carotid artery where the conventional coaxial systems must then be advanced in a step-wise fashion in separate throws. This is due to the two sequential 180-degree or greater turns of the brachiocephalic take-off and then the carotid siphon (see FIGS. 1A-1D). A first turn occurs from the descending aorta DA into the brachiocephalic artery. Another turn is at the level of the petrous to the cavernous internal carotid artery. A further turn is at the terminal cavernous carotid artery as it passes through the bony elements and reaches the bifurcation into the anterior cerebral artery ACA and middle cerebral artery MCA. The S-shape region of these last two turns is referred to herein as the “siphon” or “carotid siphon”. The ophthalmic artery arises from the cerebral ICA, which represents a common point of catheter hang up in accessing the anterior circulation.

Conventional microcatheter systems can be advanced through to the anterior circulation over a guidewire. Because the inner diameter of the conventional microcatheter is significantly larger than the outer diameter of the guidewire over which it is advanced, a lip can be formed on a distal end region of the system that can catch on these side branches during passage through the siphon. Thus, conventional microcatheter systems (i.e., guidewire, microcatheter, and intermediate catheter) are conventionally advanced through the bends of the carotid siphon using a step-wise advancement technique one at a time rather than simultaneously in a single smooth pass to distal target sites. For example, to pass through the carotid siphon, the conventional microcatheter is held fixed while the guidewire is advanced alone a first distance (i.e., through the first turn of the siphon). Then, the guidewire is held fixed while the conventional microcatheter is advanced alone through the first turn over the guidewire. Then, the conventional microcatheter and guidewire are held fixed while the intermediate catheter is advanced alone through the first turn over the microcatheter and guidewire. The process repeats in order to pass through the second turn of the siphon, which generally is considered the more challenging turn into the cerebral vessel. The microcatheter and intermediate catheter are held fixed while the guidewire is advanced alone a second distance (i.e., through the second turn of the siphon). Then, the guidewire and interventional catheter are held fixed while the microcatheter is advanced alone through that second turn over the guidewire. Then, the guidewire and the microcatheter are held fixed while the interventional catheter is advanced alone through the second turn. This multi-stage, step-wise procedure is a time-consuming process that requires multiple people performing multiple hand changes on the components. For example, two hands to fix and push the components over each other forcing the user to stage the steps as described above. The step-wise procedure is required because the stepped transitions between these components (e.g., the guidewire, microcatheter, and intermediate catheter) makes advancement too challenging.

In contrast, the catheters 200 described herein are designed to be advanced through vessels directly without the presence of a long guide sheath. The catheter 200 can be advanced initially using the navigation catheter 100 assembled co-axially within the catheter 200 for insertion into a vessel up to the level of about the high cervical carotid artery. The navigation catheter 100 can then be exchanged for the delivery catheter 300 to further advance the catheter 200 through the carotid siphon CS into the intracranial vessels. The catheter 200 and delivery catheter 300 can be advanced as a single unit through both turns of the carotid siphon CS in a single smooth pass or “throw” to a target in an intracranial vessel without the step-wise adjustment of their relative extensions and without relying on the conventional step-wise advancement technique, as described above with conventional microcatheters. The catheter 200 having the navigation catheter 300 extending through it allows a user to advance them in unison in the same relative position from the first bend of the siphon through the second bend beyond the terminal cavernous carotid artery into the ACA and MCA. Importantly, the advancement of the two components can be performed in a single smooth movement through both bends without any change of hand position.

The navigation catheter 100 can be in a juxtapositioned relative to the catheter 200 that provides an optimum relative extension between the two components (see FIG. 6A) for single smooth advancement to a first location. The navigation catheter 100 can be positioned through the lumen 223 of the catheter 200 such that at least the distal end region 122 extends beyond a distal end 206 of the catheter 200. The distal end region 122 of the navigation catheter 100 eliminates the stepped transition between the guidewire and the outer catheter 200 thereby avoiding issues with the distal edge of the catheter 200 catching on tissues. The optimum relative extension, for example, can be the distal end region 122 of the tubular body 102 extending distal to a distal end 206 of the catheter 200 as described elsewhere herein. A length of the distal end region 122 extending distal to the distal end 206 can be about 1.2 cm-2.0 cm. This juxtaposition can be a locked engagement with a mechanical element or simply by a user holding the two components together.

The delivery catheter 100 exchanged for the navigation catheter 100 can be in a juxtapositioned relative to the catheter 200 that provides an optimum relative extension between the two components (see FIG. 6B) for single smooth advancement beyond the first location. The delivery catheter 300 can be positioned through the lumen of the catheter 200 such that its distal end region 322 extends beyond a distal end 206 of the catheter 200. The distal end region 322 of the delivery catheter 300 eliminates the stepped transition between the guidewire and the outer catheter 200 thereby avoiding issues with catching on branching vessels within the region of the vasculature such that the catheter 200 may easily traverse the multiple angulated turns of the carotid siphon CS. The optimum relative extension, for example, can be the distal end region 322 of the tubular body 302 extending distal to a distal end 206 of the catheter 200 as described elsewhere herein. A length of the distal end region 322 extending distal to the distal end can be about 0.5 cm-4.0 cm. This juxtaposition can be a locked engagement with a mechanical element or simply by a user holding the two components together.

The catheter 200 can be advanced together with the navigation catheter 100 or the delivery catheter 300 with a guidewire, over a guidewire pre-positioned, or without any guidewire at all. In some implementations, a first guidewire can be pre-assembled with the navigation catheter 100 and catheter 200 such that the guidewire extends through a lumen of the navigation catheter 100, which is loaded through a lumen of the catheter 200, all prior to insertion into the patient. The pre-assembled components can be simultaneously inserted into and advanced together within a vessel to a first location, such as the cervical portion of the internal carotid artery. A short introducer sheath (e.g., an 8Fr sheath that is about 11-30 cm in length that passes the vessel wall or peel-away sheath) may be used for initial access, but the pre-assembled components need no long guide sheath for support and can be advanced through the vessel alone rather than through another catheter. The lack of the long guide sheath means the arteriotomy can be minimized because the proximal outer dimension of the catheter 200 is smaller than typical long guide sheaths (e.g., 6Fr) without compromising luminal area of the catheter 200. The catheter 200 can be maintained at this first position and the navigation catheter 100 and first guidewire exchanged for a second, smaller guidewire and delivery catheter 300 for advancing past the turns of the carotid siphon. The guidewire can be positioned within a portion of the lumen of the delivery catheter 300, but not extend distal to the distal opening from the lumen so that the distal-most end of the guidewire remains housed within the delivery catheter 300 for optional use in a step of the procedure. For example, the delivery catheter 300 having a guidewire parked within its lumen proximal to the distal opening can be used to deliver the catheter 200 to a target location or near a target location. The guidewire can be advanced distally while the delivery catheter 300 and catheter 200 remain in a fixed position until a distal end of the guidewire is advanced beyond the distal opening a distance. The delivery catheter 300 with or without the catheter 200 can then be advanced over the guidewire that distance. The guidewire can then be withdrawn inside the lumen of the delivery catheter 300.

The catheter 200 can be advanced using the navigation catheter 100 and the delivery catheter 300 interchangeably positioned within the catheter 200. The catheter 200 has sufficient proximal reinforcement provided by a hypotube 215 in its proximal portion 205 that avoids the need for a long guide sheath, which avoids enlarging the arteriotomy into the vessel. The hypotube 215 has increasingly flexible properties towards the distal end, such as by laser-cutting the hypotube 215 thereby creating spirals of metal material that increases its ability to flex and take turns of the aortic arch and enter the brachiocephalic artery. The reinforcement layer transitions to an increasingly flexible distal coil in the distal portion 210 that is capable of being advanced further into the intracranial vessels, including through the carotid siphon, while the hypotube 215 reinforcement remains proximal to the carotid siphon. The coil reinforcement 220 of the distal portion 210 prevents the large inner diameter of the catheter 200, which is well-suited for drawing aspiration through the catheter system 10 and/or large interventional tools to sites within the distal vasculature, from kinking or collapsing. The fully reinforced navigation catheter 100 aids in advancing the catheter 200 to at least the level of the cervical internal carotid artery. The tubular body 102 of the navigation catheter 100 is reinforced along nearly its entire working length (e.g., up to about the last 2 cm of catheter length) with a braid. The braid provides sufficient torqueability and pushability for advancing the catheter 200 to this initial level over a larger guidewire size. The navigation catheter 100 can be exchanged for the delivery catheter 300 for advancing the catheter 200 to distal sites. The proximal portion 305 of the delivery catheter 300 is reinforced, such as with a braid, that provides sufficient pushability to advance the unreinforced distal portion 310 of the delivery catheter 300. The unreinforced distal portion 310 has a length and flexibility sufficient to traverse the carotid siphon and into the M1 and M2 levels of the intracranial vessels. Both the navigation catheter 100 and the delivery catheter 300 have tubular bodies that have outer dimensions designed to substantially correspond to the large inner diameter of the catheter 200 and inner dimensions designed to substantially correspond to the outer diameter of the guidewire with which it is used. For example, the navigation catheter 100 can have a slightly larger inner diameter compared to the inner diameter of the delivery catheter 300 so that it is sized to receive the slightly larger outer diameter of an initial access guidewire (e.g., 0.034″-0.035″) and the delivery catheter 300 has a slightly smaller inner diameter compared to the inner diameter of the navigation catheter 100 so that it is sized to receive the slightly smaller outer diameter of a distal guidewire (e.g., 0.014″-0.019″). In some implementations, the inner diameter of the delivery catheter 300 is uniform and does not change significantly from the proximal end to the distal opening of the delivery catheter 300. In other implementations, the inner diameter of the delivery catheter 300 undergoes a change in size between the proximal end and the distal opening. For example, the proximal portion of the delivery catheter 300 can have at least a first inner diameter and the distal portion of the delivery catheter 300 can have a second inner diameter that is smaller than the first inner diameter. The smaller, second inner diameter can extend along a length to the distal opening (e.g., about 30 cm) and can be less than about 0.024″. The first inner diameter can be greater than 0.024″ up to about 0.045″. The proximal portion with the larger first inner diameter can be sized to receive a larger guidewire (e.g., 0.034″-0.035″) to provide proximal support for the catheter 300, if desired. The distal portion with the smaller second inner diameter can be sized to hug the outer diameter of smaller guidewires for distal sites (e.g., 0.014″-0.019″ guidewires) for navigation purposes. The transition between the inner diameter can be a step-change or a taper. The distal end region 322 of the delivery catheter 300 tapers to a smaller outer diameter at the distal-most end (e.g., 0.031″) compared to the distal portion 110 of the navigation catheter 100, that has an outer diameter (e.g., 0.040″) that is smaller than the proximal portion 105 outer diameter, but not as small as the corresponding outer diameter of the delivery catheter 300. The distal end region 322 of the delivery catheter 300 changes from the first outer diameter to the second outer diameter over a shorter length (e.g., 0.5 cm-4.0 cm) compared to the length of the change in outer diameter that occurs between the proximal portion 105 and distal portion 110 of the navigation catheter 100 (e.g., 6.0 cm-10 cm).

The catheter 200 can be packaged together in a kit with one or both of the navigation catheter 100 and the delivery catheter 300. The catheter and the navigation catheter 100 may be releaseably, pre-packaged in a locked position according to any of a variety of methods (e.g. shrink-wrap, and other known methods). The navigation catheter 100, catheter 200, and delivery catheter 300 may also be packaged separately.

### Materials

One or more components of the catheters described herein may include or be made from a variety of materials including one or more of a metal, metal alloy, polymer, a metal-polymer composite, ceramics, hydrophilic polymers, polyacrylamide, polyethers, polyamides, polyethylenes, polyurethanes, copolymers thereof, polyvinyl chloride (PVC), PEO, PEO-impregnated polyurethanes such as Hydrothane, Tecophilic polyurethane, Tecothane, PEO soft segmented polyurethane blended with Tecoflex, thermoplastic starch, PVP, and combinations thereof, and the like, or other suitable materials.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276R, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material and as described elsewhere herein.

Inner liner materials of the catheters described herein can include low friction polymers such as PTFE (polytetrafluoroethylene) or FEP (fluorinated ethylene propylene), PTFE with polyurethane layer (Tecoflex). Reinforcement layer materials of the catheters described herein can be incorporated to provide mechanical integrity for applying torque and/or to prevent flattening or kinking such as metals including stainless steel, Nitinol, Nitinol braid, helical ribbon, helical wire, cut stainless steel, stainless steel braid, or the like, or stiff polymers such as PEEK. Reinforcement fiber materials of the catheters described herein can include various high tenacity polymers like Kevlar, polyester, meta-para-aramide, PEEK, single fiber, multi-fiber bundles, high tensile strength polymers, metals, or alloys, and the like. Outer jacket materials of the catheters described herein can provide mechanical integrity and can be contracted of a variety of materials such as polyethylene, polyurethane, PEBAX, nylon, Tecothane, and the like. Other coating materials of the catheters described herein include paralene, Teflon, silicone, polyimide-polytetrafluoroethylene, and the like.

### EXAMPLES

The catheters described herein were tested using three-point bend testing to assess bending flexibility along the length of the catheter compared to known catheters. The catheter was gripped at two points 3 cm apart. Each catheter was deflected a distance with an anvil positioned centrally between the two points and force measured in Newtons (N). The test catheter data is an average of two different test catheters.

### Example 1

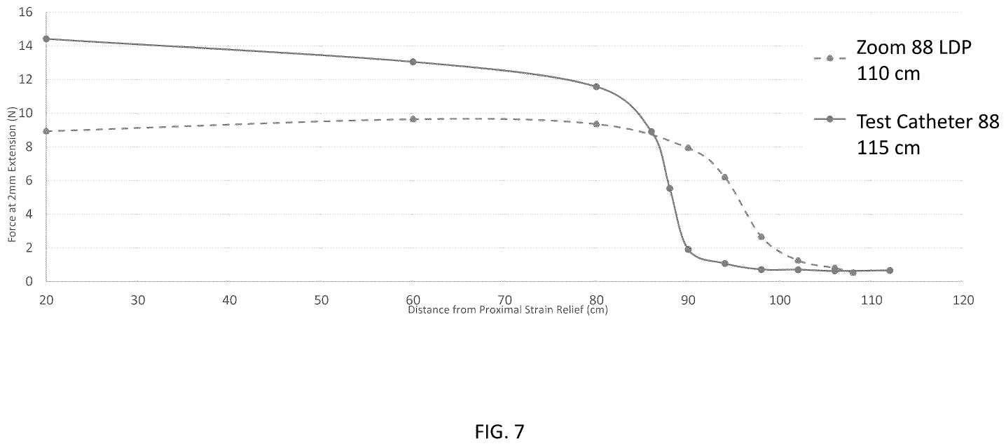

FIG. 7 illustrates the bending flexibility along a length of a test catheter 88 (solid lines) compared to a Zoom 88 LDP catheter 110 cm by Imperative Care, Inc. (Campbell, CA) (hashed lines). The test catheter 88 had a 115 cm working length and an inner diameter of 0.088″. The proximal portion of the test catheter was reinforced with a laser cut stainless steel hypotube having a wall thickness of about 0.004″, a total length of about 80-100 cm, and cuts along 100% of the length. The design of the cuts resembled the embodiment shown in FIG. 3G described above. The distal portion of the test catheter 88 was reinforced by a stainless steel coil having a thickness of about 0.004″, 0.010″ wide, and a pitch of about 0.027″. The test catheter incorporated a PTFE liner and an outer jacket formed of 9 polymer segments of continuously increasing durometers moving distal-to-proximal. The polymer segments along the proximal portion of the test catheter 88 were of a durometer greater than about 55D shore hardness and segments of the distal portion of the test catheter 88 were of a durometer less than or equal to about 25D shore hardness.

FIG. 7 shows the Zoom 88 LDP catheter had a bending flexibility at 20 cm from the proximal strain relief of 8.9 N, a bending flexibility at 90 cm of 7.9 N, and a bending flexibility at 94 cm of about 6.2 N and a distal-most bending flexibility of about 0.52 N at 108 cm from the proximal strain relief. The test catheter 88 had a bending flexibility at 20 cm of about 14.4 N, a bending flexibility at 90 cm of about 1.9 N, and a bending flexibility at 94 cm of about 1.1 N, and a bending flexibility at 106 cm of about 0.63 N.

The test catheter 88 was over 1.6× stiffer at the proximal end compared to the Zoom 88 LDP catheter and the distal end was similarly as flexible as the distal end of the Zoom 88 LDP catheter. The bending flexibility of the test catheter 88, however, remained at least than about 1 N for a length greater than 10 cm. The transition section of the test catheter 88 had a greater change in flexibility (11.6 N to 1.1 N) over a shorter distance (14 cm) compared to the Zoom 88 LDP, which changed from 9.3 N to 1.2 N over a distance of 22 cm. The difference in bending flexibility for the Zoom 88 LDP between the proximal end and the distal end was about 17× compared to a 22× difference in bending flexibility for the test catheter 88 between the proximal end and the distal end.

The test catheter 88 showed a greater change in flexibility over a shorter distance without kinking with the laser cut hypotube, hypotube-to-coil transition configuration, and staggered transitions providing the kink resistance over this transition in flexibility.

### Example 2

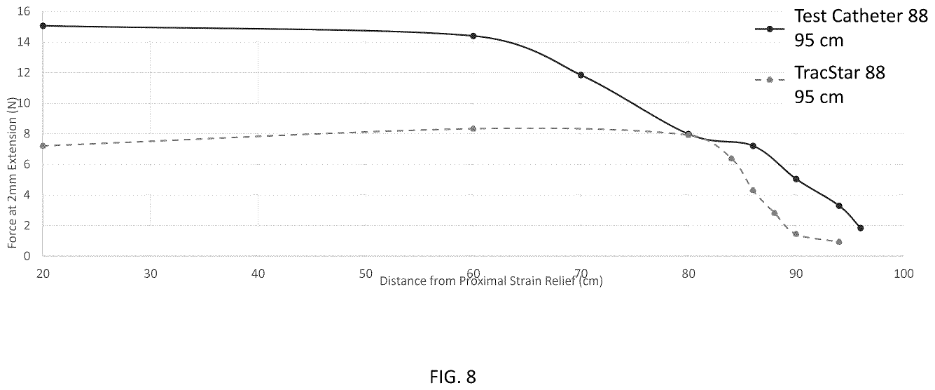

FIG. 8 illustrates the bending flexibility along a length of a test catheter 88 95 cm (solid lines) compared to a TracStar 88 95 cm guide catheter by Imperative Care, Inc. (Campbell, CA). The test catheter 88 had a 95 cm working length and an inner diameter of 0.088″. The proximal portion of the test catheter 88 was reinforced with a laser cut stainless steel hypotube having a wall thickness of about 0.004″, a total length of about 70-90 cm, and cuts along 100% of the length. The design of the cuts resembled the embodiment shown in FIG. 3G described above, adjusted for the overall shorter length of the catheter. The distal portion of the test catheter 88 was reinforced by a stainless steel coil having a thickness of about 0.004″, 0.010″ wide, and a pitch of about 0.027″. The test catheter 88 incorporated a PTFE liner and an outer jacket formed of plurality of polymer segments. The polymer segments along the proximal portion of the test catheter 88 were of a durometer greater than about 55D shore hardness and segments of the distal portion of the test catheter 88 were of a durometer less than or equal to about 25D shore hardness.

FIG. 8 shows the TracStar 88 catheter had a bending flexibility at about cm from the proximal strain relief of about 7.2 N, a bending flexibility at 60 cm of about 8.3 N, a bending flexibility at 86 cm of about 4.3 N, and a bending flexibility at 90 cm of about 1.4 N. The test catheter 88 had a bending flexibility at 20 cm from the proximal strain relief of about 15.1 N, a bending flexibility at 60 cm of about 14.4 N, a bending flexibility at 86 cm of about 7.2 N, and a bending flexibility at 90 cm of about 5 N.

The test catheter 88 was more than about twice as stiff at the proximal end than the TracStar 88 catheter with the same inner diameter and had a distal end that was similar in flexibility to the TracStar 88. The test catheter 88 was a better supportive short catheter. The test catheter 88 provides more proximal support for advancing its distal end region to target occlusions that are located less distal than, for example, the M1. The test catheter 88 also provides more proximal support for advancing other catheters to more distal sites in the neurovasculature.

Implementations describe catheters and delivery systems and methods to deliver catheters to target anatomies. However, while some implementations are described with specific regard to delivering catheters to a target vessel of a neurovascular anatomy such as a cerebral vessel, the implementations are not so limited and certain implementations may also be applicable to other uses. For example, the catheters can be adapted for delivery to different neuroanatomies, such as subclavian, vertebral, carotid vessels as well as to the coronary anatomy or peripheral vascular anatomy, to name only a few possible applications. It should also be appreciated that although the systems described herein are described as being useful for treating a particular condition or pathology, that the condition or pathology being treated may vary and are not intended to be limiting. Use of the terms “embolus,” “embolic,” “emboli,” “thrombus,” “occlusion,” etc. that relate to a target for treatment using the devices described herein are not intended to be limiting. The terms may be used interchangeably and can include, but are not limited to a blood clot, air bubble, small fatty deposit, or other object carried within the bloodstream to a distant site or formed at a location in a vessel. The terms may be used interchangeably herein to refer to something that can cause a partial or full occlusion of blood flow through or within the vessel.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. However, such terms are provided to establish relative frames of reference, and are not intended to limit the use or orientation of the catheters and/or delivery systems to a specific configuration described in the various implementations.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value. One inch or 1″ corresponds to 2.54 cm (SI-units). The recitation of numerical ranges by endpoints includes all numbers within that range (e.g., 1 to 5 includes 1, 1.5, 2, 2.75, 3, 3.80, 4, and 5 and any number in between).

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

The components of the systems disclosed herein may be packaged together in a single package or separately. The finished package would be sterilized using sterilization methods such as Ethylene oxide or radiation and labeled and boxed. Instructions for use may also be provided in-box or through an internet link printed on the label.

### P EMBODIMENTS

P Embodiment 1. A catheter comprising a catheter body defining an inner lumen having a working length between a proximal end and a distal end of the catheter body, wherein the catheter body comprises a proximal portion and a distal portion, wherein the proximal portion is stiffer than the distal portion, wherein the catheter is configured to be advanced through a patient's vasculature so the distal portion reaches an intracranial vessel without prolapse of the proximal portion.
P Embodiment 2. A system comprising: a neurocatheter comprising a catheter body defining an inner lumen and having a working length between a proximal end and a distal end of the catheter body, wherein the catheter body comprises a proximal portion and a distal portion, wherein the proximal portion is stiffer than the distal portion; and a delivery catheter comprising a tubular body defining an inner lumen and having a working length between a proximal end and a distal end of the tubular body, wherein the tubular body comprises a proximal portion and an unreinforced distal portion, wherein the delivery catheter is configured to be received within the inner lumen of the catheter body of the neurocatheter for advancing the neurocatheter together with the delivery catheter through at least a portion of the patient's vasculature without aid of a guide sheath.
P Embodiment 3. A system comprising: a neurocatheter comprising a catheter body defining an inner lumen and having a working length between a proximal end and a distal end of the catheter body, wherein the catheter body comprises a proximal portion and a distal portion, wherein the proximal portion is stiffer than the distal portion; and a navigation catheter comprising a tubular body defining an inner lumen and having a working length between a proximal end and a distal end of the tubular body, wherein the tubular body comprises a proximal portion and a distal portion, wherein the proximal portion is stiffer than the distal portion, wherein the navigation catheter is configured to be received within the inner lumen of the catheter body of the neurocatheter for advancing the neurocatheter together with the navigation catheter through at least a portion of the patient's vasculature without aid of a guide sheath.
P Embodiment 4. A method of accessing an intracranial vessel, the method comprising: assembling a navigation catheter with a neurocatheter to form a first assembled system of catheters; advancing the first assembled system of catheters through at least a portion of a patient's vasculature from an arterial access site to a first location without aid of a guide sheath, the first location being distal to a brachiocephalic take-off from the aortic arch; withdrawing the navigation catheter from the neurocatheter; advancing a delivery catheter through the neurocatheter; positioning a distal end region of the delivery catheter distal to a distal end of the neurocatheter to form a second assembled system of catheters; and advancing the second assembled system of catheters to at least a second location distal to the first location.
P Embodiment 5. A navigation catheter comprising a tubular body defining an inner lumen and having a working length between a proximal end and a distal end of the tubular body, wherein the tubular body comprises a proximal portion and a distal portion, wherein the proximal portion is stiffer than the distal portion.
P Embodiment 6. A delivery catheter comprising a tubular body defining an inner lumen and having a working length between a proximal end and a distal end of the tubular body, wherein the tubular body comprises a proximal portion and an unreinforced distal portion.
P Embodiment 7. A system comprising: a neurocatheter comprising a catheter body defining an inner lumen and having a working length between a proximal end and a distal end of the catheter body, wherein the catheter body comprises a proximal portion and a distal portion, wherein the proximal portion is stiffer than the distal portion; a navigation catheter comprising a tubular body defining an inner lumen and having a working length between a proximal end and a distal end of the tubular body, wherein the tubular body comprises a proximal portion and a distal portion, wherein the proximal portion is stiffer than the distal portion; and a delivery catheter comprising a tubular body defining an inner lumen and having a working length between a proximal end and a distal end of the tubular body, wherein the tubular body of the delivery catheter comprises a proximal portion and an unreinforced distal portion, wherein the navigation catheter is configured to be received within the inner lumen of the catheter body of the neurocatheter for advancing the neurocatheter together with the navigation catheter through at least a portion of the patient's vasculature without aid of a guide sheath to a first location, and wherein the delivery catheter is configured to be received within the inner lumen of the catheter body of the neurocatheter in exchange for the navigation catheter for advancing the neurocatheter together with the delivery catheter through at least a portion of the patient's vasculature without aid of a guide sheath to at least a second location that is further distal than the first location.

1. A catheter comprising a catheter body defining an inner lumen having a working length between a proximal end and a distal end of the catheter body, wherein the catheter body comprises a proximal portion and a distal portion, wherein the proximal portion is stiffer than the distal portion by at least 10×, wherein the catheter is configured to be advanced through a patient's vasculature without a guide sheath so the distal portion reaches an intracranial vessel without prolapse of the proximal portion.

2. The catheter of claim 1, wherein the catheter body comprises a multi-layer tube comprising an inner liner layer, an outer jacket layer, and a reinforcement layer positioned between the inner liner layer and the outer jacket layer.

3. The catheter of claim 2, wherein the reinforcement layer varies in configuration between the proximal portion and the distal portion of the catheter body.

4. The catheter of claim 2, wherein the reinforcement layer of the proximal portion comprises a hypotube.

5. The catheter of claim 2, wherein the reinforcement layer of the distal portion comprises a coil reinforcement.

6. The catheter of claim 2, wherein the reinforcement layer of the proximal portion is coupled to the reinforcement layer of the distal portion.

7. The catheter of claim 2, wherein the reinforcement layer of the proximal portion is sufficient to prevent prolapse of the catheter body into the descending aorta during advancement of the distal portion into an intracranial vessel.

8. The catheter of claim 2, wherein the reinforcement layer of the proximal portion allows advancement of the distal portion of the catheter body through an intracranial vessel without the use of a long guide catheter.

9. The catheter of claim 4, wherein the hypotube comprises a proximal end region and a distal end region, wherein at least the distal end region has a plurality of cuts.

10. The catheter of claim 9, wherein the plurality of cuts in the distal end region imparts a greater flexibility in the distal end region compared to the proximal end region.

11. The catheter of claim 9, wherein the plurality of cuts are perpendicular, substantially perpendicular, or angled relative to a longitudinal axis of the hypotube.

12. The catheter of claim 9, wherein the plurality of cuts creates a pattern and the pattern changes periodically continuously over a length of the hypotube to impart a greater flexibility distally compared to proximally.

13.-21. (canceled)

22. The catheter of claim 1, wherein a length of the proximal portion is sufficient to position the distal end region of the hypotube to a location distal to the brachiocephalic take-off and proximal of the carotid siphon of the internal carotid artery when the catheter is advanced from a femoral access site.

23. The catheter of claim 4, wherein a length of the proximal portion comprising the hypotube is sized to avoid advancing substantially into the skull when the catheter is advanced from a femoral access site or a radial access site.

24.-88. (canceled)

89. An intravascular catheter comprising a catheter body having a proximal section, a distal section, and a transition section disposed between the proximal section and the distal section,
wherein the proximal section of the catheter body comprises a proximal end region of a metallic hypotube comprising a first plurality of cuts forming a first plurality of tube segments, the proximal end region having a proximal bending stiffness along a proximal length that is greater than about 10 N,
wherein the transition section of the catheter body comprises a distal end region of the metallic hypotube comprising a second plurality of cuts forming a second plurality of tube segments, the distal end region having a distal bending stiffness and a distal length, wherein the distal bending stiffness changes over the distal length by at least 10×, the distal length being less than about 30 cm, and
wherein the distal section of the catheter body comprises a flexible polymer tube including a coil reinforcement disposed between an inner polymer layer and an outer polymer layer.

90. The catheter of claim 89, wherein the catheter body has a working length of at least 110 cm to about 135 cm and wherein the metallic hypotube has a length of about 90 cm to about 115 cm.

91. The catheter of claim 89, wherein the proximal length is up to about 80 cm.

92. The catheter of claim 89, wherein the distal section has a bending stiffness in a range that allows extension of the distal section of the catheter body through an intracranial vessel.

93. The catheter of claim 89, wherein the proximal section has a bending stiffness range that prevents prolapse of the proximal section of the catheter body into an aorta upon advancement of the distal section into the intracranial vessel.

94. A neurocatheter comprising a catheter body defining an inner lumen having a working length between a proximal end and a distal end of the catheter body, wherein the catheter body comprises a proximal section, a transition section and a distal section, wherein the proximal section is stiffer than the distal section by at least 10×,
wherein the catheter is capable of being advanced through a patient's vasculature to the neurovasculature without being positioned inside an outer catheter except for a short introducer sheath at the access location having a length less than about 30 cm, so the distal section reaches the middle cerebral artery without prolapse of the proximal section.
